(19) United States
(12) Patent Application Publication
(10) Pub. No.: US 2019/0351029 A1
(43) Pub. Date: Nov. 21, 2019

(54) MODULATION OF ISCHEMIC CELL BIOENERGETICS

(71) Applicant: East Carolina University, Greenville, NC (US)

(72) Inventors: Joseph Matthew McClung, Greenville (US); Terence Ryan, Gainesville (US)

(21) Appl. No.: 16/469,608

(22) PCT Filed: Feb. 13, 2018
(86) PCT No.: PCT/US2018/018040

§ 371 Date: Jun. 13, 2019

Related U.S. Application Data
(60) Provisional application No. 62/458,175, filed on Feb. 13, 2017.

(51) Int. Cl. A61K 38/44; A61K 38/45; A61P 9/10; A61K 31/155; A61K 31/4453; A61K 31/40; A61K 31/495; A61K 31/4375; A61K 31/454; A61K 31/4709; C12Q 1/6883

(52) U.S. Cl. CPC A61K 38/44; C12Q 2600/158; A61K 38/45; C12Y 207/01105; A61P 9/10; A61K 31/155; A61K 31/4453; A61K 31/40; A61K 31/495; A61K 31/4375; A61K 31/454; A61K 31/4709; C12Q 1/6883; C12Q 2600/106; C12Y 109/03001

(57) ABSTRACT

Methods of treating ischemia by modulating ischemic cell bioenergetics are described. For example, the methods include the administration of small molecule, polypeptide, and/or genetic agents that modulate oxidative metabolism and/or glycolytic metabolism in ischemic cells, such as ischemic muscle cells. In some embodiments, the agent is adapted to deliver Cox6a2 or PFKFB3 to the cell. Also described are related pharmaceutical compositions and kits for the treatment of ischemia and ischemic injury related to, for instance, such as peripheral arterial disease, stroke, myocardial infarction, and diabetes.

### RELATED APPLICATIONS

The presently disclosed subject matter claims the benefit of U.S. Provisional Patent Application Ser. No. 62/458,175, filed Feb. 13, 2017; the disclosure of which is incorporated herein by reference in its entirety.

### GOVERNMENT INTEREST

This invention was made with government support under HL103797 and HL125695 awarded by NIH. The government has certain rights in the invention.

### TECHNICAL FIELD

The presently disclosed subject matter relates to methods of treating ischemia and diseases and/or conditions involving ischemia, such as peripheral arterial disease (PAD). The methods can involve the administration of polynucleotide-containing, polypeptide-containing, and/or small molecule agents that modulate oxidative and/or glycolytic metabolism in ischemic cells. The presently disclosed subject matter further relates to pharmaceutical formulations and kits comprising such agents.

### ABBREVIATIONS

%=percent
μmol=micromole
AAV=adeno-associated virus
ADP=adenosine diphosphate
ATP=adenosine triphosphate
CLI=critical limb ischemia
cm=centimeter
Cox6a2=cytochrome c oxidase 6a2 subunit
CSA=cross sectional area
d=day
ECAR=extracellular acidification rate
EDL=extensor digitorum longus
eMyHC=embryonic myosin heavy chain
GFP=green fluorescent protein
HLI=hindlimb ischemia
HND=hypoxia and nutrient deprivation
Hz=Hertz
IC=intermittent claudication
LDPI=laser Doppler perfusion imaging
mg=milligram
min=minutes
mM=millimolar
mN=millinewton
MOI=multiplicity of infection
MPC=muscle progenitor cells
mRNA=messenger RNA
nmol=nanomole
OCR=oxygen consumption rate
PAD=peripheral arterial disease
PFKFB3=6-phosphofructo-2-kinase/fructose-2,6-bisphosphatase 3
pmol=picomole
Polg=polymerase gamma
qRT-PCR=quantitative reverse transcriptase polymerase chain reaction
sec=seconds
TA=tibialis anterior
vg=virus genomes

### BACKGROUND

Peripheral artery disease (PAD) is the third leading cause of atherosclerotic cardiovascular mortality (see Fowkes et al., Lancet 2013, 382:1329-40), with an estimated age-adjusted prevalence of at least 12% in the United States. See Ostchega et al., J Am Geriatr Soc 2007, 55:583-9. PAD pathology is caused by atherosclerotic obstruction of the peripheral arteries and manifests itself as either anti-symptomatic, intermittent claudication (IC), or the more severe critical limb ischemia (CLI), which results in chronic rest pain and/or tissue necrosis. CLI carries substantially high morbidity and mortality rates. For example, CLI patients have a risk of major amputation or death that approaches 40% in one year. See Dormandy et al., Semin Vasc Surg 1999, 12:142-7; Hirsch et al., Jama 2001, 286:1317-24; and Taylor et al., Journal of the American College of Surgeons 2009, 208:770-8.

Current clinical interventions for CLI have been aimed at revascularization/neovascularization via gene/growth factor therapy. However, there is an ongoing need for additional methods and/or compositions for treating CLI and PAD, as well as other conditions related to acute or chronic ischemia. In particular, there is an ongoing need for methods and/or compositions for treating ischemia that target different mechanisms underlying the pathological response to ischemia and/or that reduce necrotic tissue loss due to ischemia.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method of treating ischemia in a subject, the method comprising: administering to the subject a composition comprising an agent adapted to modulate oxidative and/or glycolytic metabolism in ischemic cells in the subject; and treating at least one symptom associated with the ischemia in the subject. In some embodiments, treating at least one symptom associated with the ischemia comprises treating at least one symptom associated with ischemic injury, and wherein the symptom associated with ischemic injury comprises tissue necrosis, myopathy, fibrosis or vascular deficiency. In some embodiments, the ischemia and/or ischemic injury is caused by one or more of stroke, myocardial infarction, ischemic limb pathology, peripheral arterial disease, including peripheral arterial disease compromising intermittent claudication, critical limb ischemia, trauma, diabetes mellitus, and acute limb ischemia.

In some embodiments, the agent is adapted to modulate oxidative metabolism in mitochondria in ischemic cells in the subject. In some embodiments, the agent is adapted to provide Cox6a2 to the ischemic cells in the subject. In some embodiments, the administering comprises administering a polynucleotide encoding a Cox6a2 polypeptide to the subject. In some embodiments, the polynucleotide encoding the Cox6a2 polypeptide is operably coupled to a targeting vector capable of causing the expression of the Cox6a2 polypeptide in at least one of a muscle cell, fibroblast, stem cell, pericyte, and endothelial cell.

In some embodiments, the agent is adapted to modulate glycolytic metabolism in ischemic cells in the subject. In some embodiments, the agent is adapted to provide PFKFB3 to the ischemic cells in the subject. In some embodiments, the administering comprises administering a polynucleotide encoding a PFKFB3 polypeptide to the subject. In some embodiments, the polynucleotide encoding the PFKFB3 polypeptide is operably coupled to a targeting vector capable of causing the expression of the PFKFB3 polypeptide in at least one of a muscle cell, fibroblast, stem cell, pericyte, and endothelial cell.

In some embodiments, the agent comprises one or more small molecule pharmacological agents. In some embodiments, the one or more small molecule pharmacological agents are selected from the group comprising metformin, phenformin, biperiden hydrochloride, clemastine, meclizine, berberine chloride, vinpocetine, pimozide and mefloquine.

In some embodiments, the composition comprises a liposome, a nanoparticle, plasmid DNA, recombinant adenovirus, recombinant adeno-associated virus, recombinant lentivirus and combinations thereof. In some embodiments, administering the composition to the subject comprises one or more of intramuscular injection, percutaneous injection, intraperitoneal injection, intravenous injection and oral consumption.

In some embodiments, administering the composition to the subject increases one or more of muscle fiber cross-sectional area, capillary density, muscle function, muscle regeneration, stem cell activity, vascular density, and vascular luminal diameter. In some embodiments, administering the composition to the subject causes an increase in myotube diameter, a change in myotube phenotype, a change in contractile function, an increase in stem cell or satellite cell activity/myogenesis, an increase in mitochondrial number or respiratory function, an increase in autophagic flux, decreased DNA fragmentation or combinations thereof. In some embodiments, administering the composition to the subject causes one or more of increased expression of vascular endothelial growth factor (VEGF), neuropilin (Nrp-1), vascular endothelial growth factor receptor 1 (Flt), vascular endothelial growth factor receptor 2 (Flk), myogenin, myoD, Tmem8c (myomaker) and muscle RING-finger protein 1 (MuRF-1), PGC1-alpha, opa1, Drp1, Mitofusion (Mfn) 1 or 2 and decreased in expression of myostatin.

In some embodiments, the method further comprises, prior to administering to the subject the composition comprising an agent adapted to modulate oxidative and/or glycolytic metabolism in ischemic cells, (i) obtaining a sample from the subject, wherein said sample comprises myofibers from muscle; and (ii) measuring mitochondrial function in the sample from said subject. In some embodiments, measuring mitochondrial function in the sample comprises determining one or more of a Complex IV oxygen consumption rate below 2,000 picomoles per second per milligram myofiber, a Complex II₃ oxygen consumption rate of below 1,000 picomoles per second per milligram myofiber, and/or a Complex I+II₃ oxygen consumption rate of below 1,000 picomoles per second per milligram microfiber.

In some embodiments, the presently disclosed subject matter provides a pharmaceutical composition for treating ischemia in a subject, the composition comprising an agent adapted to modulate oxidative and/or glycolytic metabolism in ischemic cells in the subject; and a pharmaceutically acceptable excipient.

In some embodiments, the presently disclosed subject matter provides a kit comprising a pharmaceutical composition for treating ischemia in a subject, the composition comprising an agent adapted to modulate oxidative and/or glycolytic metabolism in ischemic cells in the subject, and a pharmaceutically acceptable excipient; and a delivery device for administering the pharmaceutical composition to a subject.

In some embodiments, the presently disclosed subject matter provides a method of classifying a subject having peripheral arterial disease (PAD) as unlikely to respond to an endovascular therapeutic intervention, a revascularization therapeutic intervention and/or a therapeutic intervention comprising physical activity, the method comprising: providing a sample from a subject having PAD, wherein the sample comprises myofibers from skeletal muscle; measuring mitochondrial function in the sample; and classifying the subject as being unlikely to respond to an endovascular therapeutic intervention, a revascularization therapeutic intervention and/or a therapeutic intervention comprising physical activity based on mitochondrial function. In some embodiments, the method comprises classifying the subject as being unlikely to respond to an endovascular therapeutic intervention, a revascularization therapeutic intervention and/or a therapeutic intervention comprising physical activity when the sample has a Complex IV oxygen consumption rate below 2,000 picomoles per second per milligram myofiber, a Complex II₃ oxygen consumption rate of below 1,000 picomoles per second per milligram myofiber, and/or a Complex I+II₃ oxygen consumption rate of below 1,000 picomoles per second per milligram microfiber.

Therefore, it is an object of the presently disclosed subject matter to provide a method for treating ischemia, as well as related methods, pharmaceutical compositions and kits.

An object of the presently disclosed subject matter having been stated hereinabove, and which is addressed in whole or in part by the presently disclosed subject matter, other objects will become evident as the description proceeds when taken in connection with the accompanying drawings and examples as best described hereinbelow.

### BRIEF DESCRIPTION OF THE DRAWINGS

FIG. 1A is a graph showing skeletal muscle mitochondrial respiratory function (JO₂; in picomoles per second per milligram dry weight (pmols/sec/mg dry wt)) measured in permeabilized myofiber samples from skeletal muscle biopsy specimens from the gastrocnemius of healthy adults (HA, filled circles), intermittent claudicants (IC, striped circles), and critical limb ischemia (CLI, unfilled circles) patients. Data is shown, from left to right, for oxygen consumption supported by Complex I₄, Complex I₃ (state 3), Complex I+II₃, Complex II₃, cytochrome C (CYT C) and Complex IV. CLI patients displayed decreased Complex I₃, I+II₃, II₃, and IV supported oxygen consumption compared to both IC and HA patients. Error bars are 95% confidence interval (CI). NS=not significant; *P,0.05; **P<0.01; ***P<0.001; ****P<0.0001.

FIG. 1B is a graph of mitochondrial content in the samples from FIG. 1A as assessed by citrate synthase activity (in micromole per minute per gram (μmol/min/g). Data related to healthy adults (HA) is shown in filled circles, data related to intermittent claudicants (IC) is shown in striped circles, and data related to critical limb ischemia patients (CLI) is shown in unfilled circles. Error bars are 95% confidence interval (CI). NS=not significant.

FIG. 1C is a graph of the mitochondrial content in the samples from FIG. 1A as assessed by cardiolipin content (nanomole per milligram protein; nmol/mg protein). Data related to healthy adults (HA) is shown in filled circles, data related to intermittent claudicants (IC) is shown in striped circles, and data related to critical limb ischemia patients (CLI) is shown in unfilled circles. Error bars are 95% confidence interval (CI). NS=not significant; *P,0.05; ****P<0.0001.

FIG. 1D is a series of graphs showing the results of biochemical enzyme assays of muscle lysates of the samples described for FIG. 1A. The graphs are for specific activity assays (measured in milliunits (mU) per unit of citrase synthase (CS) activity) of, from left to right, Complex I, Complex II, Complex III, and Complex IV. Data related to healthy adults (HA) is shown in filled circles, data related to intermittent claudicants (IC) is shown in striped circles, and data related to critical limb ischemia patients (CLI) is shown in unfilled circles. Error bars are 95% confidence interval (CI). NS=not significant; *P,0.05; **P<0.01; ***P<0.001; ****P<0.0001.

FIG. 2A is a graph of the gene ontology (GO) enrichment analysis of gene expression profiles determined by whole genome sequencing of RNA isolated from muscle biopsy samples of the gastrocnemius of healthy adults (HA), intermittent claudicants (IC), and critical limb ischemia (CLI) patients. The

GO enrichment analysis indicates that the most significant gene expression changes were related to mitochondria.

FIG. 2B is a series of graphs showing the messenger RNA (mRNA) changes of selected genes (Cox6a2, Cox6a1, ATP5a1, NDUFA1, MRPL15, and UQCRFS1, from left to right) from the gene expression profiles described for FIG. 2A as validated by quantitative reverse transcriptase polymerase chain reaction (qRT-PCR). Each graph provides data for, from left to right, healthy adults (HA, bars with narrowly spaced stripes running from bottom left to top right), intermittent claudicants (IC, bars with intermediately spaced stripes running from top left to bottom right), and critical limb ischemia (CLI, bars with more widely spaced stripes running from bottom left to upper right) patients. ***P<0.001; ****P<0.0001.

FIG. 3 is a series of graphs showing protein expression (measured in absorbance units (AU)) of various proteins in patient muscle specimens from healthy adults (HA, bars with narrowly spaced stripes running from bottom left to upper right), intermittent claudicants (IC, bars with intermediately spaced stripes running from upper left to bottom right), and critical limb ischemia (CLI, bars with more widely spaced stripes running from bottom left to upper right) patients as determined by quantification of Western blotting using standard densitometry. NS=not significant; *P<0.05, **P<0.01, ****P<0.0001.

FIG. 4A is a schematic drawing showing the differentiation of isolated myoblasts into myotubes by serum withdrawal. The isolated myoblasts were obtained from primary muscle progenitor cells (satellite cells) isolated from muscle biopsies.

FIG. 4B is a graph showing cellular respiration in myotubes from healthy adults (HA, filled circles), intermittent claudicants (IC, striped circles), and critical limb ischemia (CLI, unfilled circles) patients. Oxygen consumption rate (OCR) is measured in picomoles per minute per milligram (pmols/min/mg).

FIG. 4C is a graph showing the quantification of cellular respiration in myotubes from healthy adults (HA, bars with narrowly spaced stripes running from lower left to upper right), intermittent claudicants (IC, bars with intermediately spaced stripes running from upper left to lower right), and critical limb ischemia (CLI, bars with more widely spaced stripes running from lower left to upper right) patients under different substrate/inhibitor combinations, indicating impaired basal, maximal, and Complex IV-linked respiration in cells from CLI patients. N=8 for HA, N=7 for IC, and N=8 for CLI. *P<0.05, **P<0.01, ***P<0.001, ****P<0.0001.

FIG. 4D is a graph showing citrase synthase activity (in micromoles per minute per milligram (μmol/min/mg)) in myotubes from healthy adults (HA, bar on left), intermittent claudicants (IC, middle bar), and critical limb ischemia (CLI, bar on right) patients. NS=not significant.

FIG. 4E is a graph showing the quantification of mitochondrial volume from z-stack confocal imaging of fluorescently labeled mitochondria in muscle progenitor cells (MPC) from healthy adults (HA, bar on left), intermittent claudicants (IC, middle bar), and critical limb ischemia (CLI, bar on right) patients. N=4 for HA; N=4 for IC; and N=4 for CLI. NS=not significant.

FIG. 5A is a graph showing the quantification of laser doppler perfusion imaging (LDPI) paw perfusion in mice from different strains (BL6 (filled circles connected with heavy line), BALB/c (circles with stripes from bottom left to top right and connected by dashed line), the first generation offspring of BL6xBALB/c (F1; circles with stripes from bottom left to top right and connected by solid line), 129s1/SvImJ (circles with stripes from top left to bottom right and connected by solid line), A/J (circles with stripes from top left to bottom right and connected by dashed line), and C3H/HeJ (unfilled circles connected by dashed line) prior to (Pre), on the same day as unilateral hindlimb ischemia (HLI) surgery (d0), and during recovery from surgery (day 7 post-surgery (d7)). *P<0.05.

FIG. 5B is a graph showing the mitochondrial respiratory function (oxygen consumption rate (OCR) as a percentage of control (% Control)) in mitochondria isolated from the plantarflexor muscles of control and ischemic limbs of mice from different strains (BL6 (bars with narrowly spaced stripes from bottom left to top right), BALB/c (bars with narrowly spaced stripes from top left to bottom right), the first generation offspring of BL6xBALB/c (F1, bars with intermediately spaced stripes from top left to bottom right), 129s1/SvImJ (bars with intermediately spaced stripes from bottom left to top right), A/J (bars with widely spaced stripes from bottom left to top right), and C3H/HeJ (bars with widely spaced stripes from top left to bottom right) seven days after unilateral hindlimb ischemia (HLI) surgery. *P<0.05, **P<0.01, ***P<0.001, ****P<0.0001 versus C57BL/6J.

FIG. 5C is a graph showing citrase synthase activity as assessed in isolated mitochondrial preps from control and ischemic limbs of mice from different strains (BL6 (bars with narrowly spaced stripes from bottom left to top right), BALB/c (bars with narrowly spaced stipes from top left to bottom right), the first generation offspring of BL6xBALB/c (F1, bars with intermediately spaced stripes from top left to bottom right), 129s1/SvImJ (bars with intermediately spaced stripes from bottom left to top right), NJ (bars with widely spaced stripes from bottom left to top right), and C3H/HeJ (bars with widely spaced stripes from top left to bottom right) seven days after unilateral hindlimb ischemia (HLI) surgery. NS=not significant.

FIG. 6A is a graph showing the quantification of laser doppler perfusion imaging (LDPI) limb perfusion of BL6 (filled diamonds) and BALB/c (unfilled diamonds) mice prior to (Pre), on the same day as unilateral hindlimb ischemia (HLI) surgery (d0), and during recovery from surgery (up to 56 days post-HLI). *P<0.05.

FIG. 6B is a graph showing assessment of mitochondrial function in mitochondria isolated from the plantarflexor muscles of BL6 (striped bars) and BALB/c (unfilled bars) mice under non-surgical conditions (Cage Control) using high resolution respirometry. Oxygen consumption rate (OCR) is measured in picomoles per second per milligram of mitochondria (pmols/sec/mg mito).

FIG. 6C is a graph showing assessment of mitochondrial function in mitochondria isolated from the plantarflexor muscles of BL6 (striped bars) and BALB/c (unfilled bars) mice at one to 56 days after unilateral hindlimb ischemia (HLI) surgery using high resolution respirometry. Oxygen consumption rate (OCR) was measured in the presence of 10 millimolar (mM) glutamate and 0.5 mM malate and is shown as a percentage of control. **P<0.01, ***P<0.001.

FIG. 6D is a graph showing assessment of mitochondrial function in mitochondria isolated from the plantarflexor muscles of BL6 (striped bars) and BALB/c (unfilled bars) mice at one to 56 days after unilateral hindlimb ischemia (HLI) surgery using high resolution respirometry. Oxygen consumption rate (OCR) was measured in the presence of 10 millimolar (mM) glutamate, 0.5 mM malate, and 4 mM adenosine diphosphate (ADP) to support Complex I respiration and is shown as a percentage of control. ***P<0.001.

FIG. 6E is a graph showing assessment of mitochondrial function in mitochondria isolated from the plantarflexor muscles of BL6 (striped bars) and

BALB/c (unfilled bars) mice at one to 56 days after unilateral hindlimb ischemia (HLI) surgery using high resolution respirometry. Oxygen consumption rate (OCR) was measured in the presence of 10 millimolar (mM) glutamate, 0.5 mM malate, 10 mM succinate and 4 mM adenosine diphosphate (ADP) to support State 3 respiration and is shown as a percentage of control. **P<0.01, ***P<0.001.

FIG. 6F is a graph showing assessment of mitochondrial function in mitochondria isolated from the plantarflexor muscles of BL6 (striped bars) and BALB/c (unfilled bars) mice at one to 56 days after unilateral hindlimb ischemia (HLI) surgery using high resolution respirometry. Complex II supported state 3 respiration is assessed by inhibiting Complex I with 10 millimolar (mM) rotenone. Oxygen consumption rate (OCR) is shown as a percentage of control. **P<0.01, ***P<0.001.

FIG. 6G is a graph showing assessment of mitochondrial function in mitochondria isolated from the plantarflexor muscles of BL6 (striped bars) and BALB/c (unfilled bars) mice at one to 56 days after unilateral hindlimb ischemia (HLI) surgery using high resolution respirometry. Oxygen consumption rate (OCR) was measured in the presence of 10 millimolar (mM) ascorbic acid and 0.4 mM N,N,N′,N′-tetramethyl-p-phenylenediamine (TMPD) to support Complex IV respiration and is shown as a percentage of control. *P<0.05, ***P<0.001.

FIG. 6H is a graph of citrate synthase activity (measured in micromoles per minute per milligram (μmol/min/mg) in isolated mitochondria from the plantarflexor muscles of BL6 (striped bars) and BALB/c (unfilled bars) mice at one to 56 days after unilateral hindlimb ischemia (HLI) surgery.

FIG. 6I is a graph of maximal oxygen consumption rates (OCR) measured in primary muscle, endothelial and fibroblast cells isolated from BL6 (striped bars) and BALB/c (unfilled bars) mice three hours after hypoxia and nutrient deprivation (HND). OCR is expressed as a percentage of the normoxic/normal media control OCR for each cell type. ****P<0.0001.

FIG. 7A is a graph showing the oxygen consumption rates (OCR) in primary muscle cells (differentiated myotubes) from the hindlimb of BL6 and BALB/c mice under normoxia (control; filled circles for BL6 data and striped circles for BALB/c data) and 3 hours of hypoxia and nutrient deprivation (HND, unfilled circles connected by solid line for BL6 data and unfilled circles connected by dashed line for BALB/c data).

FIG. 7B is a graph showing the quantification of maximal oxygen consumption rates, expressed as a percentage of normoxic control rates, in primary muscle cells. BL6 data is on the left and BALB/c data is on the right. ****P<0.0001 versus BL6

FIG. 7C is a graph showing the oxygen consumption rates (OCR) in primary endothelial cells from the hindlimb of BL6 and BALB/c mice under normoxia (control, filled circles for BL6 data and striped circles for BALB/c data) and 3 hours of hypoxia and nutrient deprivation (HND, unfilled circles connected by solid line for BL6 data and unfilled circles connected by dashed line for BALB/c data).

FIG. 7D is a graph showing the quantification of maximal oxygen consumption rates, expressed as a percentage of normoxic control rates, in primary endothelial cells. BL6 data is on the left and BALB/c data is on the right.

FIG. 7E is a graph showing the oxygen consumption rates (OCR) in primary fibroblast cells from the hindlimb of BL6 and BALB/c mice under normoxia (control, filled circles for BL6 data and striped circles for BALB/c data) and 3 hours of hypoxia and nutrient deprivation (HND, unfilled circles connected by solid line for BL6 data and unfilled circles connected by dashed line for BALB/c data).

FIG. 7F is a graph showing the quantification of maximal oxygen consumption rates, expressed as a percentage of normoxic control rates, in primary fibroblast cells. BL6 data is on the left and BALB/c data is on the right.

FIG. 8A is a graph showing the quantification of the percentage of non-muscle area calculated from four times magnified (4×) heamotoxylin and eosin (H&E) stained images of the tibialis anterior muscle of BL6 (striped bars) and BALB/c (unfilled bars) mice 1, 7, 28, or 56 days (d) following unilateral hindlimb ischemia (HLI) surgery. *P<0.05, **P<0.01.

FIG. 8B is a graph showing the ex vivo force production (expressed as a percentage of the contralateral control limb) measured in the extensor digitorum longus muscle of BL6 (filled diamonds) and BALB/c (unfilled diamonds) mice prior to (pre) or up to 56 days (d) after unilateral hindlimb ischemia (HLI) surgery. ****P<0.0001.

FIG. 8C is a graph showing the quantification of blood vessel density in tibialis anterior muscle in BL6 (striped bars) and BALB/c (unfilled bars) mice 1, 7, 28, and 56 days (d) following unilateral hindlimb ischemia (HLI) surgery. Vessel density is quantified from representative immunofluorescence (IF) images stained for blood vessels. **P<0.01.

FIG. 9A is a graph showing representative force tracings of a 100 Hertz (Hz) extensor digitorum longus (EDL) muscle contraction in BL6 mice prior to (Control) or 1, 3, 7, 14, 21, 28, or 56 days (d) following unilateral hindlimb ischemia (HLI) surgery.

FIG. 9B is a graph showing representative force tracings of a 100 Hertz (Hz) extensor digitorum longus (EDL) muscle contraction in BALB/c mice prior to (Control) or 1, 3, 7, 14, 21, 28, or 56 days (d) following unilateral hindlimb ischemia (HLI) surgery.

FIG. 9C is a graph showing the quantified force-frequency contraction curves for BL6 control (filled circles), BALB/c control (striped circles), BL6 ischemic (unfilled circles connected by dashed line) or BALB/c ischemic (unfilled circles connected by dotted line) extensor digitorum longus (EDL) muscles one day after unilateral hindlimb ischemia (HLI) surgery.

FIG. 9D is a graph showing the quantified force-frequency contraction curves for BL6 control (filled circles), BALB/c control (striped circles), BL6 ischemic (unfilled circles connected by dashed line) or BALB/c ischemic (unfilled circles connected by dotted line) extensor digitorum longus (EDL) muscles three days after unilateral hindlimb ischemia (HLI) surgery.

FIG. 9E is a graph showing the quantified force-frequency contraction curves for BL6 control (filled circles), BALB/c control (striped circles), BL6 ischemic (unfilled circles connected by dashed line) or BALB/c ischemic (unfilled circles connected by dotted line) extensor digitorum longus (EDL) muscles seven days after unilateral hindlimb ischemia (HLI) surgery.

FIG. 9F is a graph showing the quantified force-frequency contraction curves for BL6 control (filled circles), BALB/c control (striped circles), BL6 ischemic (unfilled circles connected by dashed line) or BALB/c ischemic (unfilled circles connected by dotted line) extensor digitorum longus (EDL) muscles fourteen days after unilateral hindlimb ischemia (HLI) surgery.

FIG. 9G is a graph showing the quantified force-frequency contraction curves for BL6 control (filled circles), BALB/c control (striped circles), BL6 ischemic (unfilled circles connected by dashed line) or BALB/c ischemic (unfilled circles connected by dotted line) extensor digitorum longus (EDL) muscles twenty-one days after unilateral hindlimb ischemia (HLI) surgery.

FIG. 9H is a graph showing the quantified force-frequency contraction curves for BL6 control (filled circles), BALB/c control (striped circles), BL6 ischemic (unfilled circles connected by dashed line) or BALB/c ischemic (unfilled circles connected by dotted line) extensor digitorum longus (EDL) muscles twenty-eight days after unilateral hindlimb ischemia (HLI) surgery.

FIG. 9I is a graph showing the quantified force-frequency contraction curves for BL6 control (filled circles), BALB/c control (striped circles), BL6 ischemic (unfilled circles connected by dashed line) or BALB/c ischemic (unfilled circles connected by dotted line) extensor digitorum longus (EDL) muscles fifty-six days after unilateral hindlimb ischemia (HLI) surgery.

FIG. 10A is a graph showing the quantification of Cox6a2, ATP5a, and UQCRC2 protein abundance relative to HDP60 (a mitochondrial loading control) in isolated mitochondria from control or ischemic inbred mouse strains (BL6, BALB/c, first-generation BL6xBALB/c, 129s1/SvImJ, NJ, and C3H/HeJ). Ischemic mitochondria were isolated on day 7 following unilateral hindlimb ischemia (HLI) surgery. *P<0.05, **P<0.01, ***P<0.001, ****P<0.0001 versus C57BL/6J.

FIG. 10B is a graph showing the quantification of Cox6a2 protein expression by Western blotting in BL6 (filled circles) and BALB/c (striped circles) mice prior to (Con) or 1, 3, 7, 14, 21, 28, or 56 days following unilateral hindlimb ischemia (HLI) surgery. **P<0.001 versus C57BL/6J.

FIG. 10C is a graph showing the quantification of ATP5a and UQCRC2 protein expression in BL6 and BALB/c mice prior to (Con) or 1, 3, 7, 14, 21, 28, or 56 days following unilateral hindlimb ischemia (HLI) surgery. Data for BL6 ATP5a is shown in filled circles, data for BALB/c ATP5a is shown in striped circles, data for BL6 UQCRC2 is shown in unfilled circles connected by a solid line, and data for BALB/c UQCRC2 is shown in unfilled circles connected by a dashed line. **P<0.001 versus C57BL/6J. NS=not significant.

FIG. 11 is a graph showing the quantification of Cox6a2 protein expression in limb muscle mitochondria from BALB/c mice that had received an intramuscular injection of a control virus encoding green fluorescent protein (GFP, striped circles) or a virus containing a nucleotide encoding Cox6a2 (Cox6a2, unfilled circles). Mitochondria were isolated on the seventh day following unilateral hindlimb ischemica (HLI) surgery. For comparison, data from mice that did not receive an injection or HLI is also shown (Control, filled circles).

FIG. 12A is a graph showing the limb necrosis score of mice at day 7 or day 28 post unilateral hindlimb ischemia (HLI) surgery. Data is shown for BL6 mice (N=30; filled circles), as well as for BALB/c mice that had been injected with an adeno-associated virus (AAV) encoding either green fluorescent protein (GFP; N=64; striped circles) or Cox6a2 (N=64, unfilled circles) prior to surgery. *P<0.05 versus BALB/c-GFP with Mann-Whitney U Test.

FIG. 12B is a graph showing the quantification of limb perfusion measured using laser doppler perfusion imaging (LDPI) in BL6 mice (filled circles) and in BALB/c mice that were injected with an adeno-associated virus (AAV) encoding either green fluorescent protein (GFP; N=64, striped circles) or Cox6a2 (N=64, unfilled circles) prior to unilateral hindlimb ischemia (HLI) surgery. Data is shown for the limb perfusion measured prior to HLI and one (Post), three (d3), seven (d7), fourteen (d14), twenty-one (d21), or twenty-eight (d28) days after HLI.

FIG. 12C is a series of graphs showing mitochondrial function assessed one day (left), seven days (middle) or 28 days (right) post unilateral hindlimb ischemia (HLI) using high-resolution respirometry in mitochondrial isolated from the plantarflexor muscles of the BL6 mice (bars with stripes running from bottom left to top right) or BALB/c mice injected with an adeno-associated virus (AAV) encoding either green fluorescent protein (BALB/c-GFP, bars with stripes running from top left to bottom right) or Cox6a2 (BALB/c-Cox6a2, unfilled bars) prior to HLI. *P<0.05, **P<0.01 versus BALB/c-GFP.

FIG. 12D is a graph showing citrate synthase activity measured spectrophotometrically in BL6 mice (bars with stripes running from bottom left to top right) and in BALB/c mice injected with an adeno-associated virus (AAV) encoding either green fluorescent protein (BALB/c-GFP, bars with stripes running from top left to bottom right) or Cox6a2 (BALB/c-Cox6a2, unfilled bars) prior to unilateral hindlimb ischemia (HLI) surgery. Data was measured prior to HLI (Control) and 1, 7, and 28 days (d) post HLI.

FIG. 12E is a graph showing the quantification of mitochondrial supercomplex formation in control or ischemic limbs of mice injected with an adeno-associated virus (AAV) encoding either green fluorescent protein (GFP) or Cox6a2 (BALB/c-Cox6a2). Mitochondria were isolated at day seven post unilateral hindlimb ischemia (HLI). Data from AAV-GFP control limbs is shown in bars with strips running from bottom left to top right; data from AAV-GFP ischemic limbs is shown in unfilled bars with heavy outline; data from AAV-Cox6a2 control limbs is shown in bars with stripes running from top left to bottom right; and data from AAV-Cox6a2 ischemic limbs is shown in unfilled bars with light outline. *P<0.05 versus within treatment control (HLI effect). ^{φ}P<0.05 versus GFP (virus effect).

FIG. 12F is a pair of graphs showing the specific activity assay of cytochrome c oxidase (Complex IV) in the mitochondrial electron transport system (ETS) performed in isolated mitochondria from control (left) and ischemic (right) limbs at day seven post unilateral hindlimb ischemia (HLI) in BL6 mice or mice treated with an adeno-associated virus (AAV) encoding either green fluorescent protein (BALB/c-GFP) or Cox6a2 (BALB/c-Cox6a2) prior to HLI. In the graph on the left, data from BL6 mice is shown with the heavy line, and data from BALB/c mice with the lighter line. In the graph on the right, data from the BL6 mice is shown in the dashed line; data from BALB/c-GFP mice with the dotted line, and data from BALB/c-Cox6a2 with the heavy solid line.

FIG. 12G is a graph showing the quantification of Complex IV specific activity data from FIG. 12F (normalized to citrate synthase) expressed as a percentage of non-ischemic control rate. ****P<0.0001 versus BL6.

FIG. 13A is a graph showing the quantification of non-myofiber area in hematoxylin and eosin (H&E) stained tibialis anterior muscle following unilateral hindlimb ischemia (HLI) surgery of BL6 mice (bars with stripes running from top left to bottom right) or BALB/c mice injected intramuscularly with adeno-associated virus (AAV) encoding either green fluorescent protein (BALB/c-GFP, bars with stripes running from bottom left to top right) or Cox6a2 (BALB/c-Cox6a2, unfilled bars) prior to HLI. **P<0.01, ***P<0.001, ****P<0.0001 versus BL6. NS=not significant.

FIG. 13B is a graph showing the quantification of the average myofiber cross sectional areas (CSA) in hematoxylin and eosin (H&E) stained tibialis anterior (TA) muscle following unilateral hindlimb ischemia (HLI) surgery of BL6 mice (bars with stripes running from top left to bottom right) or BALB/c mice injected intramuscularly with adeno-associated virus (AAV) encoding either green fluorescent protein (BALB/c-GFP, bars with stripes running from bottom left to top right) or Cox6a2 (BALB/c-Cox6a2, unfilled bars) prior to HLI. *P<0.05, **P<0.01, ***P<0.001, ****P<0.0001 versus BL6.

FIG. 13C is a graph showing the quantification of the number of embryonic myosin heavy chain (eMyHC) positive myofibers at day seven post unilateral hindlimb ischemia (HLI) surgery in tibialis anterior (TA) muscle stained for eMyHC from BL6 mice (bar on left) or BALB/c mice injected intramuscularly with adeno-associated virus (AAV) encoding either green fluorescent protein (BALB/c-GFP, middle bar) or Cox6a2 (BALB/c-Cox6a2, bar on right) prior to HLI. **P<0.01, ***P<0.001 versus BL6.

FIG. 14A is a graph showing the oxygen consumption under normal growth conditions (Control) or hypoxia and nutrient deprivation (HND) in undifferentiated primary muscle cells (myoblasts) isolated from BL6 (filled circles for control and unfilled circles connected with solid line for HND) and BALB/c mice (striped circles for control and unfilled circles connected by dashed line for HND).

FIG. 14B is a graph showing the affect of adenoviral manipulation on myoblast cell proliferation in BL6 mice. Mice were injected with an adenovirus encoding a scrambled construct (Ad-Scram, filled squares) or a nucleotide designed for knockdown of Cox6a2 in BL6 cells (Ad-shCox6a2, unfilled squares). Data is also shown for mice that were not injected with an adenovirus (Control, filled circles).

FIG. 14C is a graph showing the affect of adenoviral manipulation on myoblast cell proliferation in BALB/c mice. Mice were injected with an adenovirus encoding a scrambled construct (Ad-Scram, filled squares) or a nucleotide designed for overexpression of Cox6a2 in BALB/c cells (Ad-Cox6a2^{OE,} unfilled squares). Data is also shown for mice that were not injected with an adenovirus (Control, filled circles).

FIG. 15A is a schematic drawing showing the isolation of primary muscle cells from the hindlimb of BL6 and BALB/c mice and Cox6a2 expression manipulation via adenovirus treatment during myoblast differentiation followed by either normoxia (control) or 3 hours of hypoxia and nutrient deprivation (3HND) to mimic peripheral arterial disease (PAD) in vitro.

FIG. 15B is a graph showing the quantified Cox6a2 messenger RNA levels measured via quantitative reverse transcriptase polymerase chain reaction (qRT-PCR) in primary muscle cells isolated from the hindlimb of BL6 (bars with stripes running from bottom left to top right) or BALB/c (bars with stripes running from top left to bottom right) mice that were treated with: no adenovirus (Con), an adenovirus encoding a scrambled construct (Scram), an adenovirus encoding a nucleotide designed for knockdown of Cox6a2 in BL6 mice (Cox6a2^{shRNA}), or an adenovirus encoding a nucleotide designed for overexpression of Cox6a2 in BALB/c cells (Cox6a2^{OE}). Data is shown for cells under normoxic conditions (−) and after three hours of hypoxia and nutrient deprivation (3HND). *P<0.05, for within group effect (BL6 versus BALB/c); ^{φ}P<0.05 (Scram versus Cox6a2^{shRNA} or Cox6a2^{OE}).

FIG. 15C is a graph showing cellular respiration in BL6 myotubes infected with adenovirus encoding scrambled (SCRAM) or Cox6a2^{shRNA} constructs after normoxia (control, filled circles for SCRAM and striped circles for Cox6a2^{shRNA}) or three hours of hypoxia and nutrient deprivation (3HND, unfilled circles connected by solid line for SCRAM and unfilled circles connected by dashed line for Cox6a2^{shRNA}) treatment.

FIG. 15D is a graph showing cellular respiration in BALB/c myotubes infected with adenovirus encoding scrambled (SCRAM) or Cox6a2^{OE} constructs after normoxia (control; filled circles for SCRAM and striped circles for Cox6a2^{OE}) or three hours of hypoxia and nutrient deprivation (3HND, unfilled circles connected by solid line for SCRAM and unfilled circles connected by dashed line for Cox6a2^{OE}) treatment.

FIG. 15E is a graph showing the quantification of the maximal respiratory capacity from FIGS. 15C and 15D.

FIG. 15F is a graph showing the quantification of myoblast fusion index for primary muscle cells treated as described in FIG. 15B.

FIG. 15G is a graph showing the quantification of myosin heavy chain (MyHC) positive area in primary muscle cells treated as described in FIG. 15B.

FIG. 16A is a pair of graphs showing (left) Cox6a2 and (right) Cox6a1 messenger RNA expression in muscle biopsy specimens from age-matched non-peripheral arterial disease (PAD) control subjects (non-PAD, bars with stripes running from bottom left to top right) and patients with clinically diagnosed critical limb ischemia (CLI, bars with stripes running from top left to bottom right). The mRNA expression was determined via quantitative reverse transcriptase polymerase chain reaction (qRT-PCR) and corrected for 18 s and normalized to non-PAD.

FIG. 16B is a series of graphs showing the quantification of (left) Cox6a2, (middle) HSP60, and (right) GAPDH expression determined by

Western blotting in muscle biopsy specimens from age-matched non-peripheral arterial disease (PAD) control subjects (non-PAD, bars with stripes running from bottom left to top right) and patients with clinically diagnosed critical limb ischemia (CLI, bars with stripes running from top left to bottom right).

FIG. 16C is a schematic diagram showing the isolation of primary human muscle cells from muscle biopsy specimens obtained from non-peripheral arterial disease (PAD) control subjects (non-PAD) and patients with clinically diagnosed critical limb ischemia (CLI). Cox6a2 expression is manipulated via adenovirus treatment during myoblast differentiation.

FIG. 16D is a graph showing the quantification of Cox6a2 messenger RNA in human myoblasts treated as described in FIG. 16C. Data is included for no virus controls (con) for myoblasts from non-peripheral arterial disease (non-PAD, bars with stripes running from bottom left to top right) subjects and critical limb ischemia (CLI, bars with stripes running from top left to bottom right) patients, as well as for myoblasts from non-PAD and CLI subjects treated with an adenovirus encoding a scrambled nucleotide construct (Scram), an adenovirus encoding a nucleotide designed for the knockdown of Cox6a2 (Cox6a2^{shRNA}) or the overexpression of Cox6a2 (Cox6a2^{OE}). *P<0.05 versus Non-PAD. ^{φ}P<0.001 for virus effect (Scram vs. Cox6a2^{shRNA} or Cox6a2^{OE}).

FIG. 16E is a graph showing cellular respiration in myotubes from non-peripheral arterial disease (non-PAD) subjects infected with adenovirus encoding scrambled (Scram, filled circles) or Cox6a2 knockdown (Cox6a2^{shRNA}, unfilled circles) constructs.

FIG. 16F is a graph showing cellular respiration measured in myotubes from critical limb ischemia (CLI) patients infected with adenovirus encoding scrambled (Scram, filled circles) or Cox6a2 overexpression (Cox6a2^{OE}, unfilled circles) constructs.

FIG. 16G is a graph showing the quantification of the maximal respiratory capacity from the data shown in FIGS. 16E and 16F.

FIG. 16H is a graph showing the quantification of myoblast fusion index (shown as a percentage (%)) determined from immunofluorescent images of primary muscle cells from non-peripheral arterial disease (non-PAD, bars with stripes running from bottom left to top right) and critical limb ischemia (CLI, bars with stripes running from top left to bottom right) subjects that were infected with adenovirus encoding scrambled (Scram), Cox6a2 knockdown (Cox6a2^{shRNA}), or Cox6a2 overexpression (Cox6a2^{OE}) constructs and then differentiated into myotubes. *P<0.05 versus Non-PAD. ^{φ}P<0.001 for virus effect (Scram vs. Cox6a2^{shRNA} or Cox6a2^{OE}).

FIG. 16I is a graph showing the quantification of myosin heavy chain (MyHC) positive area from immunofluorescent images of primary muscle cells from non-peripheral arterial disease (non-PAD, bars with stripes running from bottom left to top right) and critical limb ischemia (CLI, bars with stripes running from top left to bottom right) subjects that were infected with adenovirus encoding scrambled (Scram), Cox6a2 knockdown (Cox6a2^{shRNA}), or Cox6a2 overexpression (Cox6a2^{OE}) constructs and then differentiated into myotubes. *P<0.05 versus Non-PAD. ^{φ}P<0.001 for virus effect (Scram vs. Cox6a2^{shRNA} or Cox6a2^{OE}).

FIG. 17A is a graph showing the cell fusion index (shown as a percentage (%)) of human primary muscle cells from critical limb ischemia (CLI) patients infected with an adenovirus encoding a nucleotide for human Cox6a2 overexpression (hCox6a2^{OE}) or for mouse Cox6a2 overexpression (mCox6a2^{OE}). Data is also shown for non-infected CLI cells (Control). *P≤0.05.

FIG. 17B is a graph showing the area percentage (%) of myosin heavy chain in human primary muscle cells from critical limb ischemia (CLI) patients infected with an adenovirus encoding a nucleotide for human Cox6a2 overexpression (hCox6a2^{OE}) or mouse Cox6a2 overexpression (mCox6a2^{OE}). Data is also shown for non-infected CLI cells (Control). *P<0.02.

FIG. 18A is a graph showing paw perfusion in mice that are heterozygous (Polg^{+/-}, unfilled squares) or homozygous (Polg^{+/+}, unfilled triangles) for a mutant allele of mitochonridal DNA (mtDNA) polymerase gamma (Polg) prior to (Pre) and up to seven days (d7) following unilateral hindlimb ischemia (HLI). Data is also shown for wild-type mice (Polg^{-/-}, filled circles). Paw perfusion in the ischemic limb is shown as a percentage (%) of perfusion in the control (non-ischemic) limb.

FIG. 18B is a graph showing ischemic lesion area (expressed as a percentage (%) of the total area of the tibialis anterior (TA) muscle) after unilateral hindlimb ischemia (HLI) surgery in mice that are heterozygous (Polg^{+/-}, unfilled squares) or homozygous (Polg^{+/+}, unfilled triangles) for a mutant allele of mitochonridal DNA (mtDNA) polymerase gamma (Polg). Data is also shown for wild-type mice (Polg^{-/-}, filled circles). ****P<0.0001. NS=not significant.

FIG. 18C is a graph showing peak specific force in the control or ischemic skeletal muscle of mice that are heterozygous (Polg^{+/-}, bars with stripes running from top left to bottom right) or homozygous (Polg^{+/+}, unfilled bars) for a mutant allele of mitochonridal DNA (mtDNA) polymerase gamma (Polg) following unilateral hindlimb ischemia (HLI) surgery. Data is also shown for wild-type mice (Polg^{-/-}, bars with stripes running from bottom left to top right). ***P<0.001.

FIG. 19A is a graph of the enriched gene ontology (GO) terms obtained from a study of differentially expressed genes in RNA isolated from ischemic muscle biopsy samples (three days post unilateral hindlimb ischemia (HLI) surgery) from mice homozygous for a mutant allele of mitochonridal DNA (mtDNA) polymerase gamma (Polg) compared to wild-type control mice. The GO enrichment analysis suggests a reprogramming of glycolytic metabolism.

FIG. 19B is a graph showing the levels of resting blood lactate (millimolar (mM)) in muscle biopsy samples from mice heterozygous (Polg^{+/-}, middle bar) or homozygous (Polg^{+/+}, bar on right) for a mutant allele of mitochonridal DNA (mtDNA) polymerase gamma (Polg). Data is also shown for a wild-type (Polg^{-/-}, bar on left) mice. ****P<0.0001 versus Polg^{-/-}.

FIG. 19C is a graph showing enhanced glycolytic flux (measured as extracellular acidification rate (ECAR)) in isolated primary skeletal muscle cells from mice heterozygous (Polg+/−, striped circles) and homozygous (Polg^{+/+}, unfilled circles connected by dashed line) for a mutant allele of mitochonridal DNA (mtDNA) polymerase gamma (Polg). Data for wild-type mice (Polg^{-/-}, filled circles) is also shown.

FIG. 19D is a graph showing the relative expression of messenger RNA (mRNA) for 6-phosphofructo-2-kinase/fructose-2,6-bisphosphatase 3 (PFKFB3) in the non-ischemic (Control) or ischemic limbs of mice that are homozygous for polymerase gamma (Polg^{+/+}, unfilled bars) and wild-type mice (Polg^{-/-}, striped bars) following unilateral hindlimb ischemia (HLI). *P<0.05 versus Polg^{-/-}. ^{φ}P<0.05 versus non-ischemic control.

FIG. 19E is a graph showing the relative expression of messenger RNA (mRNA) for phosphofructokinase (PFKM) in the non-ischemic (Control) or ischemic limbs of mice that are homozygous for polymerase gamma (Polg^{+/+}, unfilled bars) and wild-type mice (Polg^{-/-}, striped bars) following unilateral hindlimb ischemia (HLI). *P<0.05 versus Polg^{-/-}. ^{φ}P<0.05 versus non-ischemic control.

FIG. 19F is a graph showing the ratio of 6-phosphofructo-2-kinase/fructose-2,6-bisphosphatase 3 (PFKFB3) protein expression to total protein expression in ischemic muscle from mice that are homozygous (Polg^{+/+}, bar on right) or heterozygous (Polg^{+/-}, middle bar) for polymerase gamma (Polg) and for wild-type mice (Polg^{-/-}, bar on left) following unilateral hindlimb ischemia (HLI).

FIG. 20A is a graph showing relative messenger RNA (mRNA) expression levels of 6-phosphofructo-2-kinase/fructose-2,6-biphosphatase (PFKFB3) in skeletal muscle cells (myotubes) treated with an adeno-associated virus containing a control green fluorescent protein (GFP) construct (AAV-GFP) or containing a construct for the overexpression of PFKFB3 (AAV-PFKFB3). Data is also shown for non-virus treated control (Control). ****P<0.0001 versus control.

FIG. 20B is a graph showing basal and maximal glycolytic flux in skeletal muscle cells treated with an adeno-associated virus containing a control green fluorescent protein (GFP) construct (AAV-GFP) or containing a construct for the overexpression of 6-phosphofructo-2-kinase/fructose-2,6-bisphosphatase 3 (AAV-PFKFB3). Data is also shown for non-virus treated control (Control). ***P<0.001 versus control.

FIG. 20C is a graph showing cell survival (presented as a % of cell count for control cells under normoxia conditions) in skeletal muscle cells under normoxia (left) and hypoxia (right) conditions. Cells were treated with an adeno-associated virus containing a control green fluorescent protein (GFP) construct (AAV-GFP) or containing a construct for the overexpression of 6-phosphofructo-2-kinase/fructose-2,6-bisphosphatase 3 (AAV-PFKFB3). Data is also shown for non-virus treated control (Control). ***P<0.001 versus control.

FIG. 20D is a graph showing basal glycolytic flux in human umbilical vein endothelial cells (HUVECs) treated with an adeno-associated virus containing a control green fluorescent protein (GFP) construct (AAV-GFP) or containing a construct for the overexpression of 6-phosphofructo-2-kinase/fructose-2,6-bisphosphatase 3 (AAV-PFKFB3) and further treated with dimethyl sulfoxide (DMSO) or a small molecule inhibitor of PFKFB3, i.e., PKF15. **P<0.01 versus control (AAV-GFP or DMSO); ^{φ}P<0.05 for virus effect.

FIG. 20E is a graph showing maximal glycolytic flux in human umbilical vein endothelial cells (HUVECs) treated with an adeno-associated virus containing a control green fluorescent protein (GFP) construct (AAV-GFP) or containing a construct for the overexpression of 6-phosphofructo-2-kinase/fructose-2,6-bisphosphatase 3 (AAV-PFKFB3) and further treated with dimethyl sulfoxide (DMSO) or a small molecule inhibitor of PFKFB3, i.e., PKF15. **P<0.01 versus control (AAV-GFP or DMSO); ^{φ}P<0.05 for virus effect.

FIG. 20F is a graph showing the level of angiogenesis (as determined by number (#) of endothelial cell tubes formed) in human umbilical vein endothelial cells (HUVECs) treated with an adeno-associated virus containing a control green fluorescent protein (GFP) construct (AAV-GFP) or containing a construct for the overexpression of 6-phosphofructo-2-kinase/fructose-2,6-bisphosphatase 3 (AAV-PFKFB3) and further treated with dimethyl sulfoxide (DMSO) or a small molecule inhibitor of PFKFB3, i.e., PKF15. ***P<0.001, ****P<0.0001 versus control (AAV-GFP or DMSO).

FIG. 21A is a graph showing relative messenger RNA (mRNA) expression levels of 6-phosphofructo-2-kinase/fructose-2,6-biphosphatase (PFKFB3) in skeletal muscle cells of BALBc/J mice treated with intramuscular injections of an adeno-associated virus containing a control green fluorescent protein (GFP) construct (AAV-GFP) or containing a construct for the overexpression of PFKFB3 (AAV-PFKFB3) to the hindlimb musculature at 1×10¹¹ vg/muscle three weeks prior to unilateral hindlimb ischemia (HLI) surgery.

FIG. 21B is a graph showing paw perfusion recovery (measured using laser doppler perfusion imaging (LDPI) quantified as a percentage (%) of control limb perfusion) in the mice described for FIG. 21A. Data for mice treated with a virus containing green fluorescent protein (AAV-GFP) is shown in the filled circles, while data from mice treated with the virus containing the construct for overexpression of PFKFB3 (AAV-PFKFB3) is shown in unfilled circles **P<0.01.

FIG. 21C is a graph showing limb necrosis in the mice described for FIG. 21A. Limb necrosis is presented as the proportion of mice per necrosis score (0, 1, 2, 3, or 4, going from a lower to a higher necrosis score). P=0.08.

FIG. 21D is a graph showing muscle force production (measured as peak specific force) in non-ischemic (Control) or ischemic muscle in the mice described for FIG. 21A. Data for mice treated with a virus containing green fluorescent protein (AAV-GFP) is shown in bars with stripes running from bottom left to top right, while data from mice treated with the virus containing the construct for overexpression of PFKFB3 (AAV-PFKFB3) is shown in bars with stripes running from top left to bottom right. P=0.11

FIG. 22 is a series of graphs showing (left) the level of 6-phosphofructo-2-kinase/fructose-2,6-biphosphatase (PFKFB3) expression, (middle) the level of GAPDH expression, and (right) total protein expression determined via Western blotting in human critical limb ischemia (CLI, bars with stripes running from top left to bottom right) patient muscle biopsy samples. For comparison, data is also shown for healthy adults without peripheral arterial disease (non-PAD, bars with stripes running from bottom left to top right). ***<0.001. NS=not significant.

FIG. 23A is graph showing the effects of treating cardiomyoblast cells with various small molecules prior to 72 hour experimental hypoxia. Cell count was determined following nuclear staining. Cell count is also provided for cells treated with dimethyl sulfoxide (DMSO) under normoxia or experimental hypoxia as a control or comparison.

FIG. 23B is graph showing basal glycolytic flux for the cells described for FIG. 23A.

FIG. 23C is a graph showing maximal glycolytic flux for the cells described for FIG. 23A.

FIG. 24A is graph showing the effects of treating kidney fibroblast cells with various small molecules prior to 72 hour experimental hypoxia. Cell count was determined following nuclear staining. Cell count is also provided for cells treated with dimethyl sulfoxide (DMSO) under normoxia or experimental hypoxia as a control or comparison.

FIG. 24B is graph showing basal glycolytic flux for the cells described for FIG. 24A.

FIG. 24C is a graph showing maximal glycolytic flux for the cells described for FIG. 24A.

FIG. 25A is graph showing the effects of treating primary mouse cortical neurons with various small molecules prior to 72 hour experimental hypoxia. Cell count was determined following nuclear staining. Cell count is also provided for cells treated with dimethyl sulfoxide (DMSO) under normoxia or experimental hypoxia as a control or comparison.

FIG. 25B is graph showing basal glycolytic flux for the neurons described for FIG. 25A.

FIG. 25C is a graph showing maximal glycolytic flux for the neurons described for FIG. 25A.

FIG. 26A is a graph showing the correlation between intermittent claudicant ankle brachial index (ABI) values and the mitochondrial respiration measured for Complex I₄ in permeabilized myofibers from skeletal muscle samples. Mitochondrial respiration is measured as an oxygen consumption rate (OCR) in picomoles per second per milligram (pmol/sec/mg) of myofiber. The Spearman correlation is 0.3784. P=0.4008.

FIG. 26B is a graph showing the correlation between intermittent claudicant ankle brachial index (ABI) values and the mitochondrial respiration measured for Complex I₃ in permeabilized myofibers from skeletal muscle samples. Mitochondrial respiration is measured as an oxygen consumption rate (OCR) in picomoles per second per milligram (pmol/sec/mg) of myofiber. The Spearman correlation is 0.0900. P=0.8595.

FIG. 26C is a graph showing the correlation between intermittent claudicant ankle brachial index (ABI) values and the mitochondrial respiration measured for Complex I+II₃ in permeabilized myofibers from skeletal muscle samples. Mitochondrial respiration is measured as an oxygen consumption rate (OCR) in picomoles per second per milligram (pmol/sec/mg) of myofiber. The Spearman correlation is 0.8469. P=0.0246.

FIG. 26D is a graph showing the correlation between intermittent claudicant ankle brachial index (ABI) values and the mitochondrial respiration measured for Complex II₃ in permeabilized myofibers from skeletal muscle samples. Mitochondrial respiration is measured as an oxygen consumption rate (OCR) in picomoles per second per milligram (pmol/sec/mg) of myofiber. The Spearman correlation is 0.8108. P=0.0349.

FIG. 26E is a graph showing the correlation between intermittent claudicant ankle brachial index (ABI) values and the mitochondrial respiration measured for Complex IV in permeabilized myofibers from skeletal muscle samples. Mitochondrial respiration is measured as an oxygen consumption rate (OCR) in picomoles per second per milligram (pmol/sec/mg) of myofiber. The Spearman correlation is 0.9009. P=0.0095.

FIG. 26F is a graph showing the correlation between Complex IV (C IV) activity (measured in milliunits per unit citrase synthase (mU/U CS) and Cox4 protein abundance in permeabilized myofibers from skeletal muscle samples. The Spearman correlation is 0.7857. P=0.0279.

FIG. 26G is a graph showing the correlation between Complex IV (C IV) activity (measured in milliunits per unit citrase synthase (mU/U CS) and Cox6a2 protein abundance in permeabilized myofibers from skeletal muscle samples. The Spearman correlation is 0.9048. P=0.0046.

### BRIEF DESCRIPTION OF THE SEQUENCE LISTING

SEQ ID NO: 1 is a polynucleotide sequence encoding murine cytochrome c oxidase 6a2 (Cox6a2) subunit.

SEQ ID NO: 2 is a Cox6a2 polypeptide sequence encoded by SEQ ID NO: 1.

SEQ ID NO: 3 is a polynucleotide sequence encoding human Cox6a2.

SEQ ID NO: 4 is a Cox6a2 polypeptide sequence encoded by SEQ ID NO: 3.

SEQ ID NO: 5 is a polynucleotide sequence encoding murine 6-phosphofructo-2-kinase/fructose-2,6-bisphosphatase 3 (PFKFB3).

SEQ ID NO: 6 is a PFKFB3 polypeptide sequence encoded by SEQ ID NO: 5.

SEQ ID NO: 7 is a polynucleotide sequence encoding human 6-phosphofructo-2-kinase/fructose-2,6-bisphosphatase 3 (PFKFB3).

SEQ ID NO: 8 is a PFKFB3 polypeptide sequence encoded by SEQ ID NO: 7.

### DETAILED DESCRIPTION

Critical limb ischemia (CLI) is a severe form of peripheral arterial disease (PAD) and has a high rate of morbidity and mortality outcomes. Currently there are few effective treatments for CLI. To date, methods of treating PAD typically involve revascularization/neovascularization using gene and/or growth factor therapy. However, these methods have largely been ineffective at alleviating negative outcomes, such as necrotic tissue loss. Thus, in accordance with the presently disclosed subject matter, treatments for CLI and other diseases related to or associated with acute or chronic ischemia are provided that target alternative mechanisms behind the pathological response to ischemia. More particularly, in some embodiments, the presently disclosed subject matter relates to methods of modulating ischemic cell bioenergetics, such as by modulating oxidative and/or glycolytic metabolism in ischemic cells. In some embodiments, the presently disclosed subject matter relates to methods of administering an agent adapted to provide Cox6a2 or PFKFB3 to a subject in need of treatment for ischemia.

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a cell” or “an agent” includes a plurality of such cells or agents, and so forth.

Unless otherwise indicated, all numbers expressing quantities of ingredients, reaction conditions, sequence identity, dosages, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

Thus, as used herein, the term “about,” when referring to a value or to an amount of a virus (e.g., titer), dose (e.g. an amount of a gene therapy construct), sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including”, “containing”, or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “cell” refers not only to the particular subject cell (e.g., a living biological cell), but also to the progeny or potential progeny of such a cell. Because certain modifications can occur in succeeding generations due to either mutation or environmental influences, such progeny might not, in fact, be identical to the parent cell, but are still included within the scope of the term as used herein.

The terms “nucleic acid molecule” or “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms “nucleic acid molecule” or “nucleic acid” can also be used in place of “gene”, “cDNA”, or “mRNA”. Nucleic acids can be synthesized, or can be derived from any biological source, including any organism.

The terms “heterologous nucleic acid” or “non-native nucleic acid” refer to a nucleotide sequence that originates from a source foreign to an intended host cell or, if from the same source, is modified from its original form. Thus, a heterologous nucleic acid in a host cell includes a gene that is endogenous to the particular host cell, but which has been modified, for example by mutagenesis or by isolation from native cis-regulatory sequences. The term “heterologous nucleic acid” also includes non-naturally occurring multiple copies of a native nucleotide sequence. The term “heterologous nucleic acid” also encompasses a nucleic acid that is incorporated into a host cell's nucleic acids, however at a position wherein such nucleic acids are not ordinarily found.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, which enable its replication in a host cell. The term “recombinant” is also used to describe a vector (e.g., an adenovirus or an adeno-associated virus) comprising recombinant nucleic acids.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleotide sequences that encode the disclosed amino acid sequences, including but not limited to those disclosed in the corresponding GENBANK® entries. In some embodiments, the gene encodes a polypeptide comprising a sequence of SEQ ID NO:2, SEQ ID NO:4, SEQ ID NO:6, SEQ ID NO:8 or a sequence substantially identical thereto. In some embodiments, the gene comprises a sequence of SEQ ID NO:1, SEQ ID NO:3, SEQ ID NO:5, SEQ ID NO:7 or a sequence substantially identical thereto.

The term “transgene” as used herein refers to a nucleic acid sequence, heterologous to the vector sequences flanking the transgene, which encodes a polypeptide, protein, or other product of interest (e.g., a “therapeutic peptide”). The nucleic acid coding sequence is operatively linked to regulatory components in a manner which permits transgene transcription, translation, and/or expression in a host cell. The transgene can be used to correct or ameliorate gene deficiencies, which can include deficiencies in which normal genes are expressed at less than normal levels or at less than desired levels, or deficiencies in which the functional gene product is not expressed.

The term “polypeptide” as used herein refers to a polymer of amino acid residues, wherein the polymer can optionally contain a moiety or moieties that does not consist of amino acids (e.g., an alkyl group, an aralkyl group, an aryl group, or a synthetic polymer, such as, but not limited to a biocompatible polymer). The term applies to amino acid polymers in which one or more amino acid residue is an artificial chemical mimetic of a corresponding naturally occurring amino acid, as well as to naturally occurring amino acid polymers and non-naturally occurring amino acid polymers. The term “polypeptide” also includes a full-length gene product, including a biologically active gene product, and can also refer to a fragment of a full-length gene product, including a biologically active fragment. In some embodiments, the polypeptide comprises a sequence of SEQ ID NO:2, SEQ ID NO:4, SEQ ID NO:6, SEQ ID NO:8, or a sequence substantially identical thereto, or a fragment thereof, including a biologically active fragment. In some embodiments, the polypeptide is encoded by polynucleotide sequence comprising a sequence of SEQ ID NO:1, SEQ ID NO:3, SEQ ID NO:5, SEQ ID NO:7, or a sequence substantially identical thereto, or fragment thereof. In some embodiments, the term “biologically active” is meant to refer to a polypeptide or fragment of a polypeptide having an activity in the modulation of oxidative and/or glycolic metabolism in ischemic cells.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs are compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics are chemical compounds that have a structure that is different from the general chemical structure of an amino acid, but that function in a manner similar to a naturally occurring amino acid.

The term “amino acid residue” as used herein refers to a monovalent amino acid or derivative thereof. In some embodiments, the term “amino acid residue” refers to the group —NHC(R′)C(═O)OR″, wherein R′ is an amino acid side chain or protected derivative thereof and wherein R″ is H or a carboxylic acid protecting group, e.g., methyl.

“Pharmaceutically acceptable” refers to those carriers, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. Thus, in some embodiments, the presently disclosed compositions can be provided in formulations comprising the agent and a carrier that is pharmaceutically acceptable for use in humans.

In some embodiments, “treatment” or “treating” refers to an amelioration of disease or disorder, or at least one discernible symptom thereof. “Treatment” or “treating” also refers to an amelioration of at least one measurable physical parameter associated with a disease or disorder that is not necessarily discernible by the subject. “Treatment” or “treating” can also refer to inhibiting the progression of a disease or disorder either physically (e.g., stabilization of a discernible symptom), physiologically (e.g., stabilization of a physical parameter), or both. “Treatment” or “treating” also refers to delaying the onset of a disease or disorder, or a symptom thereof, or indeed preventing the disease or disorder, or a symptom thereof.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The term “operatively linked”, as used herein, refers to a functional combination between a promoter region and a nucleic acid molecule such that the transcription of the nucleic acid molecule is controlled and regulated by the promoter region. Techniques for operatively linking a promoter region to a nucleic acid molecule are known in the art.

The terms “vector”, “expression vector”, “expression cassette,” and “construct” are used interchangeably and refer to a nucleic acid molecule having nucleotide sequences that enable its replication in a host cell. A vector can also include nucleic acids to permit ligation of nucleotide sequences within the vector, wherein such nucleic acids are also replicated in a host cell. Representative vectors include plasmids and viral vectors. The term “vector” is also used to describe an expression construct, wherein the expression construct comprises a vector and a nucleic acid operatively inserted with the vector, such that the nucleic acid is expressed in the host cell.

Vectors can also comprise nucleic acids including expression control elements, such as transcription/translation control signals, origins of replication, polyadenylation signals, internal ribosome entry sites, promoters, enhancers, etc., wherein the control elements are operatively associated with a nucleic acid encoding a gene product. Many such sequences can be derived from commercially available vectors. See e.g., Sambrook & Russell (2001) Molecular Cloning: a Laboratory Manual, 3rd ed. Cold Spring Harbor Laboratory Press, Cold Spring Harbor, N.Y. and references cited therein.

The terms “cis-acting regulatory sequence” or “cis-regulatory motif” or “cis-regulatory element” or “response element”, as used herein, each refer to a nucleotide sequence within a promoter region that enables responsiveness to a regulatory transcription factor. Responsiveness can encompass a decrease or an increase in transcriptional output and is mediated by binding of the transcription factor to the DNA molecule comprising the response element.

The term “transcription factor” generally refers to a protein that modulates gene expression by interaction with the cis-regulatory element and cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene can be assayed when variably placed under the direction of a promoter region fragment.

“Ischemia” refers to inadequate blood flow to a cell, tissue, or organ, which typically results in hypoxia, a deficiency in the amount of oxygen, and in the cell, tissue, or organ's inability to meet demands for metabolism. Ischemia can be the result of a surgery or another trauma that includes the reduction of blood flow to a tissue or organ, or can be associated with another disease or condition, such as diabetes. The term “ischemic injury” as used herein can refer to an effect of, and/or damage caused by, ischemia in a cell, tissue, organ or subject. Ischemic injury can refer to one or more of tissue necrosis, myopathy (e.g., muscle weakness, loss of function or pain), fibrosis, or vascular deficiency.

### II. General Considerations

Normal cellular function requires the development and maintenance of a cellular energy charge (ATP/ADP ratio), which can be generated through numerous metabolic pathways. Loss of this energy charge results in an inability to continue required cell functions/processes and ultimately cell death. Under normal conditions, this cellular energy charge is primarily generated through oxidative metabolism within the mitochondria. Under acute and chronic ischemia (lack of oxygen), cells alter their metabolic pathways to maintain cellular functions and prevent cell death. For example, in the absence of oxygen, cells can alter their preferred energy pathway and produce energy through non-oxidative pathways, including glycolysis.

In some embodiments, the presently disclosed subject matter provides chemical and genetic “targets” that are capable of modulating ischemic cell bioenergetics. In some embodiments, the genetic target is Cox6a2, a striated muscle specific protein subunit of cytochrome c oxidase (ferrocytochrome c:O₂ oxidoreductase), otherwise referred to as Complex IV of the mitochondrial electron transport system. As described further hereinbelow, a loss of Cox6a2 protein expression during ischemia results in a dramatic reduction in the capacity for mitochondrial respiration and energy production, ultimately leading to the destruction and impaired regeneration of ischemic muscle cells. Thus, it appears that the loss of Cox6a2 can be a “trigger” for the progression of the ischemic pathology. Additionally, as also described further hereinbelow, overexpression of Cox6a2 rescues limb necrosis and skeletal myopathy through improved cellular bioenergetics and structural and functional maintenance of the mitochondrial electron transport system.

In some embodiments, the presently disclosed subject matter relates to the discovery that genetically or pharmacologically enhancing glycolytic metabolism is protective against hypoxia/ischemia. Particularly, in accordance with some embodiments of the presently disclosed subject matter, it has been uncovered that elevated expression of the enzyme 6-phosphofructo-2-kinase/fructose-2,6-bisphosphatase 3 (PFKFB3), a potent allosteric activator of glycolysis, prevents ischemic injury and improves cell survival, while small molecular inhibition of PFKFB3 severely exacerbates ischemic injury. Thus, in some embodiments, the genetic target is PFKFB3. Additionally, in accordance with some embodiments of the presently disclosed subject matter, the presently disclosed subject matter is also based on the findings of high-throughput hypoxic cell growth assays, which show that several small molecule compounds capable of reprogramming cell metabolism towards glycolysis can also improve cell growth/survival in hypoxia. The compounds include, for example, metform in, phenformin, biperiden hydrochloride, clemastine, meclizine, berberine chloride, vinpocetine, pimozide, and mefloquine.

Accordingly, in some embodiments, the presently disclosed subject matter provides genetic, chemical, and peptide compounds that can be protective against ischemic diseases including, but not limited to, peripheral arterial disease, stroke, and heart attacks.

In some aspects, the presently disclosed subject matter provides for targeting mitochondria, including but not limited to Complex IV and Cox6a2, through all feasible pharmacologic, genetic, or peptide/fragment delivery options for therapeutic treatment of ischemic pathology. This treatment is not limited to peripheral limb muscle, but can also include, for example cardiac muscle and diaphragm. Thus, an “agent adapted to modulate oxidative metabolism in ischemic cells” is meant to encompass any pharmacologic, genetic, or peptide/fragment option for therapeutic treatment of ischemic pathology modulation of oxidative metabolism, such as energy production metabolic activities by mitochondria. In some embodiments, the term “modulate oxidative metabolism” refers to an agent that can, for example, increase muscle cell mitochondrial respiration, maintain cellular energy change, and/or stimulate paracrine signals to expand the vascular network.

In some aspects, the presently disclosed subject matter provides for targeting glycolysis, including but not limited to PFKFB3 and other allosteric components of glycolysis, through all feasible pharmacologic, genetic, or peptide/fragment delivery options for therapeutic treatment of ischemic pathology. In some embodiments, the ischemic pathology is related the group including, but not limited to, peripheral limb muscle, diaphragm, cardiac muscle, brain/neurons, liver, kidney, bladder, and lungs. In some aspects, the presently disclosed subject matter provides for targeting glycolysis for therapeutic treatment of ischemic limb pathology. Thus, the term “modulate glycolytic metabolism” as used with regard to the term “modulate oxidative and/or glycolytic metabolism” herein can refer to an agent that can increase and/or otherwise enhances glycolytic metabolism.

In some embodiments, the presently disclosed subject matter provides methods of treating ischemia in a subject. In some embodiments, the method comprises administering to the subject a composition comprising an agent adapted to modulate oxidative and/or glycolytic metabolism in ischemic cells in the subject; and treating at least one symptom associated with the ischemia in the subject. For instance, in some embodiments, the symptom associated with ischemia is a symptom associated with ischemic injury, such as, but not limited to, tissue necrosis, myopathy, fibrosis, or vascular deficiency.

In some embodiments, the presently disclosed subject matter can provide, in some embodiments, a pharmaceutical composition for treating ischemia in a subject. In some embodiments, the composition comprises an agent adapted to modulate oxidative and/or glycolytic metabolism in ischemic cells in the subject, and a pharmaceutically acceptable excipient. In some embodiments, the composition can comprise one or more agents adapted to modulate oxidative metabolism in ischemic cells, one or more agents adapted to modulate glycolytic metabolism in ischemic cells, or both (i) one or more agents adapted to modulate oxidative metabolism in ischemic cells and (ii) one or more agents adapted to modulate glycolytic metabolism in ischemic cells. In some embodiments, the composition can comprise one or more agents adapted to modulate oxidative metabolism in ischemic cells. In some embodiments, the composition can comprise one or more agents adapted to modulate glycolytic metabolism in ischemic cells.

Thus, the presently disclosed subject matter provides an agent that is adapted to modulate oxidative metabolism in mitochondria in ischemic cells in the subject. In some embodiments, the agent is adapted to provide Cox6a2 to the ischemic cells in the subject. In some embodiments, the agent comprises a polynucleotide encoding a Cox6a2 polypeptide. In some embodiments, the polypeptide comprises a sequence of SEQ ID NO:2, SEQ ID NO:4, or a sequence substantially identical thereto. In some embodiments, the polynucleotide comprises a sequence of SEQ ID NO:1, SEQ ID NO:3, or a sequence substantially identical thereto. In some embodiments, the polynucleotide encoding the Cox6a2 polypeptide is operably coupled to a targeting vector capable of causing the expression of the Cox6a2 polypeptide in at least one of a muscle cell, fibroblast, stem cell, pericyte, and an endothelial cell. In some embodiments, the agent is an adeno-associated virus (AAV) vector particle comprising a polynucleotide encoding Cox6a2 and having at least about 70% sequence identity (e.g., at least 70%, 75%, 80%, 85%, 90%, 92%, 94%, 95%, 96%, 97%, 98%, or 99% A sequence identity) with SEQ ID NO: 1 or SEQ ID NO: 3, wherein said polynucleotide sequence is operably linked to a promoter.

In some embodiments, the agent of the presently disclosed subject matter is adapted to modulate glycolytic metabolism in ischemic cells in the subject. In some embodiments, the agent comprises one or more small molecule pharmacological agents. In some embodiments, the one or more small molecule pharmacological agents are selected from the group comprising metformin, phenformin, biperiden hydrochloride, clemastine, meclizine, berberine chloride, vinpocetine, pimozide, and mefloquine. In some embodiments, the agent is adapted to provide PFKFB3 to the ischemic cells in the subject. In some embodiments, the agent comprises a polynucleotide encoding a PFKFB3 polypeptide. In some embodiments, the polypeptide comprises a sequence of SEQ ID NO:6, SEQ ID NO:8, or a sequence substantially identical thereto. In some embodiments, the polynucleotide comprises a sequence of SEQ ID NO:5, SEQ ID NO:7, or a sequence substantially identical thereto. In some embodiments, the polynucleotide encoding the PFKFB3 polypeptide is operably coupled to a targeting vector capable of causing the expression of the PFKFB3 polypeptide in a tissue or cell type. In some embodiments, the cell type is at least one of a muscle cell, fibroblast, stem cell, pericyte, and endothelial cell. In some embodiments, the agent is an AAV vector particle comprising a polynucleotide encoding PFKFB3 and having at least about 70% sequence identity (e.g., at least 70%, 75%, 80%, 85%, 90%, 92%, 94%, 95%, 96%, 97%, 98%, or 99% A sequence identity) with SEQ ID NO: 5 or SEQ ID NO: 7, wherein said polynucleotide sequence is operably linked to a promoter.

In some embodiments, the composition comprising an agent adapted to modulate oxidative and/or glycolytic metabolism in ischemic cells in the subject comprises a liposome or nanoparticle, adapted to facilitate delivery of the agent in accordance with conventional techniques. In some embodiments, the agent is adapted to provide a Cox6a2 polypeptide thereof, a PFKFB3 polypeptide, or both a Cox6a2 polypeptide and a PFKFB3 polypeptide. In some embodiments, the polypeptide comprises a sequence of SEQ ID NO:2, SEQ ID NO:4, SEQ ID NO:6, SEQ ID NO:8, or a sequence substantially identical thereto. In some embodiments, the composition provides for delivery of the protective mitochondrial Complex IV Cox6a2 gene and/or PFKFB3 gene to ischemic cells or tissues. In some embodiments, the gene encodes a polypeptide comprising a sequence of SEQ ID NO:2, SEQ ID NO:4, SEQ ID NO:6, SEQ ID NO:8, or a sequence substantially identical thereto. In some embodiments, the gene comprises a sequence of SEQ ID NO:1, SEQ ID NO:3, SEQ ID NO:5, SEQ ID NO:7, or a sequence substantially identical thereto.

Delivery of Cox6a2 gene and/or PFKFB3 gene can be accomplished using any of a variety of different approaches, including but not limited to liposomal, nanoparticle, plasmid DNA recombinant adenovirus, recombinant adeno-associated virus (AAV), recombinant lentivirus, and combinations thereof. In a particular example, an AAV vector is employed for efficient and long-term expression of the Cox6a2 or PFKFB3 gene in the target ischemic skeletal muscle tissue. In some embodiments, the composition is adapted for administration by one or more of intramuscular injection, percutaneous injection, intraperitoneal injection, intravenous injection and oral consumption. Additional composition preparation and delivery techniques are disclosed in U.S. Pat. Nos. 9,719106; 9,610,363; 9,468,687; 9,309,534; 9,308,281; 9,267,123; 8,217,157; 7,906,111; and U.S. Patent Application Publication Nos.: 2018/0010136 and 2015/0020223, the entire contents of each of which are herein incorporated by reference in their entireties, as well as further hereinbelow.

In some embodiments, the ischemia in the subject comprises an ischemic injury. In some embodiments, the ischemic injury comprises tissue necrosis, myopathy, fibrosis, or vascular deficiency. In some embodiments, the ischemic injury is caused by one or more of peripheral arterial disease comprising intermittent claudication, critical limb ischemia, trauma, diabetes mellitus, and/or acute limb ischemia. In some embodiments, the ischemia is caused by one or more of peripheral artery disease, stroke, renal ischemia, myocardial infarction and ischemic limb pathology.

In some embodiments, the presently disclosed methods provide for the treatment of the most severe manifestations of peripheral arterial disease (PAD). PAD pathology is caused by atherosclerotic obstruction of the peripheral arteries and manifests as either anti-symptomatic, intermittent claudication (IC), or the more severe critical limb ischemia (CLI), which results in chronic rest pain and/or tissue necrosis. Within the ischemic limb muscle, mitochondria are well positioned to modulate the ischemic cellular response, as they are not only the principal site of cellular energy production, but also regulate cell death through apoptotic signaling and are the major regulator of the cellular redox charge.

The limb skeletal muscle thus represents a unique tissue in which to develop and implement effective therapies for CLI. In some embodiments, the presently disclosed subject matter is related to the role of the limb skeletal muscle mitochondria, and mitochondrial respiration, in the manifestation of CLI pathology in response to the decreased blood supply that occurs in PAD. Mitochondrial mechanisms by which limb muscle cells respond to ischemia and influence the limb vasculature in PAD are believed to represent a new strategy to prevent tissue loss and subsequent limb amputation in patients with CLI.

Thus, in some embodiments, the term “agent adapted to modulate oxidative metabolism in ischemic cells in the subject” is meant to encompass an agent that targets mitochondrial activity in cellular energy production, including the targeting of limb muscle mitochondria, including the activity of limb skeletal muscle mitochondria in energy production and redox homeostasis. By way of particular example, in some embodiments, the presently disclosed subject matter provides for the therapeutic targeting of limb muscle mitochondria, particularly mitochondrial Complex IV and Cox6a2, to prevent ischemic tissue loss and enhance muscle regeneration to alleviate morbidity/mortality outcomes with critical limb ischemia. Delivering Cox6a2 to ischemic muscle tissue improves limb muscle mitochondrial respiration and maintaining the cellular energy charge, thereby enhancing cellular survival and stimulating paracrine signals to expand and protect the vascular network to the ischemic limb to restore blood flow and prevent tissue loss. The presently disclosed subject matter can thus treat and/or protect skeletal muscle from developing any pathology that is the direct result of or a secondary effect of reduced blood flow (ischemia) whether it is a mild temporary form or a chronic more severe form of ischemia.

Additionally, the presently disclosed subject matter provides for the alleviation of cardiac ischemic myopathy and the loss of tissue or tissue functions in other diseases involving ischemia of acute or chronic natures. These diseases can include, but are not limited to, obesity, cardiac myopathy, myocardial infarction or congestive heart failure, neuronal ischemia (hemorrhagic or occlusive stroke), diabetes mellitus, congenital muscular dystrophies, limb-girdle muscular dystrophy (LGMD) syndromes, myofibrillar myopathies including but not limited to mutations in the DES, CRYAB, MYOT, LDB3, FLNC, and BAG3 genes, and myopathies with mitochondrial mutations or deletions which include: Kearns-Sayre syndrome (KSS), chronic progressive external ophthalmoplegia (CPEO), mitochondrial encephalomyopathy with lactic acidosis and stroke-like episodes (MELAS), myoclonic epilepsy with ragged-red fibers (MERRF), neurogenic weakness with ataxia and retinitis pigmentosa (NARP), mitochondrial recessive ataxia syndrome (MIRAS), or Leigh syndrome (LS).

Ischemic muscle cells demonstrate a rapid and drastic shift towards catabolic and apoptotic processes. See McClung et al., The American Journal of Pathology 2012, 180:2156-69. When coupled with the relative tolerance of vascular endothelial cells to the same insult, it appears that these cell types are individually programmed to respond in ways that preserve vascular integrity while sacrificing muscle cell homeostasis. Muscle's unique ability to regenerate or recover from wasting or traumatic injury, in part due to the availability of endogenous muscle stem cell-like progenitors including satellite cells and pericytes, supports the idea of evolutionary selection of muscle cell plasticity as a key protective mechanism against severe ischemia, facilitating survival until blood flow can be restored by angiogenesis and/or collateral vessel formation. Differentiating muscle cells secrete traditional vascular growth factors that act as both autocrine and paracrine factors to stimulate maturation in both endothelial and muscle cells, and represents a unique source of regenerative signals that can be harnessed to improve the local ischemic microenvironment and stimulate tissue survival. Limb skeletal muscle mitochondria are critical to energy production and redox homeostasis, and alterations or exacerbations in limb perfusion such as those induced by ischemia could contribute to alterations in mitochondrial content and/or function that dictate both the scale of muscle's degenerative myopathy and its regenerative potential. In some embodiments, the presently disclosed subject matter targets the limb muscle mitochondria to stimulate the survival of the tissue and prevention of morbidity/mortality outcomes.

In some embodiments, administering a composition comprising an agent adapted to modulate oxidative metabolism in ischemic cells, an agent adapted to modulate glycolytic metabolism in ischemic cells, or both an agent adapted to modulate oxidative metabolism in ischemic cells and an agent adapted to modulate glycolytic metabolism in ischemic cells can increase one or more of muscle fiber cross-sectional area, capillary density, muscle function, muscle regeneration, stem cell activity, vascular density, and vascular luminal diameter. For example, an increase in myotube diameter, change in myotube phenotype, change in contractile function, an increase in stem cell or satellite cell activity/myogenesis, an increase in mitochondrial number or respiratory function, an increase in autophagic flux, and decreased DNA fragmentation can be accomplished. In some embodiments, the administering of a composition comprising an agent adapted to modulate oxidative metabolism in ischemic cells, an agent adapted to modulate glycolytic metabolism in ischemic cells, or both an agent adapted to modulate oxidative metabolism in ischemic cells and an agent adapted to modulate glycolytic metabolism in ischemic cells can cause one or more of increased expression of vascular endothelial growth factor (VEGF), neuropilin (Nrp-1), vascular endothelial growth factor receptor 1 (Flt), vascular endothelial growth factor receptor 2 (Flk), myogenin, myoD, Tmem8c (myomaker) and muscle RING-finger protein 1 (MuRF-1), PGC1-alpha, opa1, Drp1, Mitofusion (Mfn) 1 or 2, and decreased in expression of myostatin.

In some embodiments, the presently disclosed subject matter can be used in conjunction with conventional techniques. Representative conventional techniques include surgical and percutaneous revascularization techniques.

In some embodiments, the presently disclosed subject matter can be used to treat and/or protect skeletal muscle from diseases or pathologies involving degeneration and regeneration as well as protection from developing any pathology that is the direct result of or a secondary effect of reduced blood flow that could contribute to some form of ischemia whether it is a mild temporary form or a chronic more severe form of ischemia. Thus, targeting metabolism (mitochondrial and glycolytic) has therapeutic potential that reaches beyond peripheral arterial disease. Although the Cox6a2 target is specific to muscle cells, PFKFB3 is expressed in all cells types, and improving metabolism will improve ischemic outcomes in multiple cell types (as shown in the Figures). Thus, the presently disclosed subject matter provides therapeutic options for stroke, myocardial infarction, kidney ischemia, etc.

### III. Polypeptide and Polynucleotide Agents

As described hereinabove, in some embodiments, the presently disclosed subject matter relates to genetic and peptide agents that are adapted to modulate oxidative and/or glycolytic metabolism in ischemic cells. When the agent adapted to modulate oxidative and/or glycolytic metabolism is a protein or polypeptide (e.g., the Cox6a2 or PFKFB3 proteins), it can be introduced into ischemic cells (e.g., ischemic muscle cells) directly, in accordance with conventional techniques, e.g., via formulation in a pharmaceutically acceptable carrier and subsequent injection. Additionally, a protein/polypeptide agent can be introduced into ischemic cells indirectly, by introducing into the cells a nucleic acid encoding the agent, in a manner permitting expression of the protein/polypeptide agent. The agent can be introduced into cells, in vitro or in vivo, using conventional procedures known in the art, including, without limitation, electroporation, DEAE dextran transfection, calcium phosphate transfection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, or any combination thereof. Recombinant viral vectors suitable for gene therapy include, but are not limited to, vectors derived from the genomes of such viruses as retrovirus, HSV, adenovirus, adeno-associated virus, Semiliki Forest virus, cytomegalovirus, lentivirus, and vaccinia virus.

In accordance with some embodiments of the presently disclosed subject matter, genes encoding proteins (e.g., Cox6a2 or PFKFB3) associated with improved cellular bioenergetics, the structural and functional maintenance of the mitochondrial electron transport system and/or enhanced glycolysis can be used as a tool to develop gene therapy compositions to be used in therapeutic applications to treat, prevent or minimize effects of ischemia and/or ischemic injury. In some embodiments, a gene therapy composition and method can be used in conjunction (i.e. before, during, after, or a combination thereof) with exogenous protein administration, the administration of a small molecule pharmacological agent that enhances glycolytic metabolism in an ischemic cell, and/or another pharmacological agent used to treat ischemia or an associated/related condition, e.g., stroke, myocardial infarction, PAD, CLI, IC, congestive heart failure, and diabetes.

The general strategy of gene therapy is the insertion of an introduced non-native sequence of DNA, e.g. a coding sequence for a polypeptide of interest, into a cell, tissue or organ of a subject, and in some instances incorporation into the subject's native DNA, in order to facilitate a biological change. For instance, by way of example and not limitation, the nucleic acid sequence SEQ ID NO: 3 coding for the Cox6a2 polypeptide having an amino acid sequence of SEQ ID NO: 4 can be introduced and expressed, e.g., constitutively or by induction, in a cell or tissue, e.g., a skeletal muscle cell or tissue, of a subject to thereby affect a change (e.g., an increase) in the expression and/or activity of Cox6a2 in the cell or tissue. This approach can be used with cells capable of being grown in culture in order to study the function of the nucleic acid sequence, as well as in vivo as a therapeutic strategy. General representative gene therapy methods are described, for example, in U.S. Pat. Nos. 5,279,833; 5,286,634; 5,399,346; 5,646,008; 5,651,964; 5,641,484; and 5,643,567, the contents of each of which are herein incorporated by reference.

Gene therapy methods and compositions of the presently disclosed subject matter are directed toward modulation of the expression and/or activity of any polypeptide of interest to thereby affect or modulate the biological activity of a polypeptide of interest and prevent or alleviate the symptoms of ischemia or ischemic injury, e.g., tissue necrosis, myopathy, fibrosis and/or vascular deficiency. In some embodiments, methods and compositions are provided for increasing the expression and/or activity of Cox6a2 and/or PFKFB3. In some embodiments, a gene therapy is provided for Cox6a2 expression in at least one of a muscle cell, a fibroblast, a stem cell, a pericyte and an endothelial cell. In some embodiments, a gene therapy is provided for PFKFB3 expression in at least one of a muscle cell, a fibroblast, a stem cell, a pericyte and an endothelia cell.

In some embodiments, gene therapy constructs and methods are provided to increase one or more of the group including, but not limited to, muscle fiber cross-sectional area, capillary density, muscle function, muscle regeneration, stem cell activity, vascular density, and vascular luminal diameter. In some embodiments, the gene therapy causes an increase in myotube diameter, a change in myotube phenotype, a change in contractile function, an increase in stem cell or satellite cell activity/myogenesis, an increase in mitochondrial number or respiratory function, an increase in autophagic flux, decreased DNA fragmentation, or combinations thereof. In some embodiments, the gene therapy causes one or more of increased expression of vascular endothelial growth factor (VEGF), neuropilin (Nrp-1), vascular endothelial growth factor receptor 1 (Flt), vascular endothelial growth factor receptor 2 (Flk), myogenin, myoD, Tmem8c (myomaker), muscle RING-finger protein 1 (MuRF-1), PGC1-alpha, opa1, Drp1, and Mitofusion (Mfn) 1 or 2 and/or decreased expression of myostatin.

III.A. Gene Therapy Constructs

In some embodiments the presently disclosed subject matter provides a gene therapy vehicle, delivery system or construct comprising a vector encoding a polypeptide of interest. The particular vector employed in accordance with the presently disclosed subject matter is not intended to be a limitation of the disclosed and claimed compositions and methods. Any suitable vector, construct or delivery vehicle as would be apparent to those of skill in the art upon a review of the instant disclosure can be used within the scope of the presently disclosed subject matter. The vector can be, for instance, a plasmid and/or a recombinant AAV viral vector. However, it will be readily understood that expression cassettes containing nucleic acid sequences as described herein can be engineered onto any number of vectors, including other viral vectors such as baculovirus, adenovirus, lenitviruses, retroviruses, and the like.

Thus, the vector for the agent of the presently disclosed subject matter can be a viral vector or a non-viral vector. Exemplary suitable viral vectors include, but are not limited to, adenoviruses, adeno-associated viruses (AAVs), self complementary AAV (scAAV; Buie et al., Invest Ophthalmol Vis Sci. 2010, 51:236-248), retroviruses, pseudotyped retroviruses, herpes viruses, vaccinia viruses, Semiliki forest virus, and baculoviruses. Exemplary suitable non-viral vectors include, but are not limited to, plasmids, water-oil emulsions, polethylene imines, dendrimers, micelles, microcapsules, liposomes, and cationic lipids. Polymeric carriers for gene therapy constructs can be used as described in Goldman et al. (Nat Biotechnol 1997, 15:462) and U.S. Pat. Nos. 4,551,482 and 5,714,166, the disclosures of which are incorporated herein by reference in their entireties. Where appropriate, two or more types of vectors can be used together. For example, a plasmid vector can be used in conjunction with liposomes. Provided in some embodiments of the presently disclosed subject matter is the use of an adenovirus or an AAV, as described further herein below.

Suitable methods for introduction of a gene therapy construct into cells include those described above, such as, but not limited to, direct injection into a cell or cell mass, particle-mediated gene transfer, electroporation, DEAE-Dextran transfection, liposome-mediated transfection, viral infection, and combinations thereof. A delivery method is selected based considerations such as the vector type, the toxicity of the encoded gene, the condition or tissue to be treated and the site of administration and/or treatment.

III.A.1. Viral Gene Therapy Vectors

Nucleic acid constructs of the presently disclosed subject matter can be incorporated into viral genomes by any suitable approach known in the art. In some embodiments, such incorporation can be performed by ligating the construct into an appropriate restriction site in the genome of the virus. Viral genomes can then be packaged into viral coats or capsids by any suitable procedure. In particular, any suitable packaging cell line can be used to generate viral vectors of the presently disclosed subject matter. In some embodiments, these packaging lines can complement the replication-deficient viral genomes of the presently disclosed subject matter, as they include, typically incorporated into their genomes, the genes which have been deleted from the replication-deficient genome. Thus, the use of packaging lines can allow viral vectors of the presently disclosed subject matter to be generated in culture.

Accordingly, in some embodiments, viral vectors of the presently disclosed subject matter can be disabled, e.g. replication-deficient. That is, they lack one or more functional genes required for their replication, which prevents their uncontrolled replication in vivo and avoids undesirable side effects of viral infection. In some embodiments, all of the viral genome is removed except for the minimum genomic elements required to package the viral genome incorporating the therapeutic gene into the viral coat or capsid. For example, in some embodiments it is desirable to delete all the viral genome except the Long Terminal Repeats (LTRs) or Invented Terminal Repeats (ITRs) and a packaging signal. In the cases of adenoviruses, deletions can be made in the E1 region and optionally in one or more of the E2, E3 and/or E4 regions. In the case of retroviruses, genes required for replication, such as env and/or gag/pol can be deleted. Deletion of sequences can be achieved by recombinant approaches, for example, involving digestion with appropriate restriction enzymes, followed by religation. Replication-competent self-limiting or self-destructing viral vectors can also be used.

In some embodiments the vector is an adenoviral vector. By way of example and not limitation, adenovirus titration and determination of infectivity are known in the art. Also by way of example and not limitation, design and incorporation of nucleic acid constructs and expression cassettes into viral vectors, as well as construction of adeno-associated viral (AAV) gene therapy constructs are known in the art.

More particularly, for use in producing a viral vector (e.g., a recombinant (r) AAV), the expression cassette can be carried on any suitable vector, e.g., a plasmid, which is delivered to a packaging host cell. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences can include promoters, translation leader sequences, introns, polyadenylation recognition sequences, RNA processing sites, effector binding sites and stem-loop structures. Suitable transfection techniques and packaging host cells are known and/or can be readily designed by one of skill in the art.

Examples of constitutive promoters suitable for controlling expression of the transgenes include, but are not limited to, chicken β-actin (CB) promoter, human cytomegalovirus (CMV) promoter, the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (see Scharfmann et al., Proc. Natl. Acad. Sci. USA 1991, 88:4626-4630), adenosine deaminase promoter, phosphoglycerol kinase (PGK) promoter, pyruvate kinase promoter phosphoglycerol mutase promoter, the β-actin promoter (see Lai et al., Proc. Natl. Acad. Sci. USA 1989, 86: 10006-10010), the long terminal repeats (LTR) of Moloney Leukemia Virus and other retroviruses, the thymidine kinase promoter of Herpes Simplex Virus and other constitutive promoters known to those of skill in the art.

Examples of tissue- or cell-preferential promoters suitable for use in the presently disclosed subject matter include, but are not limited to, endothelin-I (ET-I) and Flt-I, which are for endothelial cells, FoxJ1 (that targets ciliated cells), human thyroxine binding globulin (TBG) and alpha-1 anti-trypsin (A1AT) for liver, troponin and T (TnT) for heart, clara cell 10 (CC10), surfactant protein C (SPC) and FoxJ1 for heart; synapsin, tyrosine hydroxylase, CaMKII (Ca2+/calmodulin-dependent protein kinase) for central nervous system/brain, insulin and elastase-I for pancrease, Ap2 and adiponector for adipocyte, desmin and MHC for muscle, and VMD for retina. Still others are known in the art.

Inducible promoters suitable for controlling expression of the transgene include promoters responsive to exogenous agents (e.g., pharmacological agents) or to physiological cues. These response elements include, but are not limited to a hypoxia response element (HRE) that binds hypoxia-inducible factor 1-alpha (HIF-1α) and HIF-1beta (HIF-1β), a metal-ion response element such as described by Mayo et al. (Cell 1982, 29:99-108); Brinster et al. (Nature 1982, 296:39-42) and Searle et al. (Mol. Cell. Biol. 1985, 5:1480-1489); or a heat shock response element such as described by Nouer et al. (in: Heat Shock Response, ed. Nouer, L., CRC, Boca Raton, Fla., ppl 67-220, (1991)).

Still other promoter systems can include response elements including but not limited to a tetracycline (tet) response element (such as described by Gossen & Bujard (Proc. Natl. Acad. Sci. USA 1992, 89:5547-551); or a hormone response element such as described by Lee et al. (Nature 1981, 294:228-232); Hynes et al. (Proc. Natl. Acad. Sci. USA 1981, 78:2038-2042); Klock et al. (Nature 1987, 329:734-736); and Israel & Kaufman (Nucl. Acids Res. 1987, 17:2589-2604), as well as other inducible promoters known in the art.

In some embodiments, the transgene sequence includes a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), enhanced GFP (EGFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal can be measured visually by color or light production in a luminometer.

Methods for generating and isolating AAVs suitable for use as vectors are known in the art. See generally, e.g., Grieger & Samulski, Adv. Biochem. Engin/Biotechnol. 2005, 99: 119-145; Buning et al., J. Gene Med. 2008, 10:717-733; and the references cited below, each of which is incorporated herein by reference in its entirety. An AAV viral vector is an AAV DNase-resistant particle having an AAV protein capsid into which is packaged nucleic acid sequences for delivery to target cells. In some embodiments, the AAV sequences on the expression cassette comprise only minimal AAV sequences to avoid the risk of replication. In some embodiments, the minimal AAV sequences include the AAV inverted terminal repeat sequences (ITR). The combination of the transgene, promoter/enhancer, and 5′ and 3′ AAV ITRs can be referred to as a “minigene” for ease of reference herein. The design of such a minigene can be made by resort to conventional techniques. In one embodiment, the 5′ ITR and the 3′ ITR are the minimal AAV sequences required in cis in order to express a transgene encoded by a nucleic acid sequence packaged in the AAV capsid. Typically, the ITRs flank the coding sequence for a selected gene product. In some embodiments, the AAV vector contains AAV 5′ and 3′ ITRs, which can be of the same AAV origin as the capsid. In some embodiments, the AAV vector contains AAV 5′ and 3′ ITRs of a different AAV origin (to produce an AAV pseudotype). In some embodiments, the coding sequences for the replication (rep) and/or capsid (cap) are removed from the AAV genome and supplied in trans or by a packaging cell line in order to generate the AAV vector.

An AAV capsid is composed of 60 capsid protein subunits, VP1, VP2, and VP3, that are arranged in an icosahedral symmetry in a ratio of approximately 1:1:10 to 1:1:20, depending upon the selected AAV. The capsid type can play a role in tissue specificity. The sequences of a variety of AAV have been described, as have methods of generating vectors having the AAV capsids. Examples of AAV which can be selected as sources for capsids of AAV viral vectors (DNase resistant viral particles) include, e.g., AAV1, AAV2, AAV3, AAV4, AAV5, AAV6, AAV6.2, AAV7, AAV8, AAV9, rh10, AAVrh64R1, AAVrh64R2, rh8. See, e.g., U.S. Published Patent Application No. 2007/0036760; U.S. Published Patent Application No. 2009/0197338; and EP 1310571, the disclosures of each of which are incorporated herein by reference in their entireties. See also, WO 2003/042397 (AAV7 and other simian AAV), U.S. Pat. No. 7,790,449 and U.S. Pat. No. 7,282,199 (AAV8), WO 2005/033321 and U.S. Pat. No. 7,906,111 (AAV9), and WO 2006/110689. These documents also describe other AAV which can be selected for generating AAV and are also incorporated by reference in their entireties.

The minigene, rep sequences, cap sequences, and helper functions required for producing the rAAV of the presently disclosed subject matter can be delivered to the packaging host cell in the form of any genetic element which transfer the sequences carried thereon. The selected genetic element can be delivered by any suitable method, including those described herein. The methods used to construct any embodiment of the presently disclosed subject matter are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the presently disclosed subject matter. See, e.g., K. Fisher et al, J. Virol. 1993, 70:520-532 and U.S. Pat. No. 5,478,745, incorporated herein by reference in its entirety.

Unless otherwise specified, the AAV ITRs, and other selected AAV components described herein, can be readily selected from among any AAV. Further, more than one AAV source can provide elements to an AAV vector. For example, as described above, a pseudotyped AAV can contain ITRs from a source which differs from the source of the AAV capsid. Additionally or alternatively, a chimeric AAV capsid can be utilized. Still other AAV components can be selected. Sources of such AAV sequences are described herein and can also be isolated or obtained from academic, commercial, or public sources (e.g., the American Type Culture Collection, Manassas, Va.). Alternatively, the AAV sequences can be obtained through synthetic or other suitable means by reference to published sequences such as are available in the literature or in databases such as, e.g., GENBANK®, PUBMED®, or the like.

The AAV vectors can be suspended in a physiologically compatible carrier for administration to a human or non-human mammalian patient. Suitable carriers can be readily selected by one of skill in the art in view of the route of delivery. For example, one suitable carrier includes saline, which can be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the presently disclosed subject matter. Optionally, the compositions of the presently disclosed subject matter can contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

III.A.2. Plasmid Gene Therapy Vectors

In some embodiments, a therapeutic gene can be encoded by a naked plasmid. The toxicity of plasmid DNA is generally low and large-scale production is relatively easy. Plasmid transfection efficiency in vivo encompasses a multitude of parameters, such as the amount of plasmid, time between plasmid injection and electroporation, temperature during electroporation, and electrode geometry and pulse parameters (field strength, pulse length, pulse sequence, etc.). The methods disclosed herein can be optimized for a particular application by methods known to one of skill in the art, and the presently disclosed subject matter encompasses such variations. See, e.g., Heller et al., FEBS Lett 1996, 389:225-228; Vicat et al., Hum Gene Ther 2000, 11:909-916; Miklavcic et al., Biophys J 1998, 74:2152-2158.

III.A.3. Liposomes

The presently disclosed subject matter also provides for the use of gene therapy constructs comprising liposomes. Liposomes can be prepared by any of a variety of techniques that are known in the art. See, e.g., Betageri et al., Liposome Drug Delivery Systems, Technomic Publishing, Lancaster (1993); Gregoriadis, ed., Liposome Technology, CRC Press, Boca Raton, Fla. (1993); Janoff, ed., Liposomes: Rational Design, M. Dekker, New York, N.Y. (1999); Lasic & Martin, Stealth Liposomes, CRC Press, Boca Raton, Fla. (1995); Nabel, “Vectors for Gene Therapy” in Current Protocols in Human Genetics on CD-ROM, John Wiley & Sons, New York, N.Y. (1997); and U.S. Pat. Nos. 4,235,871; 4,551,482; 6,197,333; and 6,132,766. Temperature-sensitive liposomes can also be used, for example THERMOSOMES™ as disclosed in U.S. Pat. No. 6,200,598. Entrapment of an active agent within liposomes of the presently disclosed subject matter can also be carried out using any conventional method in the art. In preparing liposome compositions, stabilizers such as antioxidants and other additives can be used.

Other lipid carriers can also be used in accordance with the presently disclosed subject matter, such as lipid microparticles, micelles, lipid suspensions, and lipid emulsions. See, e.g., Labat-Moleur et al., Gene Therapy 1996, 3:1010-1017; and U.S. Pat. Nos. 5,011,634; 6,056,938; 6,217,886; 5,948,767; and 6,210,707.

III.B. Constructs for Cox6a2 and PFKFB3

In some embodiments, the methods and compositions of the presently disclosed subject matter employ a gene therapy construct comprising a coding sequence (also referred to herein as a “nucleic acid molecule” or “therapeutic gene”) for a polypeptide of interest (also referred to herein as a “therapeutic polypeptide”). Thus, the terms “polypeptide of interest” and “therapeutic polypeptide” are used interchangeably and can refer to peptides whose concentration and/or activity in vivo modulate oxidative and/or glycolytic metabolism. In some embodiments a coding sequence for a polypeptide of interest corresponds to a gene that encodes the polypeptide of interest., e.g., Cox6a2 or PFKFB3. In some embodiments, a coding sequence for a polypeptide of interest corresponds to a gene of Cox6a2 or PFKFB3.

In some embodiments, a gene therapy construct is provided wherein the construct increases the levels of the polypeptide of interest, also referred to as a “therapeutic product”, in tissues or cells of interest. Thus, in some embodiments, the gene therapy construct is a targeting vector, comprising, for example, a constitutive, inducible or tissue- or cell-specific promoter operably linked to the gene encoding the polypeptide of interest. For instance, if expression in skeletal muscle is desired, a promoter active in muscle can be used. These include, but are not limited to, the promoters from genes encoding skeletal P-actin, myosin light chain 2A, dystrophin, muscle creatine kinase, as well as synthetic muscle promoters with activities higher than naturally-occurring promoters (see Li et al., Nat. Biotech. 1999, 17:241-245). Other examples of tissue- or cell-preferential promoters include, but are not limited to, endothelin-I (ET-I) and Flt-I, which are for endothelial cells, FoxJ1 (that targets ciliated cells), human thyroxine binding globulin (TBG) and alpha-1 anti-trypsin (A1AT) for liver, troponin and T (TnT) for heart, clara cell 10 (CC10), surfactant protein C (SPC) and FoxJ1 for heart; synapsin, tyrosine hydroxylase, CaMKII (Ca2+/calmodulin-dependent protein kinase) for central nervous system/brain, insulin and elastase-I for pancrease, Ap2 and adiponector for adipocyte, desmin and MHC for muscle, and VMD for retina. Still others are known in the art. See e.g., U.S. Pat. No. 9,719,106, incorporated herein by reference in its entirety.

In some embodiments, a gene therapy construct is provided that comprises a coding sequence comprising a nucleotide sequence of any of odd numbered SEQ ID NOs: 1-7; or a nucleic acid molecule comprising a nucleotide sequence substantially identical to any of odd numbered SEQ ID NOs: 1-7. In some embodiments, a gene therapy construct of the presently disclosed subject matter comprises a coding sequence comprising a nucleotide sequence that is 70%, 75%, 80%, 85%, 90%, 91%, 92%, 93%, 94%, 95%, 96%, 97%, 98% or 99% identical to any of odd numbered SEQ ID NOs: 1-7.

In some embodiments a gene therapy construct is provided that comprises a coding sequence comprising a nucleotide sequence encoding a polypeptide of any of even numbered SEQ ID NOs: 2-8; or a nucleic acid molecule comprising a nucleotide sequence encoding a polypeptide substantially identical to any of even numbered SEQ ID NOs: 2-8. In some embodiments, a gene therapy construct of the presently disclosed subject matter comprises a coding sequence comprising a nucleotide sequence encoding a polypeptide that is 70%, 75%, 80%, 85%, 90%, 91%, 92%, 93%, 94%, 95%, 96%, 97%, 98% or 99% identical to any of even numbered SEQ ID NOs: 2-8.

In some embodiments a gene therapy construct comprises a Cox6a2 nucleic acid molecule comprising a nucleotide sequence of SEQ ID NO: 1; or a nucleic acid molecule comprising a nucleotide sequence substantially identical of SEQ ID NO: 1. In some embodiments a gene therapy construct comprises a Cox6a2 nucleic acid molecule comprising a nucleotide sequence of SEQ ID NO: 3; or a nucleic acid molecule comprising a nucleotide sequence substantially identical of SEQ ID NO: 3. In some embodiments a gene therapy construct comprises a nucleic acid molecule encoding a Cox6a2 polypeptide comprising an amino acid sequence of SEQ ID NO: 2; a polypeptide comprising an amino acid sequence substantially identical to SEQ ID NO: 2; or a polypeptide that is a biological equivalent of or having a substantially similar biological activity of SEQ ID NO: 2. In some embodiments a gene therapy construct comprises a nucleic acid molecule encoding a Cox6a2 polypeptide comprising an amino acid sequence of SEQ ID NO: 4; a polypeptide comprising an amino acid sequence substantially identical to SEQ ID NO: 4; or a polypeptide that is a biological equivalent of or having a substantially similar biological activity of SEQ ID NO: 4. In some embodiments a gene therapy construct comprises a PFKFB3 nucleic acid molecule comprising a nucleotide sequence of SEQ ID NO: 5; or a nucleic acid molecule comprising a nucleotide sequence substantially identical of SEQ ID NO: 5. In some embodiments a gene therapy construct comprises a PFKFB3 nucleic acid molecule comprising a nucleotide sequence of SEQ ID NO: 7; or a nucleic acid molecule comprising a nucleotide sequence substantially identical of SEQ ID NO: 7. In some embodiments a gene therapy construct comprises a nucleic acid molecule encoding a PFKFB3 polypeptide comprising an amino acid sequence of SEQ ID NO: 6; a polypeptide comprising an amino acid sequence substantially identical to SEQ ID NO: 6; or a polypeptide that is a biological equivalent of or having a substantially similar biological activity of SEQ ID NO: 6. In some embodiments a gene therapy construct comprises a nucleic acid molecule encoding a PFKFB3 polypeptide comprising an amino acid sequence of SEQ ID NO: 8; a polypeptide comprising an amino acid sequence substantially identical to SEQ ID NO: 8; or a polypeptide that is a biological equivalent of or having a substantially similar biological activity of SEQ ID NO: 8. Optionally, a polypeptide encoded by a gene therapy construct of the presently disclosed subject matter displays one or more biological properties of a naturally occurring Cox6a2 or PFKFB3 polypeptide. The biological properties of a Cox6a2 or PFKFB3 polypeptide can be assessed using methods described in Examples herein below.

The term “substantially identical”, as used herein to describe a degree of similarity between nucleotide sequences, refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using one of the following sequence comparison algorithms or by visual inspection. In one embodiment, the substantial identity exists in nucleotide sequences of at least about 100 residues, in another embodiment in nucleotide sequences of at least about 150 residues, and in still another embodiment in nucleotide sequences comprising a full length sequence. The term “full length”, as used herein to refer to a complete open reading frame (ORF) encoding, for example, a gene of interest polypeptide. The term “full length” also encompasses a non-expressed sequence, for example a promoter or an inverted terminal repeat (ITR) sequence.

In one aspect, polymorphic sequences can be substantially identical sequences. The term “polymorphic” refers to the occurrence of two or more genetically determined alternative sequences or alleles in a population. An allelic difference can be as small as one base pair. In another aspect, substantially identical sequences can comprise mutagenized sequences, including sequences comprising silent mutations. A mutation can comprise a single base change.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “‘target” is a test nucleic acid molecule, often found within a heterogeneous population of nucleic acid molecules. A “target sequence” is synonymous with a “test sequence”.

In one embodiment, a nucleotide sequence employed for hybridization studies or assays includes probe sequences that are complementary to or mimic at least an about 14 to 40 nucleotide sequence of a nucleic acid molecule of the presently disclosed subject matter. In one embodiment, probes comprise 14 to 20 nucleotides, or even longer where desired, such as 30, 40, 50, 60, 100, 200, 300, or 500 nucleotides or up to the full length of any one of odd numbered SEQ ID NOs: 1-7. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook & Russell, 2001, for a description of SSC buffer. Often, a high stringency wash is preceded by a low stringency wash to remove background probe signal. An example of medium stringency wash conditions for a duplex of more than about 100 nucleotides is 15 minutes in 1×SSC at 45° C. An example of low stringency wash for a duplex of more than about 100 nucleotides is 15 minutes in 4× to 6×SSC at 40° C. For short probes (e.g., about 10 to 50 nucleotides), stringent conditions typically involve salt concentrations of less than about 1 M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the presently disclosed subject matter: in one embodiment a probe nucleotide sequence hybridizes to a target nucleotide sequence in 7% sodium dodecyl sulfate (SDS), 0.5 M NaPO₄, 1 mM EDTA at 50° C. followed by washing in 2×SSC, 0.1% SDS at 50° C.; in another embodiment, a probe and target sequence hybridize in 7% SDS, 0.5 M NaPO₄, 1 mM EDTA at 50° C. followed by washing in 1×SSC, 0.1% SDS at 50° C.; in another embodiment, a probe and target sequence hybridize in 7% SDS, 0.5 M NaPO₄, 1 mM EDTA at 50° C. followed by washing in 0.5×SSC, 0.1% SDS at 50° C.; in another embodiment, a probe and target sequence hybridize in 7% SDS, 0.5 M NaPO₄, 1 mM EDTA at 50° C. followed by washing in 0.1×SSC, 0.1% SDS at 50° C.; in another embodiment, a probe and target sequence hybridize in 7% SDS, 0.5 M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code (e.g., wherein the codons GCA, GCC, GCG, and GCU are functionally equivalent and each encode alanine).

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook & Russell, Molecular Cloning: a Laboratory Manual, 3rd ed. Cold Spring Harbor Laboratory Press, Cold Spring Harbor, N.Y. (2001); Silhavy et al., Experiments with Gene Fusions, Cold Spring Harbor Laboratory, Cold Spring Harbor, N.Y. (1984); Glover & Hames, DNA Cloning: A Practical Approach, 2nd ed. IRL Press at Oxford University Press, Oxford/New York (1995); Ausubel, Short Protocols in Molecular Biology, 3rd ed. Wiley, N.Y. (1995).

The term “substantially identical” in the context of two or more polypeptide sequences is measured as polypeptide sequences having in one embodiment at least about 35%, in another embodiment at least about 45%, in another embodiment 45-55%, and in another embodiment 55-65% of identical or functionally equivalent amino acids. In yet another embodiment, “substantially identical” polypeptides will have at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99% identical or functionally equivalent amino acids.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites. See, for example, Barton, Acta Crystallogr D Biol Crystallogr 2000, 54:1139-1146; Sagi et al., Bioinformatics, 1999, 15(6):521-522, Henikoff et al., Electrophoresis 2000, 21(9):1700-1706; and Huang et al., Symp Biocomput 2000, 230-241.

Substantially identical proteins also include proteins comprising an amino acid sequence comprising amino acids that are functionally equivalent to amino acids of a reference polypeptide. The term “functionally equivalent” in the context of amino acid sequences is known in the art and is based on the relative similarity of the amino acid side-chain substituents. See Henikoff and Henikoff, Adv Protein Chem 2000, 54:73-97. Relevant factors for consideration include side-chain hydrophobicity, hydrophilicity, charge, and size. For example, arginine, lysine, and histidine are all positively charged residues; alanine, glycine, and serine are all of similar size; and phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids can be substituted for other amino acids having a similar hydropathic index or score and still retain a similar biological activity. The substitution of amino acids whose hydropathic indices are in one embodiment within ±2 of the original value, in another embodiment within ±1 of the original value, and in yet another embodiment within ±0.5 of the original value are chosen in making changes based upon the hydropathic index.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

The substitution of amino acids whose hydrophilicity values are in one embodiment within ±2 of the original value, in another embodiment within ±1 of the original value, and in yet another embodiment within ±0.5 of the original value are chosen in making changes based upon similar hydrophilicity values.

The term “substantially identical” also encompasses polypeptides that are biologically functional equivalents. The term “functional” includes a biological activity of a peptide of the presently disclosed subject matter. By way of example and not limitation, a biological functional equivalent of a Cox6a2 or PFKFB3 polypeptide is a peptide having an activity substantially the same as that of a native Cox6a2 or PFKFB3 polypeptide. Representative methods for assessing Cox6a2 and PFKFB3 polypeptide function are described in the Examples. When used to describe a polypeptide encoded by a gene of interest, the term “functional” refers to any function desirably provided by the gene of interest. The presently disclosed subject matter also provides functional protein fragments of Cox6a2 or PFKFB3 family members or a gene product of interest. Such functional portion need not comprise all or substantially all of the amino acid sequence of a native Cox6a2 or PFKFB3 or polypeptide encoded by a gene of interest.

The presently disclosed subject matter also includes functional polypeptide sequences that are longer sequences than that of a native Cox6a2 or PFKFB3 family member or polypeptide of interest. For example, one or more amino acids can be added to the N-terminus or C-terminus of a therapeutic polypeptide. Methods of preparing such proteins are known in the art.

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

In some embodiments, the term “substantially identical” in regards to a nucleotide or polypeptide sequence means that a particular sequence varies from the sequence of a naturally occurring sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, for example by the local homology algorithm of Smith and Waterman (Adv Appl Math 1981, 2, 482); by the homology alignment algorithm of Needleman and Wunsch (J Mol Biol 1970, 48:443); by the search for similarity method of Pearson and Lipman (PNAS 1988, 85:2444-2448); by computerized implementations of these algorithms (GAP, BESTFIT, FASTA, and TFASTA in the Wisconsin Genetics Software Package, available from Accelrys Inc., San Diego, Calif., United States of America), or by visual inspection. See generally, Ausubel, Short Protocols in Molecular Biology, Third Edition, Wiley, N.Y. (1995).

An exemplary algorithm for determining percent sequence identity and sequence similarity is the BLAST algorithm, which is described in Altschul et al. (J Mol Biol 1990, 215: 403-410). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix. See Henikoff and Henikoff, Adv Protein Chem 2000, 54:73-97.

In addition to calculating percent sequence identity, the BLAST algorithm also performs a statistical analysis of the similarity between two sequences. See e.g., Karlin and Altschul, PNAS 1993, 90:5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

### IV. Pharmaceutical Compositions

In some embodiments, the presently disclosed subject matter provides pharmaceutical compositions for treating ischemia. The compositions can comprise a pharmaceutically acceptable excipient (which can also be referred to as a carrier or diluent) and an agent as described herein adapted to modulate oxidative and/or glycolytic metabolism. In some embodiments, a pharmaceutical composition can comprise one or more gene therapy constructs produced in accordance with the presently disclosed subject matter.

IV.A. Carriers

In some embodiments, a pharmaceutical composition can contain a pharmaceutically acceptable carrier or excipient, for administration of the agent (e.g., the gene therapy construct). In some embodiments, the carrier or excipient is pharmaceutically acceptable for use in humans. In some embodiments, the carrier or excipient is acceptable for use in animals (e.g., in veterinary settings). In some embodiments, suitable carriers can be large, slowly metabolized macromolecules such as proteins, polypeptides, liposomes, polysaccharides, polylactic acids, polyglycolic acids, polymeric amino acids, ammo acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonate and benzoates.

Pharmaceutically acceptable carriers in therapeutic compositions can additionally contain liquids such as water, saline, glycerol and ethanol. Additionally, auxiliary substances, such as wetting or emulsifying agents or pH buffering substances, can be present in such compositions. Such carriers enable the pharmaceutical compositions to be formulated for administration to the patient.

The compositions of the presently disclosed subject matter can further comprise a carrier to facilitate composition preparation and administration. Any suitable delivery vehicle or carrier can be used, including but not limited to a microcapsule, for example a microsphere or a nanosphere (Manome et al., Cancer Res 1994, 54:5408-5413; Saltzman & Fund, Adv Drug Deliv Rev 1997, 26:209-230), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al. Cancer Res 1997, 57:1447-1451 and U.S. Pat. Nos. 4,551,482, 5,714,166, 5,510,103, 5,490,840, and 5,855,900), and a polysome (U.S. Pat. No. 5,922,545).

IV.B. Formulations

Suitable formulations of pharmaceutical compositions of the presently disclosed subject matter include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS in the range of in some embodiments 0.1 to 10 mg/ml, in some embodiments about 2.0 mg/ml; and/or mannitol or another sugar in the range of in some embodiments 10 to 100 mg/ml, in some embodiments about 30 mg/ml; and/or phosphate-buffered saline (PBS). Any other agents conventional in the art having regard to the type of formulation in question can be used.

Pharmaceutical compositions of the presently disclosed subject matter can have a pH between 5.5 and 8.5, preferably between 6 and 8, and more preferably about 7. The pH can be maintained by the use of a buffer. The composition can be sterile and/or pyrogen free. The composition can be isotonic with respect to humans. Pharmaceutical compositions of the presently disclosed subject matter can be supplied in hermetically-sealed containers.

IV.C. Subjects

The subject treated in the presently disclosed subject matter is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which treatment of ischemic conditions or treatment or prevention of ischemic injury is desirable, particularly agricultural and domestic mammalian species.

The methods of the presently disclosed subject matter are particularly useful in the treatment of warm-blooded vertebrates. Thus, the presently disclosed subject matter concerns mammals and birds.

More particularly, provided herein is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, provided herein is the treatment of livestock, including, but not limited to, domesticated swine (pigs and hogs), ruminants, horses, poultry, and the like.

In some embodiments, the subject to be treated in accordance with the presently disclosed subject matter is a subject in need of treatment for ischemia. In some embodiments, a subject in need of treatment for ischemia comprises a subject suffering from, for example, stroke, myocardial infarction, ischemic limb pathology, peripheral arterial disease (PAD), including PAD comprising intermittent claudication (IC) or critical limb ischemia (CLI), trauma (e.g., trauma resulting in and/or caused by loss of blood flow), diabetes mellitus, and/or acute limb ischemia.

IV. D. Administration

Suitable methods for administration of a composition comprising an agent adapted to modulate oxidative and/or glycolytic metabolism, including compositions comprising a gene therapy construct of the presently disclosed subject matter, include but are not limited to intravenous injection, intramuscular injection, percutaneous injection, subcutaneous injection, intraperitoneal injection, or oral consumption. In some embodiments the compositions of the presently disclosed subject matter are administered via intramuscular injection. Alternatively, a composition of the presently disclosed subject matter can be deposited at a site in need of treatment in any other manner appropriate for the condition to be treated or the target site. In some embodiments, the particular mode of administering a therapeutic composition of the presently disclosed subject matter depends on various factors, including the distribution and abundance of cells to be treated, the composition (e.g., vector) employed, additional tissue- or cell-targeting features of the composition (e.g., vector), and mechanisms for metabolism or removal of the composition from its site of administration.

IV. E. Dose

An effective dose of a composition of the presently disclosed subject matter is administered to a subject in need thereof. The terms “therapeutically effective amount”, “therapeutically effective dose”, “effective amount’, “effective dose” and variations thereof are used interchangeably herein and refer to an amount of a therapeutic composition of the presently disclosed subject matter sufficient to produce a measurable response (e.g. increased muscle function or muscle regeneration in a subject being treated or increased respiratory function in a suitable cell model). Actual dosage levels of the compositions (e.g., the gene therapy constructs, and in some instances the therapeutic genes expressed by the gene therapy constructs), can be varied so as to administer an amount that is effective to achieve the desired therapeutic response for a particular subject. By way of example and not limitation, in some embodiments the gene therapy constructs of the presently disclosed subject matter can be administered at dose ranging from 5×10⁸ to 1×10¹⁰ virus genomes (vg), which would correspond to 2×10⁸ to 5×10⁹ infectious units (IFU).

With regard to small molecule compound agents of the presently disclosed subject matter, as a general proposition, a dosage from about 0.1 to about 50 mg/kg can have therapeutic efficacy, with all weights being calculated based upon the weight of the small molecule compound agent, including the cases where a salt is employed. Toxicity concerns at the higher level can restrict intravenous dosages to a lower level, such as up to about 10 mg/kg, with all weights being calculated based on the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. In some embodiments, dosages can be from about 1 μmol/kg to about 50 μmol/kg, or, optionally, between about 22 μmol/kg and about 33 μmol/kg of the compound for intravenous or oral administration. For small molecule agents, toxicity and therapeutic efficacy of compositions can be determined by standard pharmaceutical procedures in cell cultures or experimental animals (e.g., for determining the LD₅₀ (the dose lethal to 50% of the population) and the ED₅₀ (the dose therapeutically effective in 50% of the population)). The dose ratio between toxic and therapeutic effects is the therapeutic index and it can be expressed as the ratio LD₅₀/ED₅₀.

In some embodiments, the dosage of a gene therapy construct can be varied to achieve a desired level of Cox6a2 or PFKFB3 expression and/or activity in a subject. In some embodiments, a dosage of gene therapy construct of the presently disclosed subject matter can be optimized to treat, prevent or reverse ischemic injury in a subject, including but not limited to tissue necrosis, myopathy, fibrosis, or vascular deficiency.

In some embodiments, the quantity of a therapeutic composition of the presently disclosed subject matter administered to a subject will depend on a number of factors including but not limited to the subject's size, weight, age, the target tissue or organ, the route of administration, the condition to be treated, and the severity of the condition to be treated.

In some embodiments the selected dosage level will depend upon the activity of the therapeutic composition, the route of administration, combination with other drugs or treatments, the severity of the condition being treated, and the condition and prior medical history of the subject being treated. However, upon a review of the instant disclosure, it is within the skill of the art to consider these factors in optimizing an appropriate dosage, including for example starting doses of the compound at levels lower than required to achieve the desired therapeutic effect and to gradually increase the dosage until the desired effect is achieved. Moreover, upon review of the instant disclosure one of ordinary skill in the art can tailor the dosages to an individual subject by making appropriate adjustments or variations, as well as evaluation of when and how to make such adjustments or variations, as is routine to those of ordinary skill in the art.

The potency of a therapeutic composition can vary, and therefore a “therapeutically effective” amount can vary. However, using the assay methods described herein below, one skilled in the art can readily assess the potency and efficacy of a gene therapy construct of presently disclosed subject matter and adjust the therapeutic regimen accordingly. For example, polypeptide (e.g., Cox6a2 or PFKFB3) expression and/or an activity related to cellular bioenergetics and/or muscle function or regeneration can be measured, for example via a technique described in the Examples below.

### V. Kits Containing Agents for Treating Ischemia

In some embodiments of the presently disclosed subject matter, there are provided articles of manufacture and kits containing pharmaceutical compositions for treating ischemia in a subject. In some embodiments, the presently disclosed subject matter provides a kit comprising a pharmaceutical composition for treating ischemia in a subject, the composition comprising an agent adapted to modulate oxidative and/or glycolytic metabolism in ischemic cells in the subject, and a pharmaceutically acceptable excipient; and a delivery device for administering the pharmaceutical composition to a subject. In some embodiments, the delivery device can comprise a syringe. In some embodiment, the agent adapted to modulate oxidative and/or glycolytic metabolism in ischemic cells in the subject comprises one or more gene therapy constructs and compositions produced in accordance with the presently disclosed subject matter, and which can be used, for instance, for therapeutic applications described above.

In some embodiments, the article of manufacture comprises a container with a label. Suitable containers include, for example, bottles, vials, and test tubes. The containers can be formed from a variety of materials such as glass or plastic. The container holds a composition which includes an active agent that is effective for therapeutic applications, such as described above. The active agent in the composition can comprise one or more gene therapy constructs or compositions of the presently disclosed subject matter. The label on the container indicates that the composition is used for a particular therapy or non-therapeutic application, and can also indicate directions for either in vivo, in vitro, or ex vivo use.

In some embodiments, a kit can comprise compositions for use in treating, preventing or ameliorating ischemia and/or ischemic injuries associated therewith and/or diseases or conditions associated with ischemia.

In some embodiments the kit can comprise an gene therapy construct or composition for increasing oxidative metabolism in mitochondria in ischemic cells and/or increasing glycolytic metabolism in ischemic cells in a subject in need thereof. In some embodiments the kit can comprise a gene therapy construct encoding a Cox6a2 polypeptide or a PFKFB3 polypeptide. In some embodiments, the kit can comprise a small molecule pharmacological agent that increases glycolytic metabolism in hypoxic cells. A kit of the presently disclosed subject matter will typically comprise the container described above and one or more other containers comprising materials desirable from a commercial and user standpoint, including buffers, diluents, filters, needles, syringes, and package inserts with instructions for use.

### VI. Methods of Determining Suitable Treatment Course for PAD Patients

In some embodiments, the presently disclosed subject matter provides a method of determining if a PAD patient is unlikely to respond to conventional therapeutic interventions such as surgery, endovascular interventions (e.g., stenting), revascularization techniques (e.g., percutaneous revascularization techniques, such as vascular bypass or angioplasty), and/or physical activity. By unlikely to respond is meant a patient who is more likely than the average PAD patient of having a negative outcome (e.g., limb amputation) despite the conventional therapeutic intervention or who has an at least 25%, 50%, 60%, 70%, 75%, 80%, 85%, 90%, or 95% chance of having a negative outcome (e.g., limb amputation) despite recieving the conventional therapeutic intervention. Thus, in one aspect, the presently disclosed subject matter provides a diagnostic metric for PAD patient at greater risk of tissue loss despite surgical or other conventional limb salvage attempts at restoring blood flow.

In some embodiments, the presently disclosed subject matter provides a method of selecting patient who are most likely to respond well to the therapeutic methods of the presently disclosed subject matter. For example, the patients who are less likely to respond to conventional treatment options aimed at restoring blood flow can, in some embodiments, be selected as being patients likely to receive the most benefit from or be most suitable for the presently disclosed therapeutic methods (e.g., treatment with an agent adapted to modulate oxidative and/or glycolytic metabolism in ischemic cells).

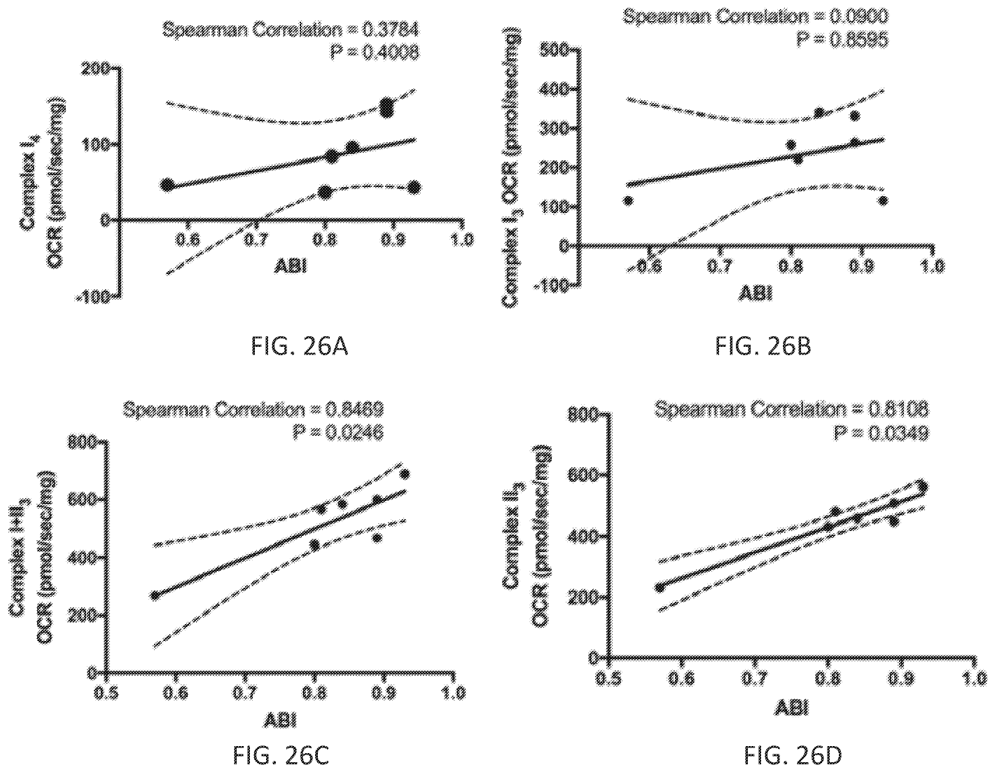

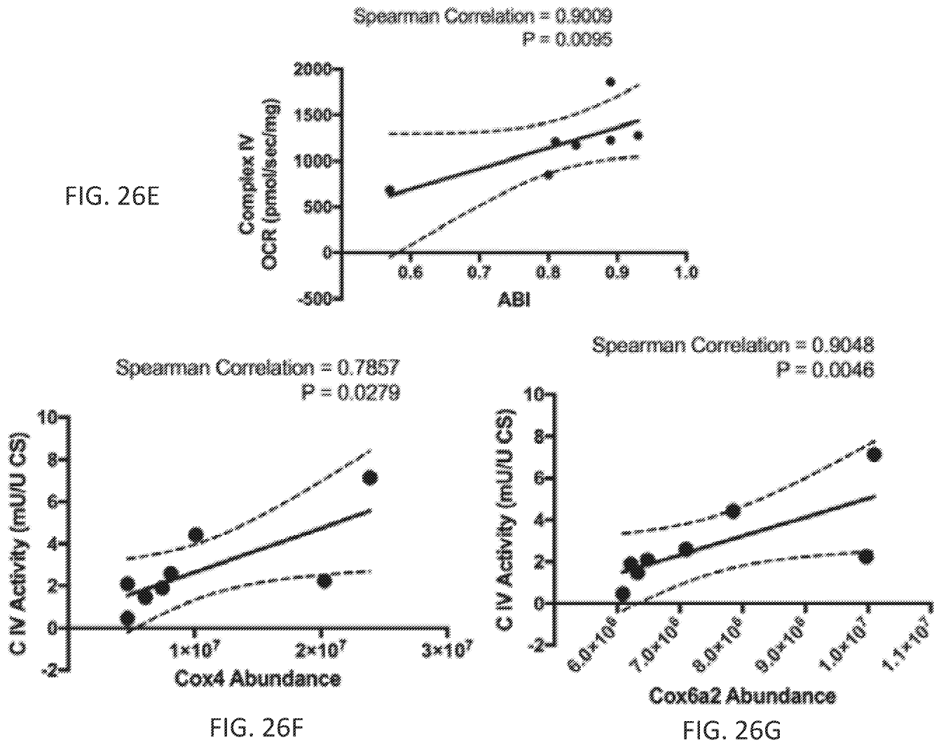

More particularly, as described in Example 3, below, according to one aspect of the presently disclosed subject matter, it has been found that direct correlations can be drawn in claudicating patients between permeabilized myofiber mitochondrial function and Complex I+II₃, Complex II₃, and Complex IV. See FIGS. 26A-26E. Additionally, direct correlations can be drawn between Complex IV activity and the protein abundances of Cox4 and Cox6a2. See FIGS. 26F and 26G. Collectively, these measures demonstrate the diagnostic viability of using permeabilized myofiber function and/or protein abundances of complex IV components to diagnostically differentiate peripheral arterial disease presentation (e.g., between IC and CLI) and identify patients at greater susceptibility for negative events.

Accordingly, in some embodiments, the presently disclosed subject matter provides a method of classifying a subject having PAD (e.g., a patient that has been diagnosed as having PAD) as unlikely to respond to an endovascular therapeutic intervention, a revascularization therapeutic intervention and/or a therapeutic intervention comprising physical activity, the method comprising: providing a sample from a subject having PAD, wherein the sample comprises myofibers from skeletal muscle; measuring mitochondrial function in the sample; and classifying the subject as being unlikely to respond to an endovascular therapeutic intervention, a revascularization therapeutic intervention and/or a therapeutic intervention comprising physical activity based on mitochondrial function. In some embodiments, the subject is classified as as being unlikely to respond to an endovascular therapeutic intervention, a revascularization therapeutic intervention and/or a therapeutic intervention comprising physical activity when the sample has a Complex IV oxygen consumption rate below 2,000 picomoles per second per milligram myofiber, a Complex II₃ oxygen consumption rate of below 1,000 picomoles per second per milligram myofiber, and/or a Complex I+II₃ oxygen consumption rate of below 1,000 picomoles per second per milligram microfiber. In some embodiments, the subject is classified as being unlikely to respond to an endovascular therapeutic intervention, a revascularization therapeutic intervention and/or a therapeutic intervention comprising physical activity when the sample has a Complex IV oxygen consumption rate below about 1,800, about 1,600, about 1,400, about 1,200, about 1,000, about 900, about 800, about 700, or about 600 or less pmol/sec/mg myofiber. In some embodiments, the subject is classified as being unlikely to respond to an endovascular therapeutic treatment, a revascularization therapeutic intervention, and/or a therapeutic intervention comprising physical activity when the sample has a Complex II₃ and/or a Complex I+II₃ oxygen consumption rate of below about 900, 800, 700, 600, or 500 pmol/sec/mg myofiber. In some embodiments, the method further comprises administering to the subject classified as unlikely to respond to the endovascular therapeutic intervention, the revascularization therapeutic intervention and/or a therapeutic intervention comprising physical activity an agent adapted to modulate oxidative and/or glycolytic metabolism in ischemic cells in the subject. In some embodiments, the agent is adapted to provide Cox6a2 and/or PFKFB3 to the ischemic cells in the subject.

Mitochondrial function in the sample can be measured by any suitable technique, including those described below in the Examples and/or using commercially available kits. For example, mitonchondrial function can be assessed based on correlations drawn between Complex IV activity and the protein abundances of Cox4 and Cox6a2, with protein abudances further assessed based on comparsion to a control subject, such as a healthy subject of the same species and age. In some embodiments, the sample is a skeletal muscle biopsy sample from the subject.

### EXAMPLES

The following Examples provide illustrative embodiments. In light of the present disclosure and the general level of skill in the art, those of ordinary skill will appreciate that the following Examples are intended to be exemplary only and that numerous changes, modifications, and alterations can be employed without departing from the scope of the presently claimed subject matter.

### General Methods and Materials for Examples 1-3

Tissue Acquisition: Twenty-six healthy adults without PAD (HA), twenty PAD patients with intermittent claudication (IC), and nineteen patients with critical limb ischemia (CLI) were recruited through print advertising (HA) or identified by vascular surgeons at East Carolina University Brody Medical Center and the University of Nebraska Medical Center (UNMC) and Veterans Affairs Nebraska-Western Iowa. Inclusion criteria comprised of patients representative of all clinical classifications of PAD. Exclusion criteria consisted only of CLI amputation patients who previously provided biological specimens from the contralateral limb. All data collection was carried out by blinded investigators.

Percutaneous muscle biopsy samples were taken from the lateral gastrocnemius muscle of age-matched volunteer HA and identified IC patients. The skin of the muscle biopsy sight was first cleansed with a povidone-iodine swab and then anesthetized with 5 cc of lidocaine. A small incision was made, and the muscle biopsy sample was aspirated through a 5 mm Bergstrom needle. Tissue obtained from CLI patients occurred inside the operating room immediately following limb amputation which was performed without the use of a tourniquet. Muscle specimens from CLI patients were collected from the same anatomical location of the gastrocnemius muscle as the percutaneous muscle biopsies performed in HA and IC patients. Part of the muscle biopsy sample was immediately flash frozen in liquid nitrogen and stored at −80° C. until subsequent analysis. Other portions of the muscle biopsy (˜80-150 mg) were used for human primary muscle cell isolation and permeabilized myofiber mitochondrial experiments.

Transcriptome Sequencing: Whole genome (RNA) sequencing was performed to identify differentially expressed genes between patient groups. Total RNA was extracted using Qiagen RNeasy Midi kits (Qiagen, Hilden, Germany). RNA sequencing was performed by Quick Biology Inc. (Pasadena, Calif., United States of America). RNA integrity was checked by Agilent Bioanalyzer 2100 (Agilent Technologies, Santa Clara, Calif., United States of America); only samples with clean rRNA peaks were used. Library for RNA-Seq was prepared according to KAPA Stranded mRNA-Seq poly(A) selected kit with 201-300 base pair (bp) insert size (KAPA Biosystems, Wilmington, Mass., United States of America) using 250 nanograms (ng) total RNA as input. Final library quality and quantity was analyzed by Agilent Bioanalyzer 2100 (Agilent Technologies, Santa Clara, Calif., United States of America) and Life Technologies Qubit3.0 Fluorometer (Thermo Fisher Scientific, Waltham, Mass., United States of America). 150 bp paired end reads were sequenced on Illumina HighSeq 4000 (Illumnia Inc., San Diego, Calif., United States of America). The reads were first mapped to the latest UCSC transcript set using Bowtie2 version 2.1.0 (see Langmead and Salzberg, Nat Methods, 2012, 9(4):357-359) and the gene expression level was estimated using RSEM v1.2.15. See Li and Dewey, BMC Bioinformatics, 2011, 12:323. TMM (trimmed mean of M-values) was used to normalize the gene expression. Differentially expressed genes were identified using the edgeR program. See Robinson et al., Bioinformatics, 2010, 26(1):139-130. Genes showing altered expression with p<0.05 and more than 1.5-fold changes were considered differentially expressed. Goseq was used to perform the GO enrichment analysis and Kobas was used to perform the pathway analysis. Heatmaps were generated with the Pheatmap program using a log₂ [Fold Change from HA]. Validation of sequencing results was performed using quantitative real time PCR of selected genes. Gene ontology enrichment analysis was performed to identify target pathways/cellular processes/biological functions.

Skeletal Muscle Mitochondria: Skeletal muscle mitochondrial function was assessed by respirometry with permeabilized myofibers prepared from fresh muscle biopsy samples. See Ryan et al., J Physiol. 2014, 592(Pt15):3231-3241. Mitochondrial content was determined by citrate synthase activity and cardiolipin content measured by mass spectrometry. See Sullivan et al., J. Biol. Chem., 2018, 293(2):466-483. Specific activities of mitochondrial oxidative phosphorylation (OXPHOS) complexes were measured spectrophotometrically. See Shaikh et al., Biochemistry, 2014, 53(35):5589-5591. Protein content of OXPHOS complexes was determined by Western blotting of skeletal muscle lysates.

Muscle Progenitor Cells (MPCs): Primary human muscle precursor cells (human myoblasts) derived from fresh muscle biopsy samples were obtained from patients. MPCs were differentiated into myotubes by serum withdrawal. MPC respiration was assessed using a Seahorse XF24 analyzer (Agilent Technologies, Santa Clara, Calif., United States of America). MPC mitochondrial content and volume was assessed by citrate synthase activity and immunofluorescent confocal imaging. MPC human mitochondrial gene expression levels was determined by a gene expression array (Qiagen Cat #PAHS-008ZA, Qiagen, Hilden, Germany) using qRT-PCR.

Statistical Analysis: Data are presented as mean±SEM unless otherwise specified. Comparisons of data with more than 2 groups were performed using ANOVA with Tukey's post-hoc for multiple comparisons. In all cases, P<0.05 was considered statistically significant.

### Example 1

### Mitochondriopathy in CLI Patients

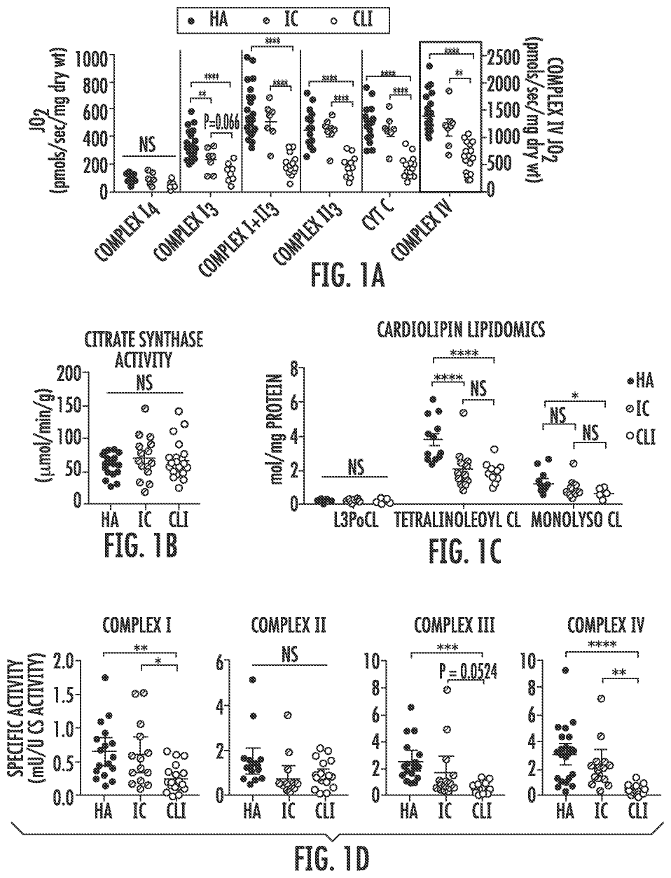

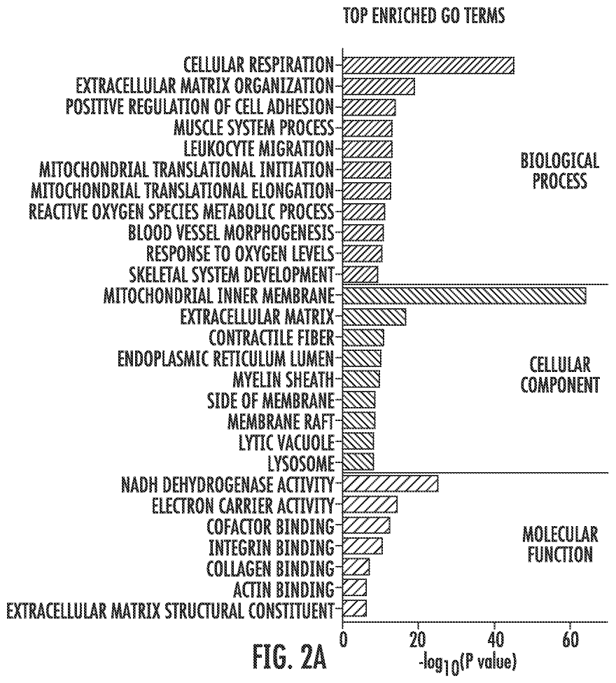

Tissue samples were uniformly collected from the gastrocnemius, 10 cm distal to the tibial tuberosity or from proximal regions free of necrotic lesions or non-healing wounds in healthy adult (HA), claudicating (IC) or critical limb ischemia (CLI) amputees. Histological assessments confirmed the absence of necrosis in the biopsy specimens, although IC and CLI specimens displayed morphological indicators of myopathy (smaller and non-uniform myofiber sizes, myofibers with central nuclei). Functional mitochondrial phenotyping was performed with fresh permeabilized myofiber bundles, which facilitates the analysis of mitochondrial function in the native biological state of the organelle in muscle fibers. CLI patients exhibited severe impairments in mitochondrial oxygen consumption compared with both HA and IC groups. See FIG. 1A. In contrast, IC patients only displayed a statistically significant impairment in Complex I (NADH dehydrogenase) supported ADP-stimulated respiration. Mitochondrial content/density differences were excluded as the mechanisms for this difference via parallel analysis of citrate synthase activity and cardiolipin content (a lipid species present only in mitochondrial membranes). See FIGS. 1B and 1C. Complex-specific enzyme activity assays for Complexes I, II, III, and IV of the electron transport system (NADH dehydrogenase, succinate dehydrogenase, ubiquinone-cytochrome c oxidoreductase, and cytochrome c oxidase) in muscle homogenates confirmed a unique mitochondrial dysfunction in CLI patients. These assays indicated impairments in Complexes I, III, and IV in CLI patient. See FIG. 1D.

### Example 2

### Bioenergetic Gene Expression Signature of CLI Patients

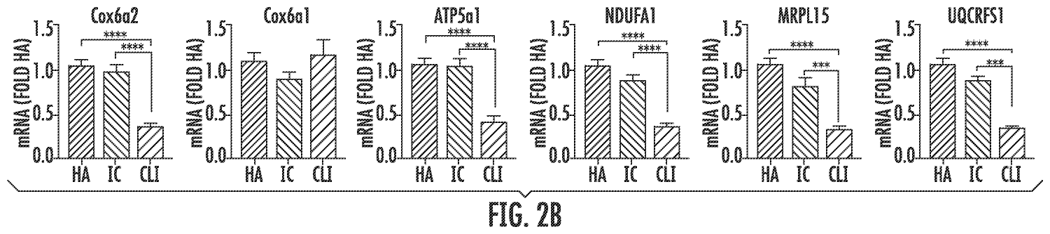

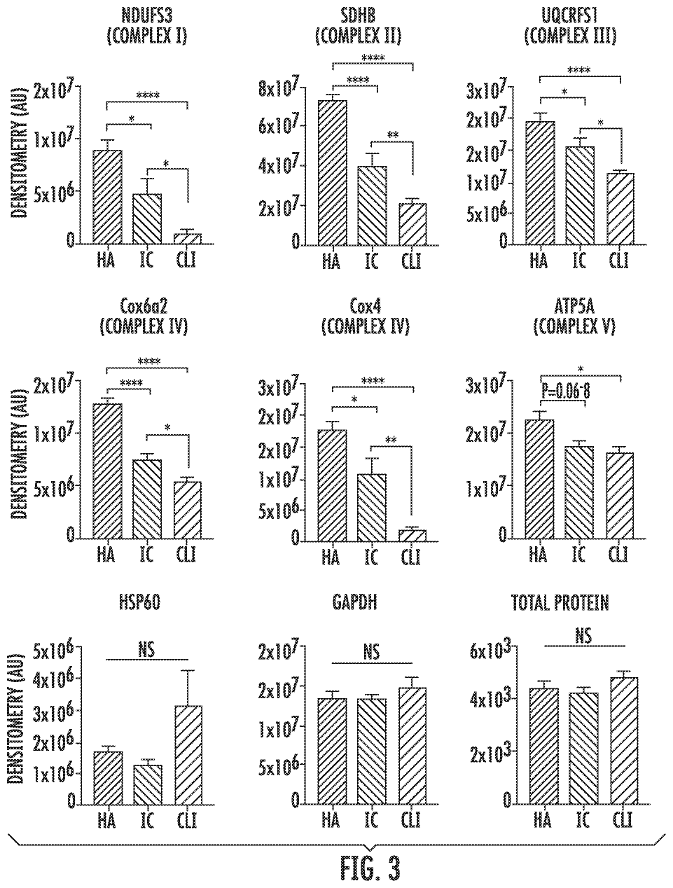

Total RNA was isolated from patient muscle samples and whole genome (mRNA) sequencing was performed to examine gene expression profiles in these patients. RNA sequencing and gene ontology (GO) enrichment analysis revealed a unique defect in the transcriptional program of mitochondrial metabolism only in CLI patients. GO analysis revealed the most enriched pathways as cellular respiration, mitochondrial inner membrane, and NADH dehydrogenase activity. See FIG. 2A. A heatmap of RNA expression for the genes involved in cellular respiration highlighted the uniform and striking differences observed in CLI patients. Confirmatory assessments were performed using qRT-PCR for several identified targets. See FIG. 2B. Western blotting analysis verified that the observed transcriptional deficits translated to reductions in protein abundances for selected mitochondrial proteins. Interestingly, lower expression of several proteins involved in oxidative phosphorylation (NDUFS3, SDHB, UQCRFS1, Cox4, Cox6a2, and ATP5a) was observed in both IC and CLI compared with the HA. See FIG. 3. The magnitude of reduction in CLI patient tissues was consistently greater than that of IC patients for all targets except ATP5a. Collectively, these findings demonstrate a pervasive attenuation of mitochondrial protein expression in CLI patients. IC patients, however, are able to maintain a sufficient transcriptional program that better support tissue mitochondrial respiration and physical activity.

### Example 3

### Mitochondrial Function and Bioenergetic Gene Expression Impairments are Retained in Muscle Progenitor Cells from CLI Patients

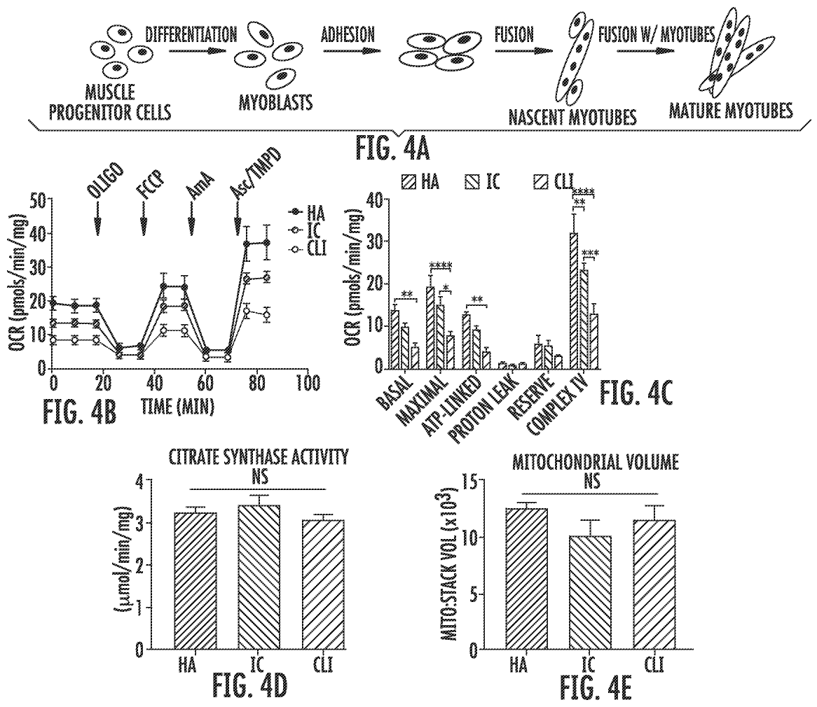

Recent work identified an acute increase in progenitor cell numbers in exercising claudicant tissues. See McDermott et al., JAMA, 2017, 38(21):2089-2098. To investigate whether the impaired mitochondrial transcriptional defect observed in muscle tissue could have a lasting impact on the health and function of MPCs, primary MPCs were isolated from patient gastrocnemius specimens and differentiated into myotubes in standard normoxic culture conditions. See FIG. 4A. CLI patient cells possessed decreased maximal and Complex IV-linked oxygen consumption compared to both HA and IC. See FIGS. 4B and 4C. Citrate synthase activity (see FIG. 4D) and confocal imaging of mitochondrial volume and morphology (GFP-conjugated TOMM20) (see FIG. 4E) confirmed similar mitochondrial content in all cells. A qRT-PCR gene array focused on mitochondrial metabolism revealed that the gene expression defects observed in CLI patient tissues are retained in CLI patient MPCs under norm oxic and nutrient rich conditions.

In summary, the present data show that CLI patients suffer from a previously unknown mitochondriopathy that extends beyond muscle myofibers to negatively impact muscle progenitor cell function. A unique gene expression signature uniformly occurred in CLI patients, while claudicating PAD patients had normal mitochondrial gene expression and function in both myofibers and MPCs. Reduced mitochondrial function in the myofibers is a pathophysiologic response to limb ischemia that is liable to be an “all or none” scenario. In this context, mitochondria likely hold on for as long as possible (with all gene expression maintained) until a catastrophic failure to adequately support cellular functions around them occurs. Mitochondriopathies have also been implicated in disease etiology for cardiovascular diseases. The presently disclosed studies identify an important biological outcome distinguishing IC and CLI patient tissues and reveal a potential diagnostic metric for PAD patients at greater risk of tissue loss despite surgical limb salvage attempts at restoring blood flow. Direct correlations can be drawn in claudicating patients between permeabilized myofiber mitochondrial function and Complex I+II₃, Complex II₃, and Complex IV. See FIGS. 26A-26E. Additionally, direct correlations can be drawn between Complex IV activity and the protein abundances of Cox4 and Cox6a2. See FIGS. 26F and 26G. Collectively, these measures demonstrate the diagnostic viability of using permeabilized myofiber function and/or protein abundances of complex IV components to diagnostically differentiate peripheral arterial disease presentation and potentially identify patients at greater susceptibility for negative events. Given the known negative independent influences of risk-factors on limb muscle mitochondria, these studies also indicate that limb muscle mitochondrial bioenergetics can potentially serve as a site of convergence in the pathophysiologies of risk factors such as diabetes and CLI.

### General Methods and Materials for Examples 4-10

Animals: Experiments were conducted on adult (12 weeks) C57BL/6J (BL6; N=74), BALB/c (N=162), 129s1/SvImJ (N=8), A/J (N=8), and C3H/HeJ (N=8) male mice purchased from Jackson Laboratories (Bar Harbor, Me., United States of America). First generation offspring were generated by crossing C57BL/6J and BALB/c mice (N=28). Additional female C57BL/6J (BL6; N=36) and BALB/c (N=36) mice aged 4 weeks old were used for cell isolations. All rodents were housed in a temperature (22° C.) and light controlled (12-hour light/12-hour dark) room and maintained on standard chow with free access to food and water. Acute hindlimb ischemia (HLI) (see Dokun et al., Circulation, 2008, 117:1207-15) was performed by anesthetizing mice with intraperitoneal injection of ketamine (90 mg/kg) and xylazine (10 mg/kg) and surgically inducing unilateral hindlimb ischemia by ligation and transection of the femoral artery from its origin just below the inguinal ligament.

Limb muscle function, morphology and regeneration: Isometric force measurements were performed using EDL muscles as previously described See Ryan et al., Diabetes, 2016, 65:2553-68; and Spangenburg et al., J. Physiol., 2008, 586:283-91. Skeletal muscle morphology and vessel density were assessed by standard light microscopy and IF microscopy using 10-μm-thick transverse sections from the tibialis anterior (TA) muscle as previously described. See McClung et al., Circulation, 2017, 136:281-296; and Ryan et al., Diabetes, 2016, 65:2553-68. Transmission electron microscopy was performed on perfusion fixed mice at day seven post-HLI.

Mitochondrial isolation and functional assays: Skeletal muscle mitochondria were isolated from the plantar flexors (i.e. gastrocnemius, soleus, and plantaris) muscles of both control and ischemic limbs as previously described. See Ryan et al., Diabetes, 2016, 65:2553-68. High-resolution O₂ consumption measurements were performed to assess respiratory function as previously described. See Ryan et al., J Mol Cell Cardiol. 2016, 97:191-196; and Ryan et al., Diabetes, 2016, 65:2553-68. Specific activities of mitochondrial ETS enzymes and citrate synthase activity were measured spectrophotometrically. See Shaikh et al., Biochemistry, 2014, 53:5589-91.

Primary Cell Isolation and Culture: Primary murine muscle precursor cells (mouse myoblasts) derived from peripheral hindlimb muscles were prepared as previously described. See McClung et al., Circulation, 2017, 136:281-296; and McClung et al., The American journal of pathology, 2012, 180:2156-69. Primary fibroblasts were obtained through a pre-plating process on non-coated plastic culture dishes. ECs were isolated as previously described (see Fehrenbach et al., American journal of physiology lung cellular and molecular physiology, 2009, 296:L1096-103) with slight modifications. Cellular respiration was assessed in vitro using a Seahorse XF24 analyzer (Agilent Technologies, Santa Clara, Calif., United States of America) as previously described. See Ryan et al., J Mol Cell Cardiol. 2016, 97:191-196. Muscle cell proliferation and differentiation were assessed as previously described. See McClung et al., Circulation, 2017, 136:281-296.

Human participants: Sixteen healthy adult (6 males, 10 females, Age=57.4±1.4 years) and nine patients with critical limb ischemia (CLI) (6 males, 3 females, Age=62.4±3.4 years) were enrolled for muscle biopsy collection. Tissue obtained from CLI patients occurred inside the operating room immediately following limb amputation.

Skeletal muscle biopsies: Percutaneous muscle biopsy samples (see Ryan et al., J Physiol. 2014, 592:3231-41) were taken from the lateral gastrocnemius muscle of age-matched healthy adults. Part of the muscle biopsy sample was immediately flash frozen in liquid nitrogen and stored at −80° C. until subsequent analysis. Other portions of the muscle biopsy (˜80-150 mg) were used for human primary muscle cell isolation.

Virus Generation: The full viral cassette was cloned via In-Fusion (Takara Bio Inc., Kusatsu, Japan) into the pAdenoX-PRLS vector (Takara Bio Inc., Kusatsu, Japan). Adenoviruses were generated by transfection of Adeno-X 293 cells with PacI linearized pAdenoX vectors using calcium phosphate transfection method. Adenoviral infections of cells were performed at MOI=200 in the presence of 3 μg/ml polybrene. Adeno-associated viruses (AAV2 ITR vector from Dr. Samulski at the University of North Carolina at Chapel Hill) for GFP and Cox6a2 were generated and purified by column chromatography at either the University of North Carolina Viral Vector Core Facility (Chapel Hill, N.C., United States of America) or Vector Biolabs (Malvern, Pa., United States of America). AAVs (serotype 9) were locally delivered via intramuscular injections of the hindlimb musculature (plantarflexors and dorsiflexors) at 5×10¹⁰ vg/injection site two weeks prior to HLI.

Statistics: Data are presented as mean ±SEM. Comparisons between 2 groups were performed by Student's two-tailed t-test. Comparisons of data with more than 2 groups were performed using ANOVA with Tukey's post-hoc multiple comparisons. Repeated-measures ANOVA followed by Tukey's post-hoc were performed when appropriate. Nonparametric Mann-Whitney testing was used to determine differences between the distributions of necrosis scores between groups. In all cases, P<0.05 was considered statistically significant.

### Example 4

### Ischemic Mitochondriopathy Segregates Across Inbred Mouse Strains

In pre-clinical models of PAD, inbred mouse strains display remarkable variation in their response to comparable amounts of ischemia. Strains such as C57BL/6J and 129s1/SvImJ display remarkable protection from ischemia, while BALBc/J, NJ, and C3H/HeJ suffer poor perfusion recovery and larger infarct sizes with ischemia. Accordingly, studies were preformed to investigate whether mitochondrial function displayed a divergent phenotype between favorable (C57BL/6J, 129s1/SvImJ, and F1_{[BL6xBALB/c]}) and poor (BALB/c, A/J, and C3H/HeJ) responding inbred mouse strains following HLI.

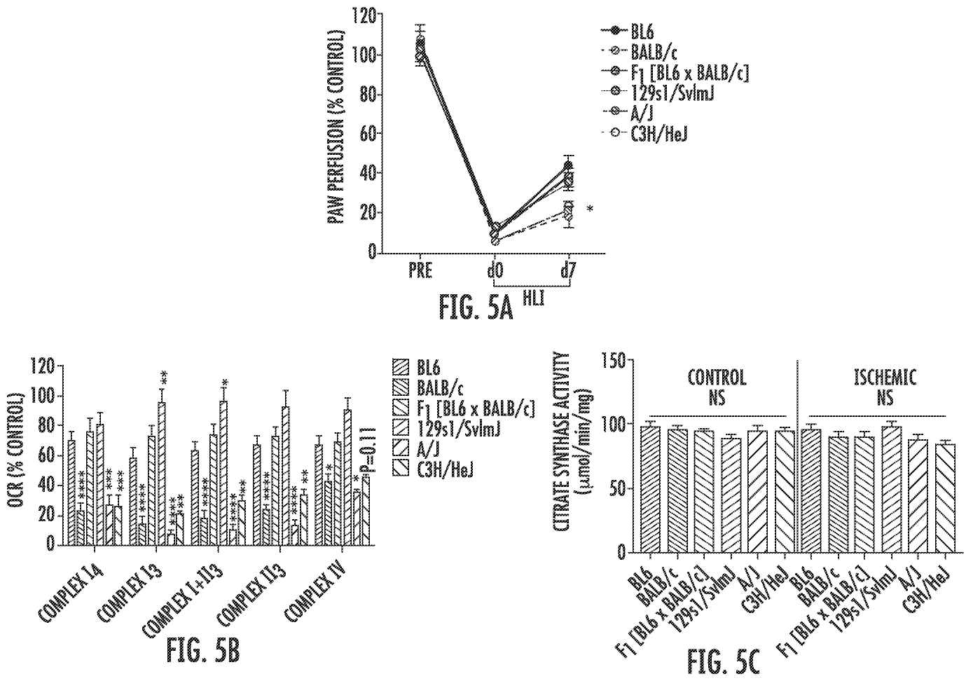

In addition to greater perfusion recovery (see FIG. 5A), C57BL/6J, 129s1/SvImJ, and F1_{[BL6xBALB/c]} mice demonstrated significantly greater rates of oxygen consumption in ischemic limb muscle mitochondria compared with BALB/c, A/J, and C3H/HeJ mice. See FIG. 5B. Further, these respiratory deficits occurred in the absence of reductions in mitochondrial content, as assessed by citrate synthase activity. See FIG. 5C.

### Example 5

### Rapid-Onset and Chronic Skeletal Muscle Mitochondrial Dysfunction in Ischemic BALB/c Mice

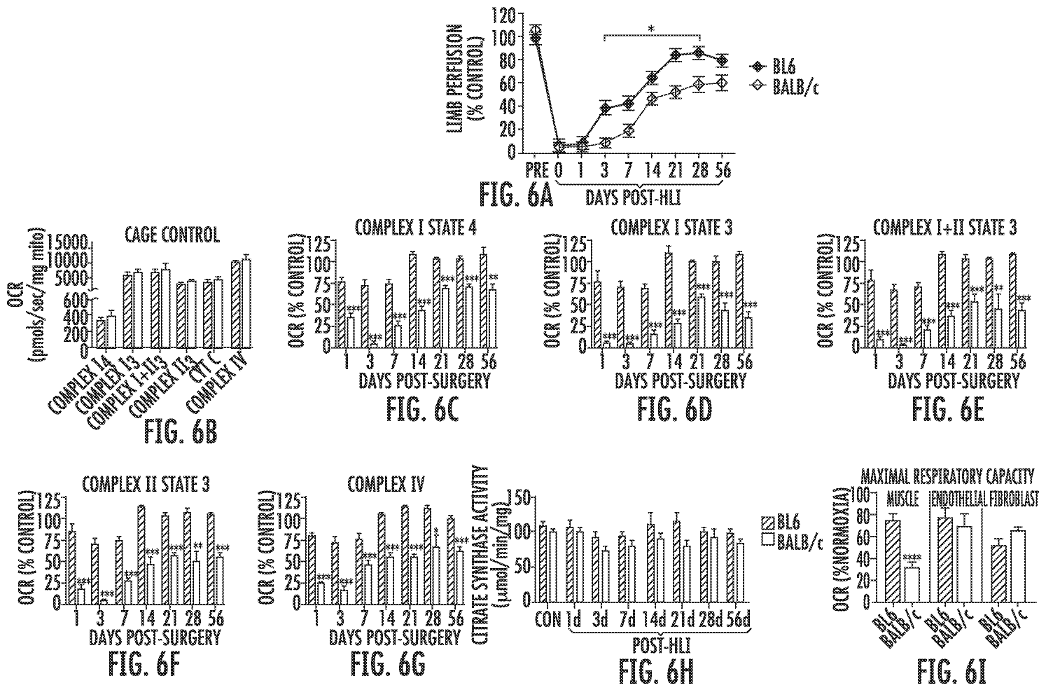

In view of the parallel outcomes between limb muscle mitochondrial function and limb blood flow across ischemic inbred strains described in Example 4, the chronology of mitochondrial function following HLI was further studied in C57BL/6J (BL6) and BALB/c mice. Consistent with previous reports, BALB/c, mice displayed reduced limb perfusion recovery compared to BL6 following HLI. See FIG. 6A. High-resolution respirometry was first performed in cage control limb muscle mitochondria using a variety of substrate/inhibitor combinations (designed to assess multiple sites in the mitochondrial electron transport system (ETS) to verify similar mitochondrial function at baseline. See FIG. 6B. Following HLI, only BALB/c limb muscle mitochondria rapidly (within 24 hours post-HLI) lost respiratory function under all substrate conditions (see FIGS. 6C-6G and 6I), suggesting complete disruption of the ETS prior to significant differences in limb blood flow in this strain which begin at day-3 post-HLI. See FIG. 6A. The observed deficit was sustained for 8-weeks post-HLI in all substrate conditions in the ischemic BALB/c limb muscles. It also occurred in the absence of overt changes in mitochondrial content. See FIG. 6H.

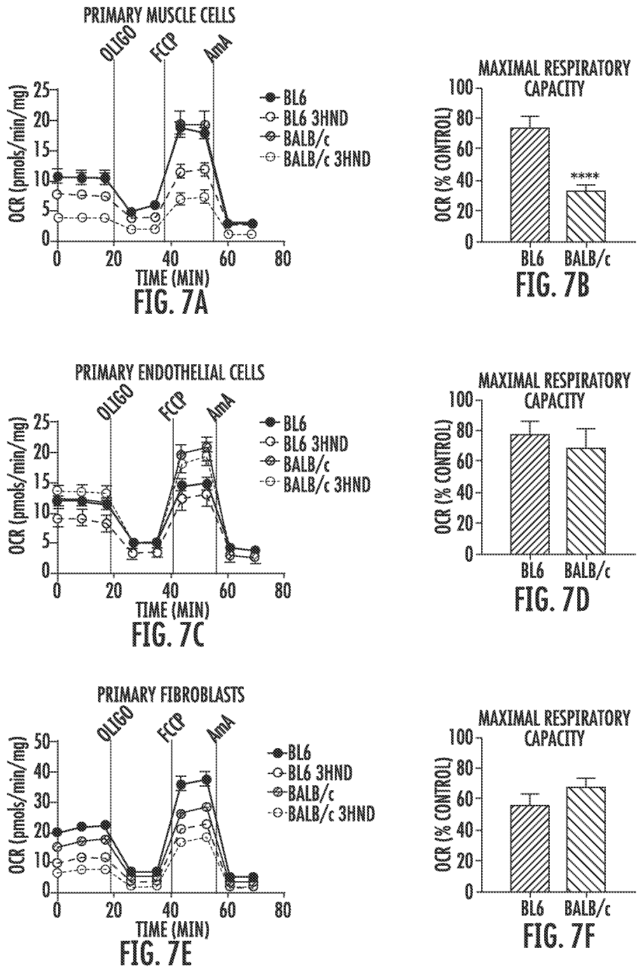

To establish cell specificity of ischemic mitochondrial dysfunction, primary limb muscle, fibroblast, and endothelial cells were isolated from BL6 and BALB/c mice and subjected to hypoxia and nutrient deprivation (HND) in vitro. See FIGS. 7A-7F. Similar to observations in cage control mice, maximal respiratory capacity was similar between BL6 and BALB/c muscle progenitor cells under normoxia. When exposed to 3 hours of HND, BALB/c MPCs, but not endothelial cells or fibroblasts, display a significant reduction in respiratory capacity.

### Example 6

### Chronic Skeletal Muscle Myopathy in BALB/c Mice Parallels Mitochondriopathy

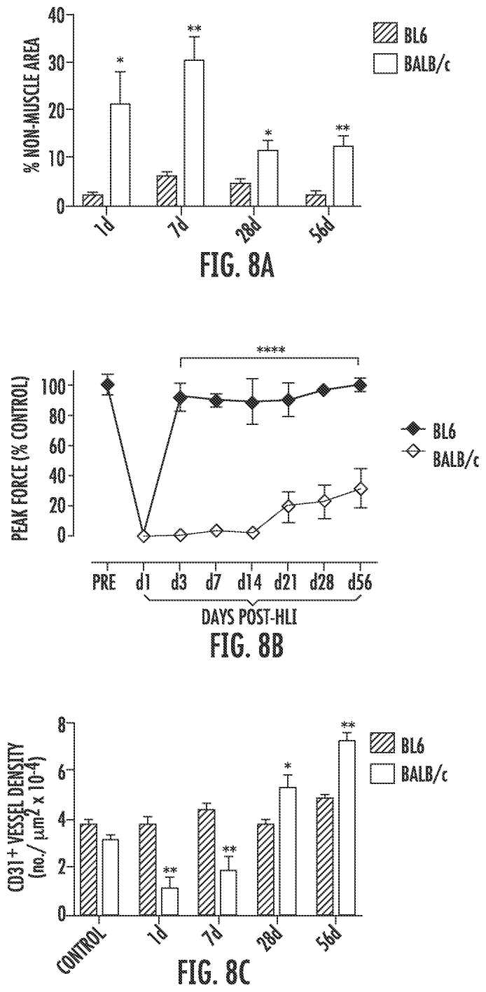

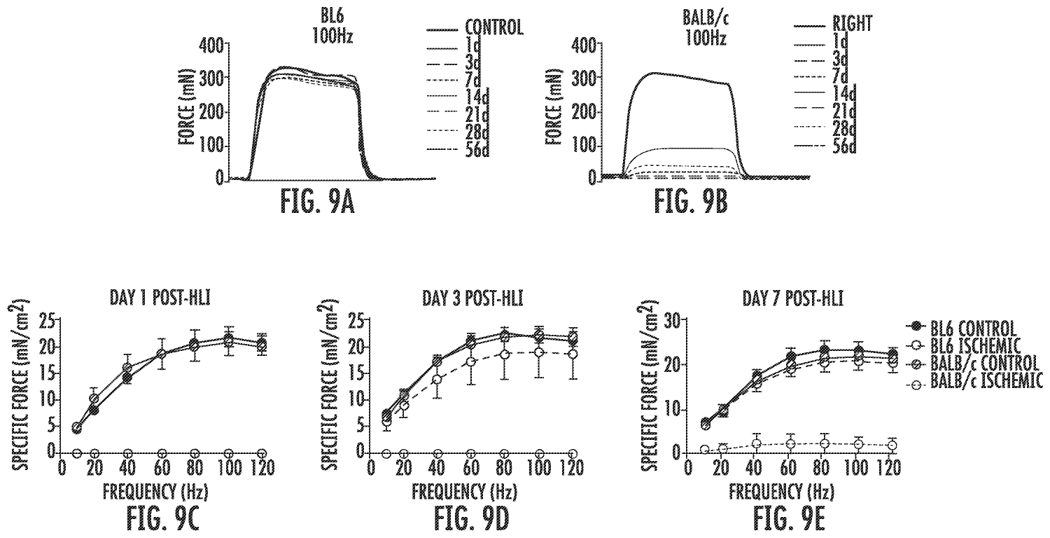

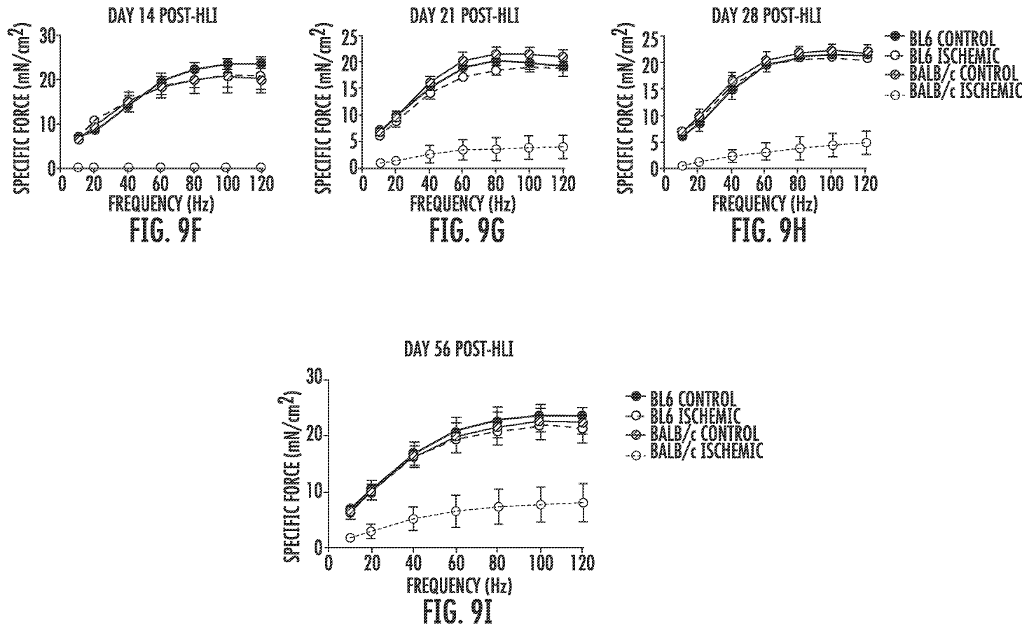

Histological analysis of the ischemic limb skeletal muscle demonstrated the rapid onset of myopathy (non-myofiber area; see FIG. 8A) and functional deficits (isometric muscle force; see FIG. 8B and FIGS. 9A-9I) that persist out to 8-weeks post HLI in BALB/c, but not BL6, mice. Electron micrographs at day 7 post-HLI revealed that BALB/c mice exhibit a complete disruption of myofilament ultrastructure and mitochondrial morphology. BALB/c limb muscle also suffered a rapid (1 day post HLI) reduction in capillary density (CD31⁺ vessels), but recovered by 28 days post HLI (compared to control muscle). See FIG. 8C. Capillary density remained steady in BL6 ischemic limb muscle across the same time-span.

### Example 7

### Cox6a2 Expression Patterns in Inbred Strains of Mice

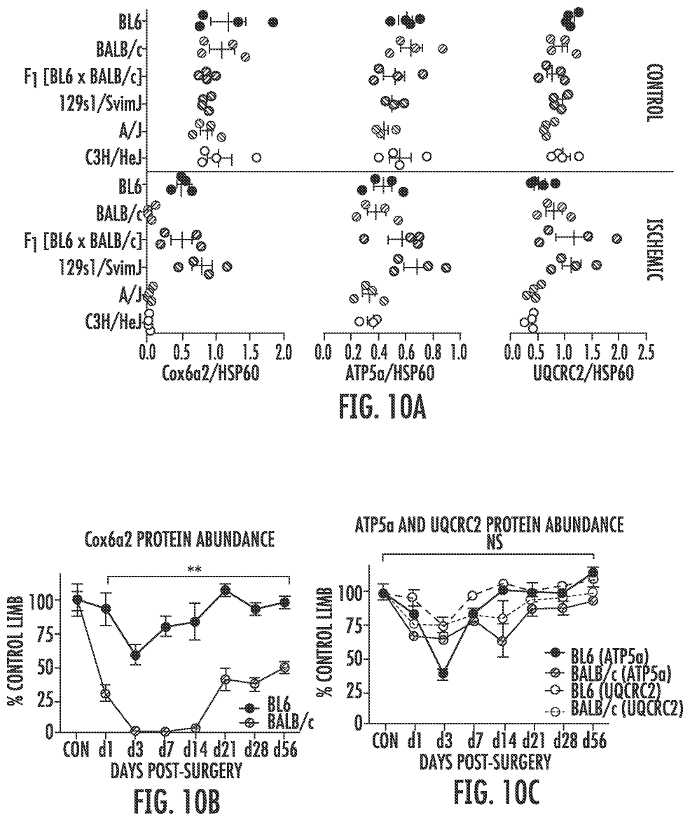

Cox6a2 was the sole known mitochondrial related gene originally identified in Lsq-1. No coding variants were identified after sequencing BL6 and BALB/c mouse Cox6a2. Cox6a2 expression in the ischemic limb muscles across inbred mice at day seven post-HLI was examined via western blotting. Cox6a2 protein abundance was similar across strains at baseline, and was very low (or undetectable) in all strains that displayed perfusion recovery deficits and reduced mitochondrial respiration. See FIG. 10A. Strain differences in expression were not observed in other nuclear-encoded mitochondrial ETS (ATP5a and UQCRC2) or matrix (HSP60—a mitochondrial loading control) proteins. Cox6a2 protein abundance was also examined across the HLI timeline in BL6 and BALB/c mice. Cox6a2 protein rapidly (1 day post HLI) decreases in BALB/c mice and remained decreased for at least 56 days. See FIG. 10B. Strain-dependent differences in protein expression in other nuclear-encoded mitochondrial ETS proteins involved in oxidative phosphorylation (ATP5a and UQCRC2) were not observed across the same time-course. See FIG. 10C.

### Example 8

### Cox6a2 Delivery Rescues Limb Blood Flow and Mitochondrial Respiratory Function in the Ischemic Limb Muscle of BALB/c Mice

Given the severity of mitochondrial dysfunction in BALB/c mice with HLI and the parallel reductions in Cox6a2, the ability of AAV9-mediated re-expression of Cox6a2 to rescue mitochondrial function and improve tissue outcomes was examined. First, by electroporating a green fluorescent protein (GFP)-fused Cox6a2 plasmid into the flexor digitorum brevis (FDB) and visualizing the intact isolated single muscle cells using confocal imaging alongside a fluorescent mitotracker dye, it was confirmed that exogenous Cox6a2 protein product localized within the mitochondria. More particularly in this regard, muscle from a BALB/c mouse was electroplated with a plasmid encoding a Cox6a2-GFP construct. Single myofibers were digested and isolated seven days after electroporation and co-localization was examined via confocal microscopy. Images were captured at 30 times magnification, ensuring Nyquist spatial sampling was appropriate and then co-localization was assessed using ImageJ Software (NIH, Bethesda, Md., United States of America) according to previously described methods.

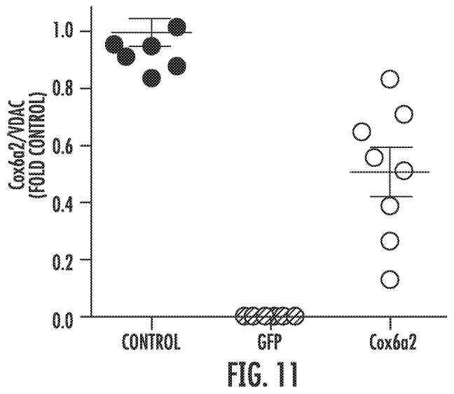

Next, adeno-associated virus serotype 9 (AAV9)-Cox6a2 or AAV9-GFP (control virus group) was delivered locally via intramuscular injections and a heterogeneous re-expression of Cox6a2 protein was verified in ischemic BALB/c limb muscle via western blotting. Quantification of Cox6a2 protein expression as compared to voltage-dependent anion channel (VDAC) protein is shown in FIG. 11.

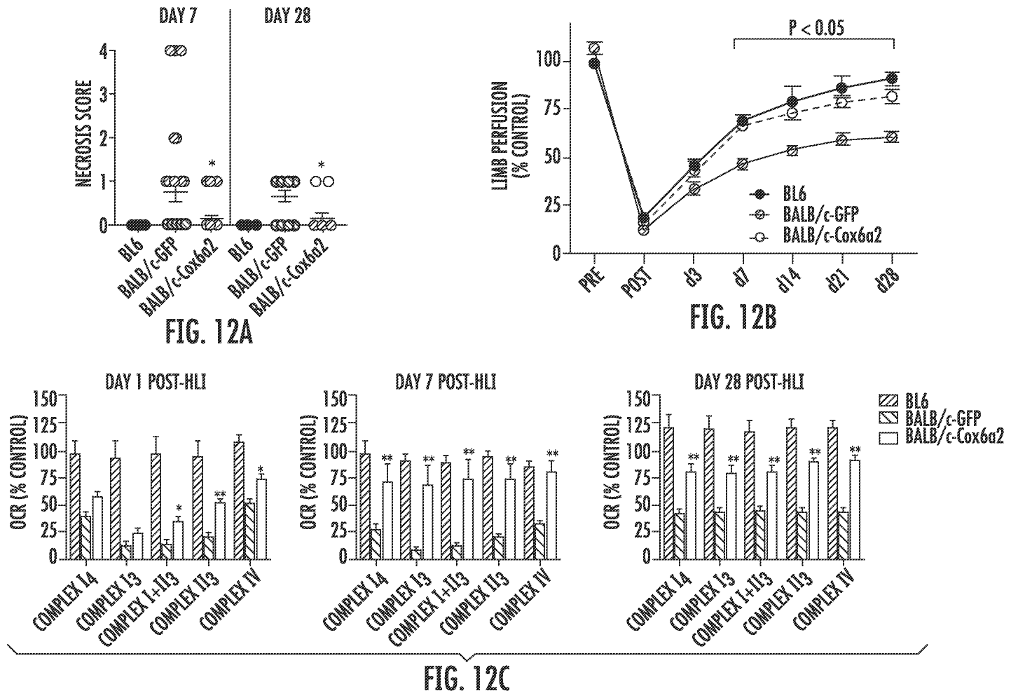

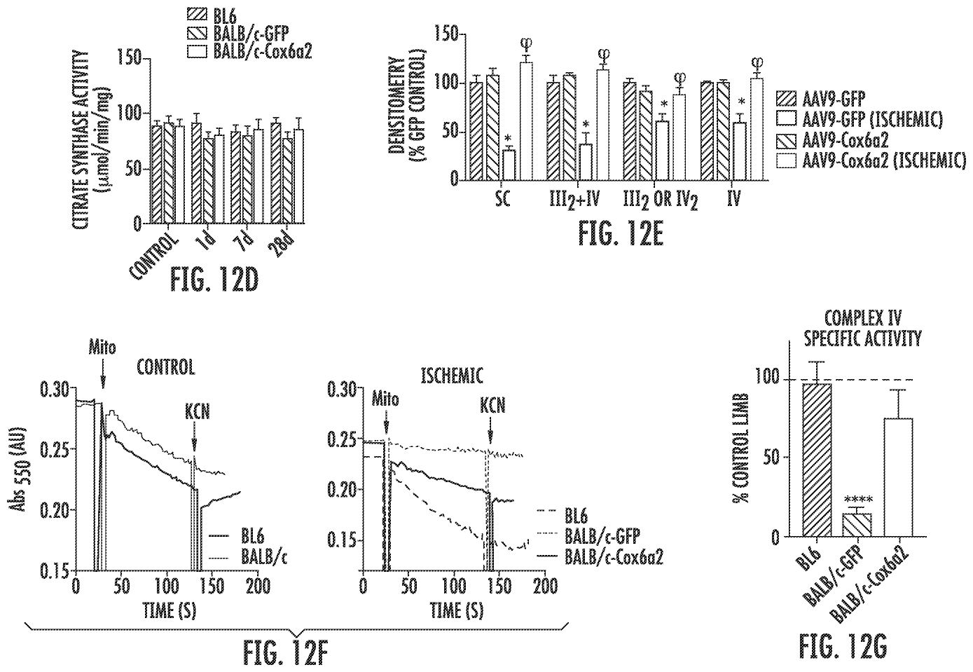

BL6 and BALB/c mice were injected intramuscularly with AAVs encoding GFP (N=64) or Cox6a2 (N=64) and subjected to unilateral HLI. Limb necrosis scores were calculated at day 7 and day 28 post HLI. AAV9-Cox6a2 rescued superficial limb tissue necrosis at both 7days and 28days post HLI (see FIG. 12A) and significantly improved limb blood flow across 28 days post HLI, as determined via quantification of laser doppler perfusion imaging (LDPI) images of the mice. See FIG. 12B. Mitochondrial function was assessed using high-resolution respirometry in mitochondrial isolated from the plantarflexor muscles. See FIG. 12C. As indicated in FIG. 12C, AAV9-Cox6a2 treatment rescued limb muscle mitochondrial respiratory function under various substrate conditions early after ischemia onset (day 1 post HLI) and across all substrate conditions with sustained (day 7 and day 28 post-HLI) ischemia. Changes in mitochondrial respiratory function occurred despite similar mitochondrial content, assessed by citrate synthase activity. See FIG. 12D. To determine whether the restoration of limb muscle mitochondrial function during ischemia can be associated with altered mitochondria supercomplex formation/stability, BN-PAGE was performed to visualize mitochondrial supercomplexes. Mitochondria isolated from the ischemic limb of AAV9-Cox6a2 treated BALB/c mice displayed a greater abundance of mitochondrial supercomplexes and native Complex IV compared with their AAV9-GFP treated counterparts. See FIG. 12E. A specific activity assay of cytochrome c oxidase (Complex (IV) of the mitochondrial electron transport system (ETS) was performed in isolated mitochondria from control and ischemic limbs at day seven post HLI. This further examination of the enzymatic function of cytochrome c oxidase in isolated limb tissue mitochondria demonstrated that AAV9-Cox6a2 delivery significantly improved the specific activity of Complex IV. See FIGS. 12F and 12G.

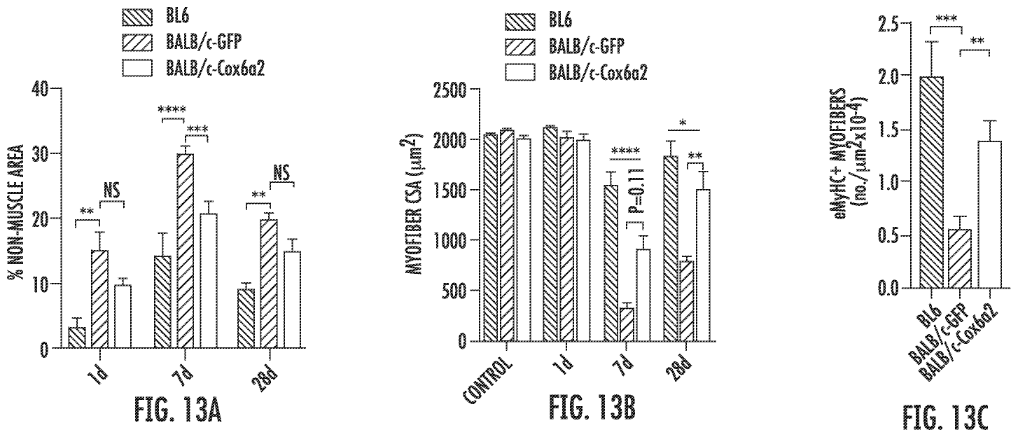

Tibialis anterior (TA) muscle from the mice were stained with hematoxylin and eosin and imaged. From these images, average non-myofiber area and average myofiber cross sectional area were quantified. In addition, TA muscle was stained with 4′,6′-diamidino-2-phenylindole (DAPI) for nuclei and for embryonic myosin heavy chain (eMyHC). AAV9-Cox6a2 rescued ischemic limb muscle fascicular arrangements and myofiber cross-sectional area compared to GFP control (see FIGS. 13A and 13B) and increased regenerating myofiber number (embryonic myosin heavy chain) comparable to values observed in ischemic BL6 muscles at 7days post-HLI. See FIG. 13C. It is believed that these in vivo data provide the first evidence that a skeletal muscle-specific, mitochondrial gene therapy can improve PAD pathology in mice.

### Example 9

### Cox6a2 Expression Regulates Mitochondrial Respiratory Function and Myogenesis in Primary Muscle Cells

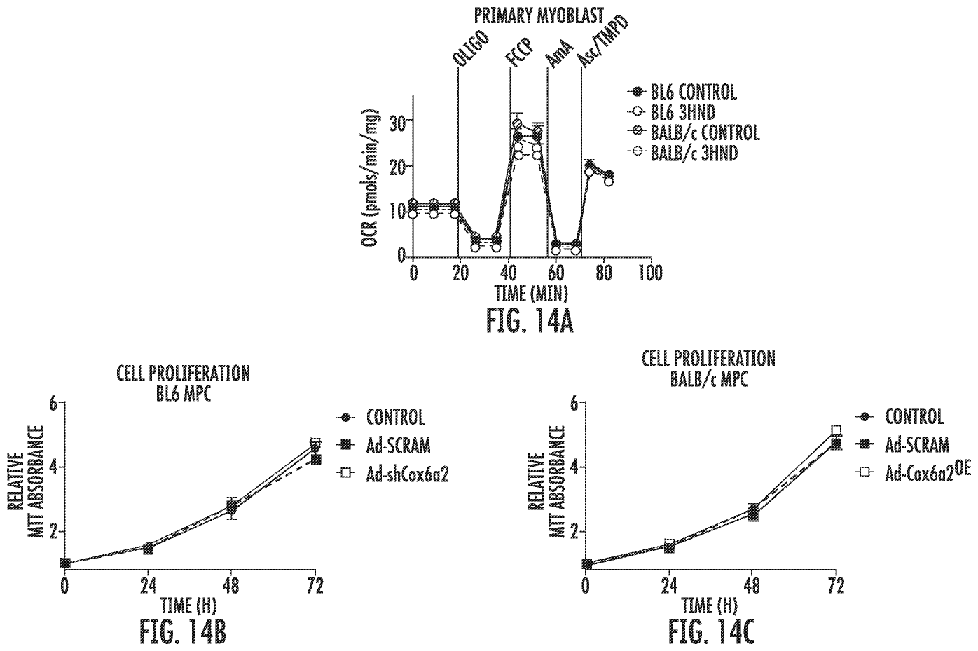

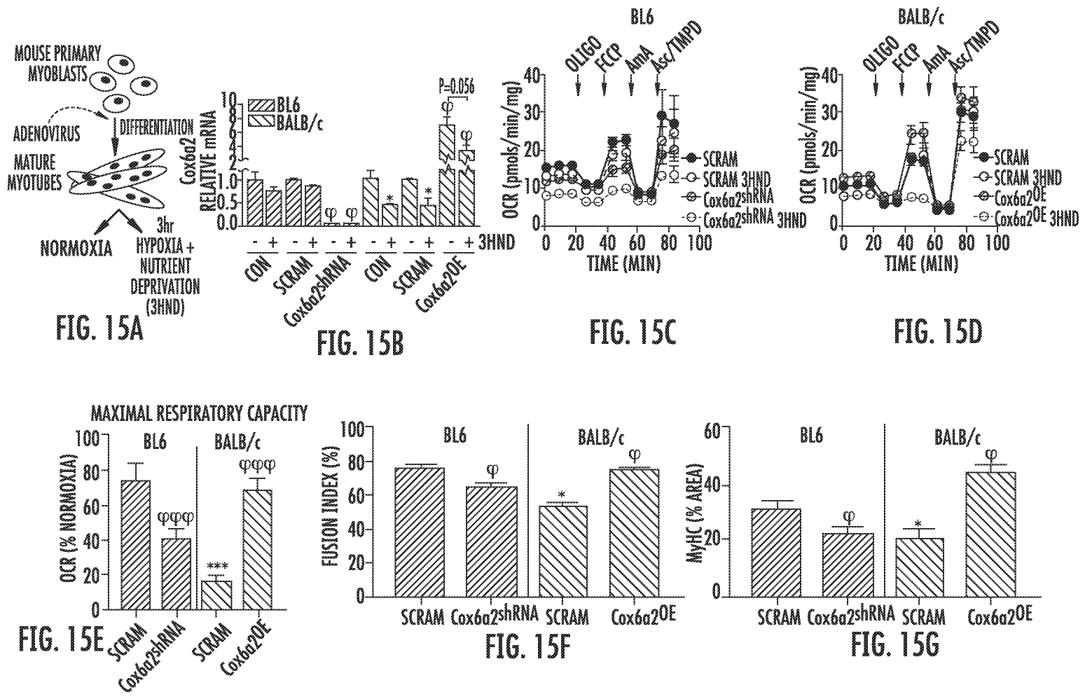

Cox6a2 is not believed to be expressed in muscle myoblasts until fusion and myotube maturation. This was verified in primary myoblasts based on Western blotting for Cox6a2 protein expression changes during differentiation in BALB/c primary muscle cells. Oxygen consumption under normal growth conditions and HND was assessed in undifferentiated primary muscle cells (myoblasts) from BL6 and BALB/c mice. In vitro ischemia did not alter undifferentiated MPCs (myoblasts) mitochondrial function from either BL6 or BALB/c mice. See FIG. 14A. To validate a specific role for Cox6a2 in the mitochondriopathy that befalls BALB/c primary MPCs, Cox6a2 expression was manipulated using adenoviruses encoding a scrambled construct (control), Cox6a2^{shRNA} (knockdown in BL6 cells), and Cox6a2^{OE} (overexpression in BALB/c cells) (see FIGS. 15A and 15B) and subjected to an in vitro ischemia model. More particularly, primary muscle cells were isolated from the hindlimb of BL6 and BALB/c mice and Cox6a2 expression was manipulated via adenovirus treatment during myoblast differentiation followed by either normoxia control or 3 hours hypoxia and nutrient deprivation to mimic PAD in vitro. Quantitative reverse transcription polymerase chain reaction (qRT-PCR) was performed to determine Cox6a2 mRNA expression levels in myotubes and cellular respiration was measured using a Seahorse XF24 extracellular flux analyzer (Agilent Technologies, Santa Clara, Calif., United States of America). Knockdown of Cox6a2 in myotubes decreased BL6 myotube maximal respiratory capacity. See FIGS. 15C and 15E. Overexpression of Cox6a2 in BALB/c myotubes increased myotube maximal respiratory capacity. See FIGS. 15D and 15E.

Because improved muscle regeneration in vivo was observed when delivering AAV9-Cox6a2 and Cox6 displays an isoform switch from Cox6a1 to Cox6a2 during myogenesis, a series of experiments were performed to explore if manipulation of Cox6a2 expression altered myoblast differentiation in vitro. Alterations in Cox6a2 expression (knockdown or overexpression) did not alter myoblast cell proliferation in BL6 or BABL/cJ mice, respectively. See FIGS. 14B and 14C. The fusion of myoblasts into myotubes was decreased in BALB/c MPCs compared with BL6. See FIG. 15F. Cox6a2 is in part responsible for the improved MPC differentiation in BL6 cells, as adenoviral knockdown of Cox6a2 decreased cell fusion and myotube size (myosin heavy chain positive area) in BL6 cells. See FIGS. 15F and 15G. Overexpression of Cox6a2 was sufficient to enhance myoblast fusion in BALB/c MPCs. See FIGS. 15F and 15G. Collectively, these results support a role for Cox6a2 in ischemic mitochondrial function in muscle myotubes and myofibers and provide critical in vitro evidence to support the importance of Cox6a2 and mitochondrial function in myogenesis.

### Example 10

### Cox6a2 Expression is Decreased in Human CLI Patients and is Required for Mitochondrial Respiratory Function in Human Muscle Cells

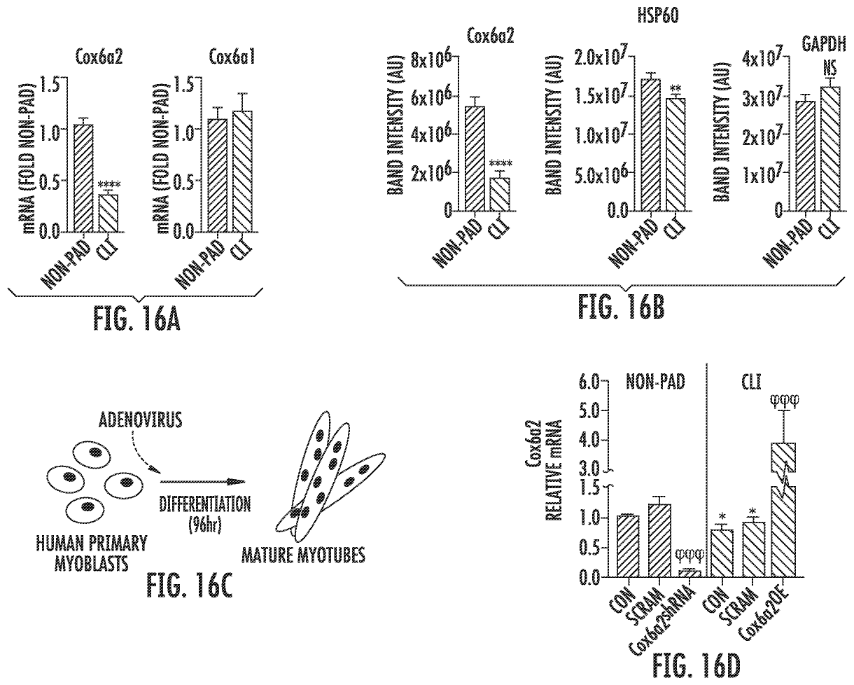

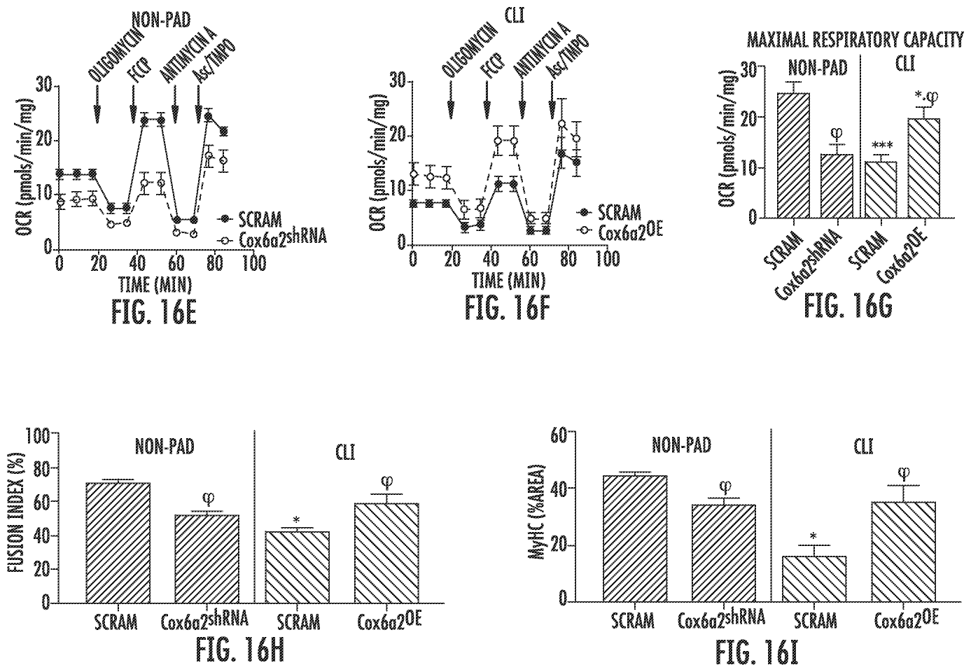

As described above, CLI patients suffer from a unique limb muscle cell mitochondriopathy. PAD muscle samples were collected from the lateral gastrocnemius muscle of patients with critical limb ischemia (CLI) undergoing limb amputation and age-matched controls without PAD (Non-PAD). Cox6a2 mRNA expression was significantly decreased in CLI muscle samples, whereas Cox6a1 (ubiquitous isoform expressed in many cell types) was unchanged. See FIG. 16A. Cox6a2 protein was also significantly reduced in CLI limb muscle (˜66% lower than Non-PAD. See FIG. 16B. In addition, the reduction in Cox6a2 protein was substantially larger than the observed alterations in mitochondrial content (indicated by mitochondrial matrix protein HSP60). See FIG. 16B.

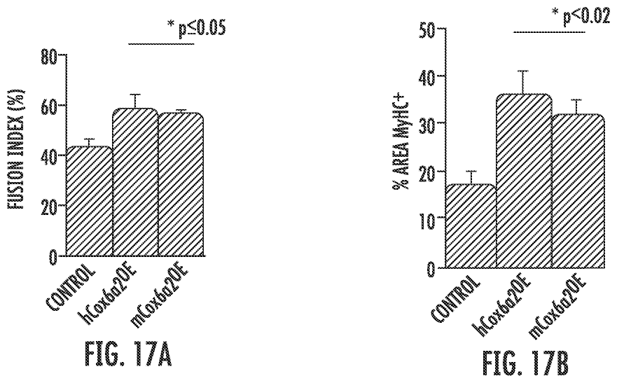

Using fresh muscle biopsy samples from non-PAD and CLI patients, primary human muscle cells were isolated and cultured from each group. See FIG. 16C. Adenovirus was utilized to manipulate Cox6a2 expression in these cells, which was verified by qRT-PCR. See FIG. 16D. Knockdown of Cox6a2 expression significantly decreased cell respiration in myotubes from non-PAD controls. See FIGS. 6E and 16G. In contrast, overexpression of Cox6a2 significantly increased cellular respiration in myotubes from CLI patients. See FIGS. 16F and 16G. Primary muscle cells from CLI patients displayed significantly lower rates of oxygen consumption compared with those from non-PAD patients. See FIG. 6G; both groups treated with same scrambled control adenovirus. Knockdown of Cox6a2 decreased myoblast fusion and size in MPCs from non-PAD controls; whereas Cox6a2 overexpression increased myoblast fusion and size in cells from CLI patients. See FIGS. 16H and 16I. In CLI MPCs, a similar increase in myotube fusion and size was observed when human cells were transfected with adenovirus encoding a human Cox6a2 protein compared with the murine Cox6a2 protein. See FIGS. 17A and 17B. Taken together, these data establish the translational relevance of Cox6a2 to the CLI patient limb muscles and confirm an important biological role for Cox6a2 expression in human muscle mitochondrial respiration and MPC differentiation.

### Example 11

### Polg^{+/+} Mice are Protected from Ischemic Myopathy

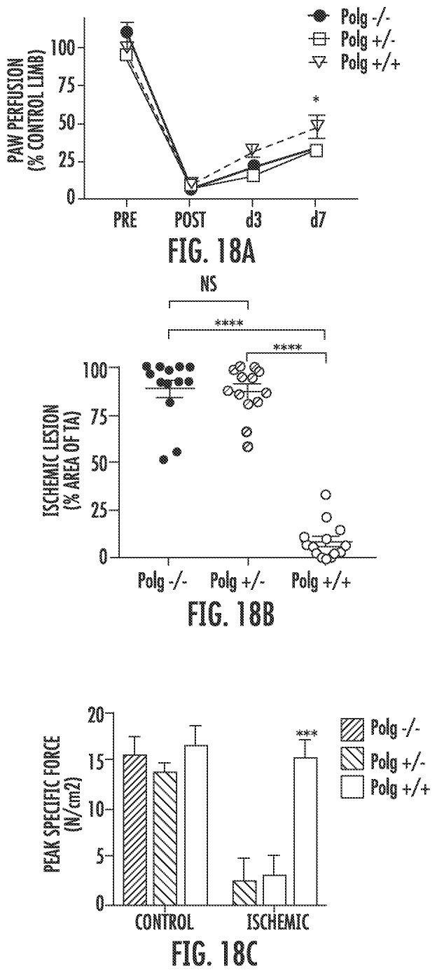

A transgenic mouse model was utilized to determine the effects of reduced mitochondrial health/function on ischemic injury. This transgenic mouse model harbors a mutant allele of mtDNA polymerase gamma (Polg) resulting in the gradual accumulation of mtDNA mutations that reduces mitochondrial function and shortens the lifespan of the mice to ˜13-15 months (obtained commercially from Jackson Laboratory). More particularly, wild type (WT, Polg^{-/-}), heterozygous (Polg^{+/-}), and homozygous (Polg^{+/+}) mice aged 12 months were subjected to HLI. In contrast to the hypothesis that reduced mitochondrial health/function that alters ischemic myopathy would exacerbate ischemic injury, it was observed that homozygous Polg^{+/+} mice (aged 12 months at the time of surgery) were completely protected from ischemic injury, demonstrated by improved ischemic limb blood flow, marked reductions in the ischemic lesion areas (total area of TA muscle with injury), and no impairment in muscle contractile function. See FIGS. 18A-18C. The degree of ischemia induced by HLI, confirmed by LDPI imaging, was not different between groups, confirming that Polg^{+/+} mice exhibit protection from ischemic injury.

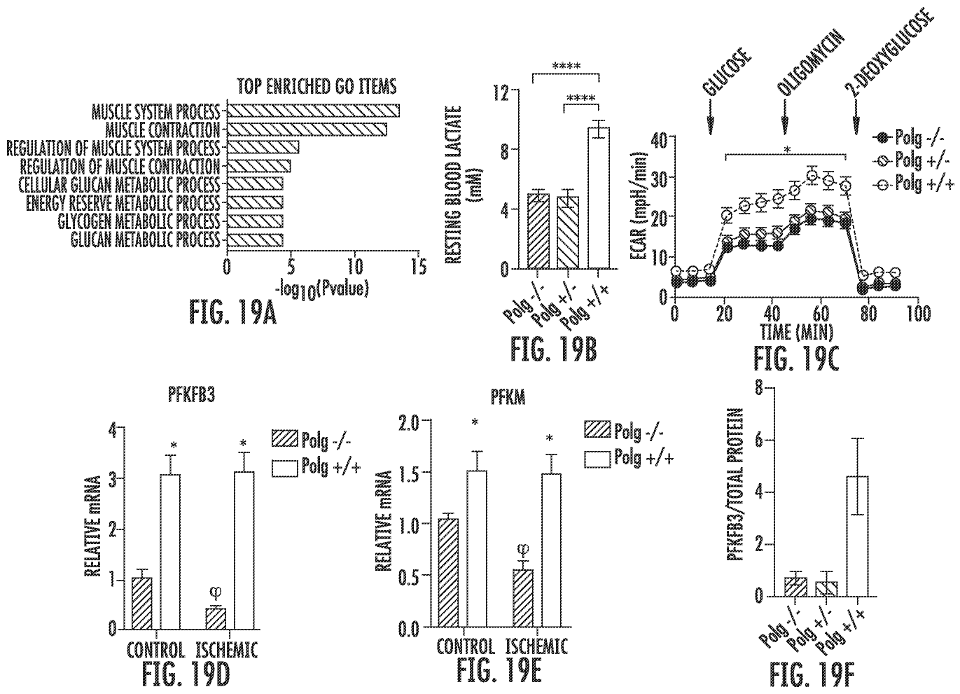

To identify potential mechanisms underlying this ischemic protection, whole transcriptome sequencing and differential gene expression analysis was performed in ischemic muscle (3 days post-surgery) from wildtype littermates and homozygous Polg^{+/+} mice. The Top Enriched Gene Ontology (GO) terms were related to either skeletal muscle structure/function or glucan (polysaccharides of glucose) metabolism. See FIG. 19A. Gene expression levels related to skeletal muscle structure/function were uniformly downregulated in the ischemic muscle from WT mice only (consistent with the degree of ischemic of injury). Polg^{+/+} mice displayed elevated gene expression of several genes indicating elevated non-oxidative/glycolytic metabolism in the control limb and preservation of expression following hindlimb ischemia (HLI). These findings highlighted two areas that have not been extensively explored in PAD: 1) processes involving skeletal muscle function are critical to limb pathology, and 2) glycolytic metabolism can play a role in the protection from ischemia. The RNA-seq data was followed up with metabolic phenotyping. A robust elevation in resting blood lactate levels (see FIG. 19B), enhanced glycolytic flux (ECAR, extracellular acidification rate—measured using a Seahorse XF analyzer (Agilent Technologies, Santa Clara, Calif., United States of America) in isolated primary skeletal muscle cells from Polg^{+/+} mice (see FIG. 19C), increased mRNA abundance of PFKFB3 and PFKM (see FIGS. 19D and 19E), and increased protein expression of PFKFB3 in ischemic muscle from Polg^{+/+} mice was observed. See FIG. 19F.

### Example 12

### AAV-Mediated Expression of PFKFB3 Increases Glycolytic Flux in Muscle and Endothelial Cells Resulting in Improved Hypoxia Tolerance and Angiogenesis In Vitro

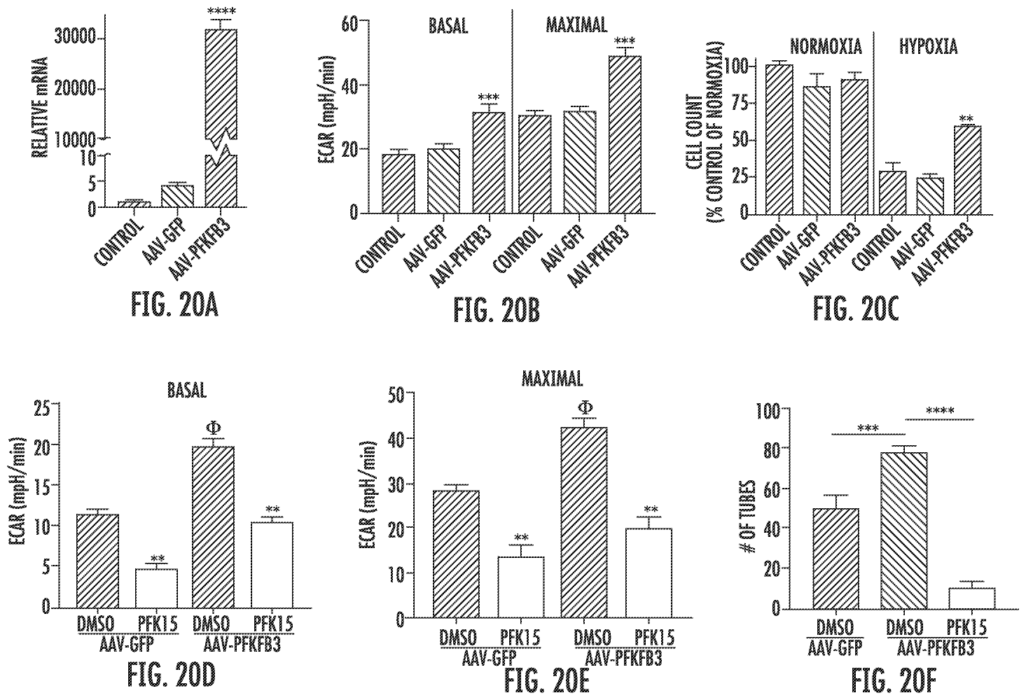

To begin examining the therapeutic potential of PFKFB3 in PAD, an AAV vector was generated to deliver PFKFB3 cloning the PFKFB3 coding sequence into an AAV2 ITR vector with expression driven by the ubiquitous cytomegalovirus promoter (CMV) promoter. AAV-PFKFB3 or control virus (AAV-DJ-GFP) was delivered to muscle (primary mouse myoblast) and endothelial (HUVEC) cells at a multiplicity of infection (MOI) of 10,000. Virus expression was validated by both mRNA and protein levels. See FIG. 20A. AAV delivery of PFKFB3 increased basal and maximal glycolytic flux rates in both muscle and endothelial cells. See FIGS. 20B, 20D, and 20E. The role of PFKFB3 as the primary mediator of the increased glycolytic flux in HUVECs appears to be confirmed by results of cells treated with the inhibitor (1-(4-pyridinyl)-3-(2-quinolinyl)-2-propen-1-one (PFK15). Muscle cells treated with AAV-DJ-PFKFB3 displayed greater hypoxia cell survival/viability (see FIG. 20C) compared with AAV-DJ-GFP treated cells. Moreover, endothelial cells treated with AAV-DJ-PFKFB3 displayed enhanced tube formation when plated on Matrigel (an in vitro model of angiogenesis) which was abolished when PFKFB3 was inhibited with PFK15. See FIG. 20F. Together, these in vitro findings highlight the therapeutic potential of PFKFB3 in both the muscle and endothelial cells, both of which are critical tissues in PAD pathology.

### Example 13

### AAV-Mediated Expression of PFKFB3 Decreases Ischemic Muscle Necrosis in BALB/cJ Mice

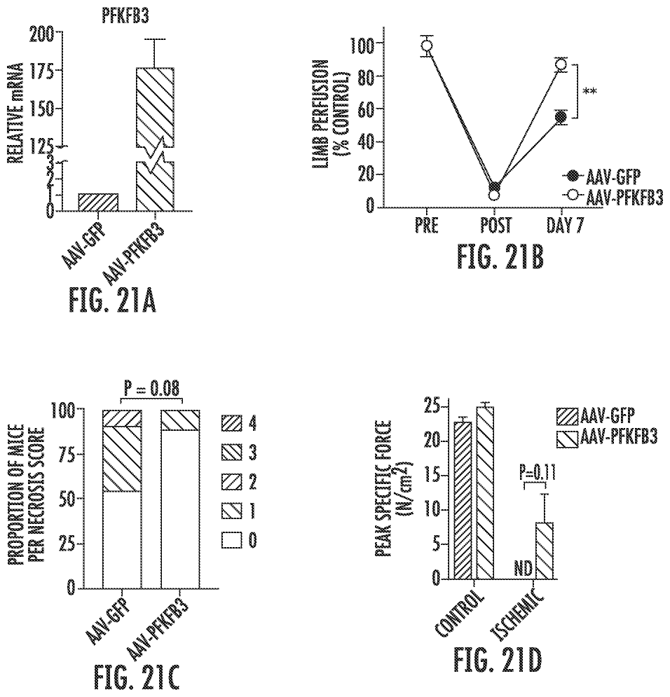

To further explore the therapeutic impact of PFKFB3 in PAD, a pre-clinical mouse model of hindlimb ischemia was used. AAV-GFP and AAV-PFKFB3 viruses (driven by the CMV promoter) were delivered to BALBc/J mice, which are known to respond poorly to limb ischemia. AAV expression was validated by IF microscopy for GFP and mRNA and protein level of PFKFB3 to confirm efficacy of the delivery by AAV injection. See FIG. 21A. Mice treated with AAV-PFKFB3 exhibited improved limb perfusion recovery measured by LDPI (see FIG. 21B) and lower degrees of tissue loss/necrosis. See FIG. 21C. Histological analysis of the skeletal muscle tissue indicated less ischemic muscle injury (larger myofiber, more myofibers with centralized nuclei, and less non-myofiber area), which contributed to a partial recovery in muscle force production. See FIG. 21D.

### Example 14

### PFKFB3 Expression is Decreased in CLI Patient Limbs

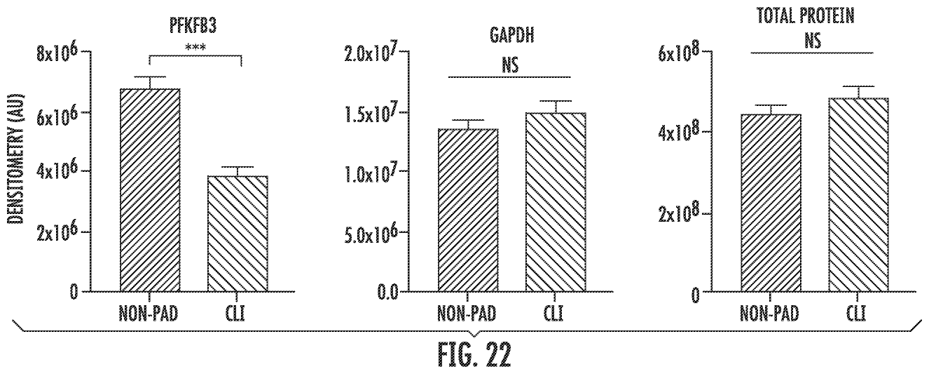

To further examine the clinical relevance of PFKFB3 as a therapeutic target, expression of PFKFB3 in PAD patients was examined. Limb muscle biopsy samples were obtained from aged-matched Non-PAD controls and severe PAD patients with critical limb ischemia (CLI). Levels of PFKFB3 expression were determined by Western blotting. CLI patients have decreased PFKFB3 expression in limb muscle biopsy samples (see FIG. 22), suggesting the association of PFKFB3 expression with development of CLI.

### Example 15

### Small Molecule Agents that Increase Glycolytic Metabolism

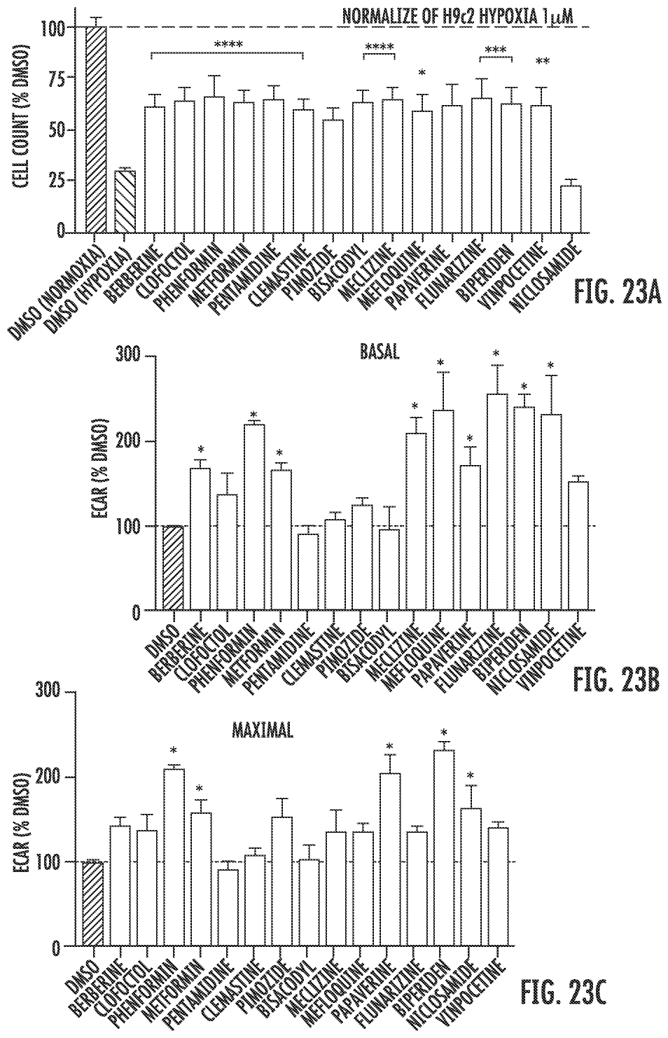

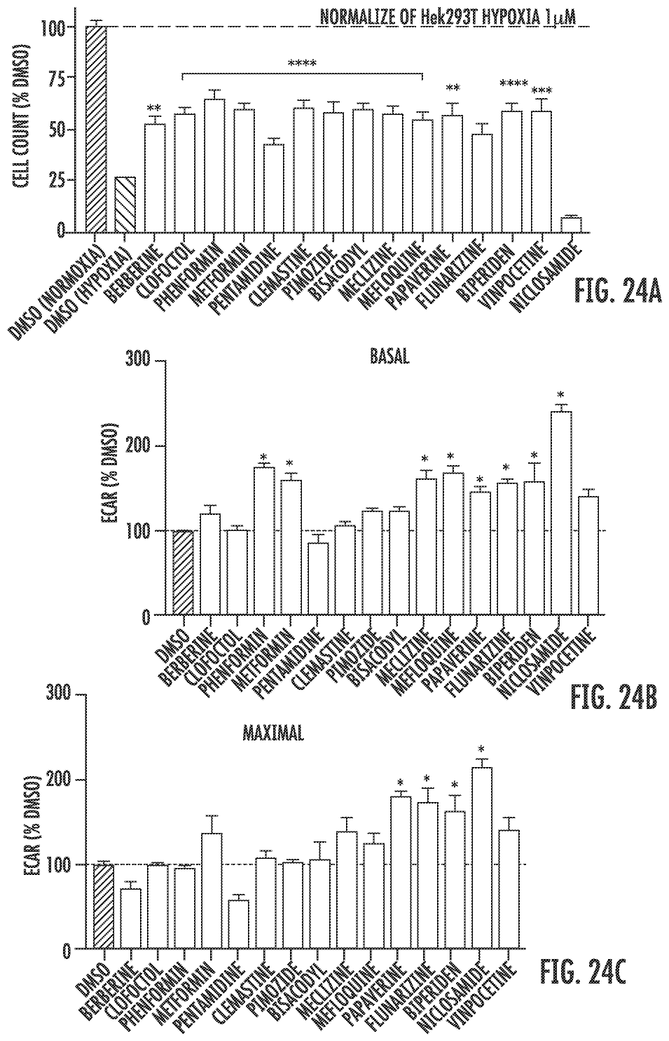

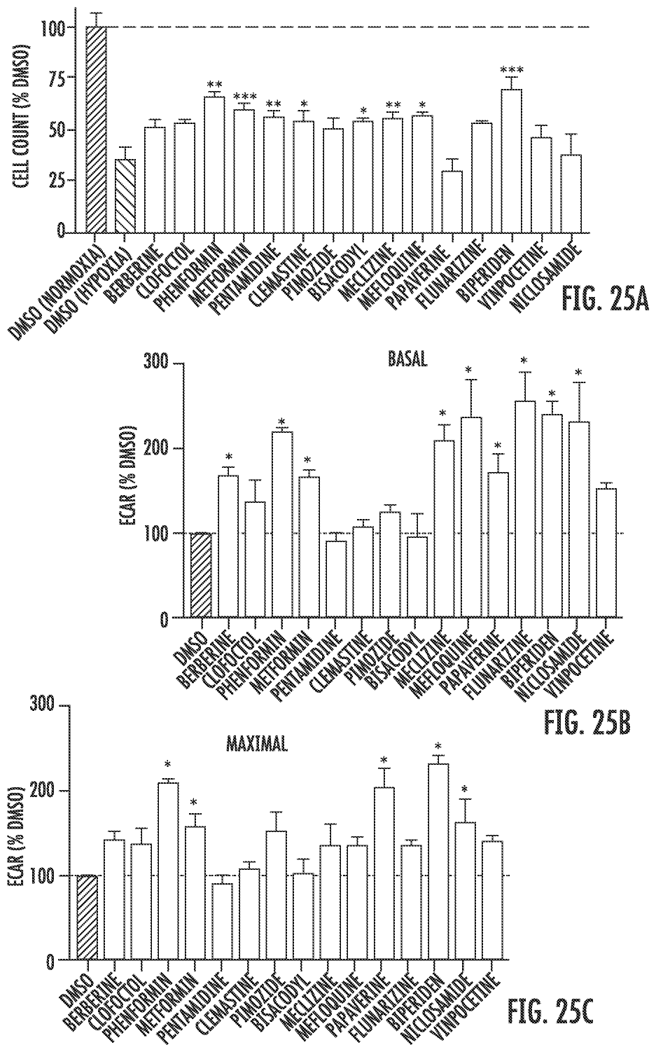

Many Food and Drug Administration (FDA) approved drugs and other chemical compounds have off-target effects on cellular metabolism, including the ability to enhance glycolytic metabolism. Cardiomyoblast (H9c2; obtained from ATCC) cells were treated with a number of compounds (berberine, clofoctol, phenformin, metformin, pentamidine, clemastine, pimoxide, bisacodyl, meclizine, mefloquine, papaverine, flunarizine, biperiden, vinpocetine, and niclosamide) at a final concentration of 1 μM prior to experimental hypoxia for 72 hours. Many of the compounds improved cell grown or survival, indicated by the number of cells counted following nuclear staining by DAPI. See FIG. 23A. Many of the compounds also improved glycolytic flux. Effects of the compounds on basal and maximal glycolytic flux are shown in FIGS. 23B and 23C. Human kidney fibroblast cells were also treated with the compound prior to experimental hypoxia. Again, most of the compounds improved cell growth or survival as indicated by the number of cells counted following nuclear staining with DAPI. See FIG. 24A. Effects of the compounds on basal and maximal glycolytic flux are shown in FIGS. 24B and 24C. In addition, primary mouse cortical neurons were treated with the compounds prior to experimental ischemia. Many of the compounds improved cell survival and glycolytic flux in these cells, as well. See FIGS. 25A-25C.

It will be understood that various details of the presently disclosed subject matter may be changed without departing from the scope of the presently disclosed subject matter. Furthermore, the foregoing description is for the purpose of illustration only, and not for the purpose of limitation.

1. A method of treating ischemia in a subject, the method comprising:
administering to the subject a composition comprising an agent adapted to modulate oxidative and/or glycolytic metabolism in ischemic cells in the subject; and
treating at least one symptom associated with the ischemia in the subject.

2. The method of claim 1, wherein treating at least one symptom associated with the ischemia comprises treating at least one symptom associated with ischemic injury, and wherein the symptom associated with ischemic injury comprises tissue necrosis, myopathy, fibrosis or vascular deficiency.

3. The method of claim 1, wherein the ischemia and/or ischemic injury is caused by one or more of stroke, myocardial infarction, ischemic limb pathology, peripheral arterial disease, including peripheral arterial disease compromising intermittent claudication, critical limb ischemia, trauma, diabetes mellitus, and acute limb ischemia.

4. The method of claim 1, wherein the agent is adapted to modulate oxidative metabolism in mitochondria in ischemic cells in the subject.

5. The method of claim 1, wherein the agent is adapted to provide Cox6a2 to the ischemic cells in the subject.

6. The method of claim 5, comprising administering a polynucleotide encoding a Cox6a2 polypeptide to the subject.

7. The method of claim 6, wherein the polynucleotide encoding the Cox6a2 polypeptide is operably coupled to a targeting vector capable of causing the expression of the Cox6a2 polypeptide in at least one of a muscle cell, fibroblast, stem cell, pericyte, and endothelial cell.

8. The method of claim 1, wherein the agent is adapted to modulate glycolytic metabolism in ischemic cells in the subject.

9. The method of claim 1, wherein the agent is adapted to provide PFKFB3 to the ischemic cells in the subject.

10. The method of claim 9, comprising administering a polynucleotide encoding a PFKFB3 polypeptide to the subject.

11. The method of claim 10, wherein the polynucleotide encoding the PFKFB3 polypeptide is operably coupled to a targeting vector capable of causing the expression of the PFKFB3 polypeptide in at least one of a muscle cell, fibroblast, stem cell, pericyte, and endothelial cell.

12. The method of claim 1, wherein the agent comprises one or more small molecule pharmacological agents.

13. The method of claim 12, wherein the one or more small molecule pharmacological agents are selected from the group consisting of metformin, phenformin, biperiden hydrochloride, clemastine, meclizine, berberine chloride, vinpocetine, pimozide and mefloquine.

14. The method of claim 1, wherein the composition comprises a liposome, a nanoparticle, plasmid DNA, recombinant adenovirus, recombinant adeno-associated virus, recombinant lentivirus and combinations thereof.

15. (canceled)

16. The method of claim 1, wherein administering the composition to the subject increases one or more of muscle fiber cross-sectional area, capillary density, muscle function, muscle regeneration, stem cell activity, vascular density, and vascular luminal diameter.

17. The method of claim 1, wherein administering the composition to the subject causes an increase in myotube diameter, a change in myotube phenotype, a change in contractile function, an increase in stem cell or satellite cell activity/myogenesis, an increase in mitochondrial number or respiratory function, an increase in autophagic flux, decreased DNA fragmentation or combinations thereof.

18. The method of claim 1, wherein administering the composition to the subject causes one or more of increased expression of vascular endothelial growth factor (VEGF), neuropilin (Nrp-1), vascular endothelial growth factor receptor 1 (Flt), vascular endothelial growth factor receptor 2 (Flk), myogenin, myoD, Tmem8c (myomaker) and muscle RING-finger protein 1 (MuRF-1), PGC1-alpha, opa1, Drp1, Mitofusion (Mfn) 1 or 2 and decreased in expression of myostatin.

19. The method of claim 1, wherein the method further comprises, prior to administering to the subject the composition comprising an agent adapted to modulate oxidative and/or glycolytic metabolism in ischemic cells:
(i) obtaining a sample from the subject, wherein said sample comprises myofibers from muscle; and
(ii) measuring mitochondrial function in the sample from said subject.

20. The method of claim 19, wherein measuring mitochondrial function in the sample comprises determining one or more of a Complex IV oxygen consumption rate below 2,000 picomoles per second per milligram myofiber, a Complex II₃ oxygen consumption rate of below 1,000 picomoles per second per milligram myofiber, and/or a Complex I+II₃ oxygen consumption rate of below 1,000 picomoles per second per milligram microfiber.

21. (canceled)

22. (canceled)

23. A method of classifying a subject having peripheral arterial disease (PAD) as unlikely to respond to an endovascular therapeutic intervention, a revascularization therapeutic intervention and/or a therapeutic intervention comprising physical activity, the method comprising:
providing a sample from a subject having PAD, wherein the sample comprises myofibers from skeletal muscle;
measuring mitochondrial function in the sample; and
classifying the subject as being unlikely to respond to an endovascular therapeutic intervention, a revascularization therapeutic intervention and/or a therapeutic intervention comprising physical activity based on mitochondrial function.

24. (canceled)


SEQUENCE LISTING (8 sequences)

SEQ ID NO: 1 - DNA, 294 bp, Mus musculus
atggctctgcctctaaaggtcctgagccggagcatggccagcgcagccaaaggagaccatggaggggcaggagccaacacctggcgcctcctgacctttgtgctggctcttcccggcgtagccctctgctcccttaactgctggatgcacgctggccaccacgagcgcccagagttcatcccgtatcaccacctccgcatccgaaccaagcccttcgcctggggggacggcaaccacacgcttttccacaatccccacgtcaatcctttgcccaccggttatgagcacccttga

SEQ ID NO: 2 - protein, 97 aa, Mus musculus
MALPLKVLSRSMASAAKGDHGGAGANTWRLLTFVLALPGVALCSLNCWMHAGHHERPEFIPYHHLRIRTKPFAWGDGNHTLFHNPHVNPLPTGYEHP

SEQ ID NO: 3 - DNA, 294 bp, Homo sapiens
atggctttgcctctgaggcccctgacccggggcttggccagcgctgccaaaggaggccacggaggagcaggagctcgtacctggcgtctgctgaccttcgtgctggcgctgcccagcgtggccctctgcaccttcaactcctatctccactcgggccaccgcccgcgccccgagttccgtccctaccaacacctccgcatccgcaccaagccctacccctggggggacggcaaccacactctgttccacaatagccacgtgaaccctctgcccacgggctacgaacacccctga

SEQ ID NO: 4 - protein, 97 aa, Homo sapiens
MALPLRPLTRGLASAAKGGHGGAGARTWRLLTFVLALPSVALCTFNSYLHSGHRPRPEFRPYQHLRIRTKPYPWGDGNHTLFHNSHVNPLPTGYEHP

SEQ ID NO: 5 - DNA, 1581 bp, Mus musculus
atgccgttggaactgacccagagccgggtacagaagatctgggtgcccgtcgatcaccgaccctcgttgcccagatcctgtgggccgaagctgaccaactccccaaccgtgattgtgatggtggggctcccagcccggggtaagacttacatctccaagaagctgactcgctacctcaactggataggtgttccaacgaaagtgttcaatgtgggagagtatcggcgtgaggctgtgaagcagtacagctcttacaacttcttccgccctgacaatgaggaagccatgagagtccgaaaacagtgtgccttagctgccctgagagatgtcaaaagctacctgacaaaggaaggtggacagattgcagtttttgatgccaccaatacaactagagaaagaaggcacatgatccttaattttgccaaagaaaatgacttcaaggcatttttcatcgagtcggtctgtgacgaccctactgttgtggcctccaacatcatggaagttaaaatctccagcccggattacaaagactgcaactcagcagaagccatggacgacttcatgaagagaatcaattgctatgaagccagctaccagcctcttgaccctgataaatgtgacagggacttgtcactcatcaaagtgattgatgttggccggaggttcttggtgaacagagtgcaggaccacatccagagccgaattgtatactacctgatgaacatccacgtgcagccccgtaccatctacctgtgtcggcatggcgagaatgagtacaacttgcagggcaagattgggggcgactcaggcttgtccagcagaggcaagaagttcgccaatgccctgagcaagttcgtagaagaacagaatctgaaggacctccgtgtgtggaccagccagctgaaaagtaccattcagaccgcggaggccctgaggctcccctacgagcagtggaaggcactcaacgagatcgatgctggtgtgtgtgaggaactaacctatgaggagatcagagatacctaccctgaggagtacgcactgcgtgaacaggacaagtactactatcgctatcccacgggagagtcctaccaggatcttgtccaacgcctggagcctgtgatcatggaattagagcgccaagagaatgtattggtcatctgtcaccaggctgttctacgctgcctactagcctacttcctcgacaagagtgcagaggagatgccatacctgaaatgtccgctccacactgtcctgaaactgacacctgtggcatatgggtgccgggtggagtccatttacttgaatgtagaatcggtgagcacacaccgggagaggtcagaggatgcaaagaagggacctaacccgctcatgagacgcaatagtgtcaccccactagccagtcccgagcccaccaaaaagcctcgcatcaacagctttgaggagcgtgtggcttccacttctgctgccctgccaagctgcctgcccccggaagtgcccacgcagctgcccggacaacctttgctagggaaagcctgtctacgaagtgtctgtcacattttttcaaagttttctccttactaa

SEQ ID NO: 6 - protein, 526 aa, Mus musculus
MPLELTQSRVQKIWVPVDHRPSLPRSCGPKLTNSPTVIVMVGLPARGKTYISKKLTRYLNWIGVPTKVFNVGEYRREAVKQYSSYNFFRPDNEEAMRVRKQCALAALRDVKSYLTKEGGQIAVFDATNTTRERRHMILNFAKENDFKAFFIESVCDDPTVVASNIMEVKISSPDYKDCNSAEAMDDFMKRINCYEASYQPLDPDKCDRDLSLIKVIDVGRRFLVNRVQDHIQSRIVYYLMNIHVQPRTIYLCRHGENEYNLQGKIGGDSGLSSRGKKFANALSKFVEEQNLKDLRVWTSQLKSTIQTAEALRLPYEQWKALNEIDAGVCEELTYEEIRDTYPEEYALREQDKYYYRYPTGESYQDLVQRLEPVIMELERQENVLVICHQAVLRCLLAYFLDKSAEEMPYLKCPLHTVLKLTPVAYGCRVESIYLNVESVSTHRERSEDAKKGPNPLMRRNSVTPLASPEPTKKPRINSFEERVASTSAALPSCLPPEVPTQLPGQPLLGKACLRSVCHIFSKFSPY

SEQ ID NO: 7 - DNA, 1563 bp, Homo sapiens
atgccgttggaactgacgcagagccgagtgcagaagatctgggtgcccgtggaccacaggccctcgttgcccagatcctgtgggccaaagctgaccaactcccccaccgtcatcgtcatggtgggcctccccgcccggggcaagacctacatctccaagaagctgactcgctacctcaactggattggcgtccccacaaaagtgttcaacgtcggggagtatcgccgggaggctgtgaagcagtacagctcctacaacttcttccgccccgacaatgaggaagccatgaaagtccggaagcaatgtgccttagctgccttgagagatgtcaaaagctacctggcgaaagaagggggacaaattgcggttttcgatgccaccaatactactagagagaggagacacatgatccttcattttgccaaagaaaatgactttaaggcgtttttcatcgagtcggtgtgcgacgaccctacagttgtggcctccaatatcatggaagttaaaatctccagcccggattacaaagactgcaactcggcagaagccatggacgacttcatgaagaggatcagttgctatgaagccagctaccagcccctcgaccccgacaaatgcgacagggacttgtcgctgatcaaggtgattgacgtgggccggaggttcctggtgaaccgggtgcaggaccacatccagagccgcatcgtgtactacctgatgaacatccacgtgcagccgcgtaccatctacctgtgccggcacggcgagaacgagcacaacctccagggccgcatcgggggcgactcaggcctgtccagccggggcaagaagtttgccagtgctctgagcaagttcgtggaggagcagaacctgaaggacctgcgcgtgtggaccagccagctgaagagcaccatccagacggccgaggcgctgcggctgccctacgagcagtggaaggcgctcaatgagatcgacgcgggcgtctgtgaggagctgacctacgaggagatcagggacacctaccctgaggagtatgcgctgcgggagcaggacaagtactattaccgctaccccaccggggagtcctaccaggacctggtccagcgcttggagccagtgatcatggagctggagcggcaggagaatgtgctggtcatctgccaccaggccgtcctgcgctgcctgcttgcctacttcctggataagagtgcagaggagatgccctacctgaaatgccctcttcacaccgtcctgaaactgacgcctgtcgcttatggctgccgtgtggaatccatctacctgaacgtggagtccgtctgcacacaccgggagaggtcagaggatgcaaagaagggacctaacccgctcatgagacgcaatagtgtcaccccgctagccagccccgaacccaccaaaaagcctcgcatcaacagctttgaggagcatgtggcctccacctcggccgccctgcccagctgcctgcccccggaggtgcccacgcagctgcctggacaaaacatgaaaggctcccggagcagcgctgactcctccaggaaacactga

SEQ ID NO: 8 - protein, 520 aa, Homo sapiens
MPLELTQSRVQKIWVPVDHRPSLPRSCGPKLTNSPTVIVMVGLPARGKTYISKKLTRYLNWIGVPTKVFNVGEYRREAVKQYSSYNFFRPDNEEAMKVRKQCALAALRDVKSYLAKEGGQIAVFDATNTTRERRHMILHFAKENDFKAFFIESVCDDPTVVASNIMEVKISSPDYKDCNSAEAMDDFMKRISCYEASYQPLDPDKCDRDLSLIKVIDVGRRFLVNRVQDHIQSRIVYYLMNIHVQPRTIYLCRHGENEHNLQGRIGGDSGLSSRGKKFASALSKFVEEQNLKDLRVWTSQLKSTIQTAEALRLPYEQWKALNEIDAGVCEELTYEEIRDTYPEEYALREQDKYYYRYPTGESYQDLVQRLEPVIMELERQENVLVICHQAVLRCLLAYFLDKSAEEMPYLKCPLHTVLKLTPVAYGCRVESIYLNVESVCTHRERSEDAKKGPNPLMRRNSVTPLASPEPTKKPRINSFEEHVASTSAALPSCLPPEVPTQLPGQNMKGSRSSADSSRKH